(19) United States
(12) Patent Application Publication
(10) Pub. No.: US 2026/0308238 A1
(43) Pub. Date: Oct. 8, 2026

(54) PEDIATRIC DOSAGE AND ADMINISTRATION OF ANTI-C5 ANTIBODIES FOR TREATMENT OF COMPLEMENT DISORDERS

(71) Applicant: Alexion Pharmaceuticals, Inc., Boston, MA (US)

(72) Inventors: Stephan ORTIZ, Boston, MA (US); Jonathan MONTELEONE, Boston, MA (US)

(21) Appl. No.: 19/480,075

(22) PCT Filed: Apr. 30, 2024
(86) PCT No.: PCT/US2024/027004

§ 371 Date: Oct. 30, 2025

Related U.S. Application Data
(60) Provisional application No. 63/499,599, filed on May 2, 2023.

(51) Int. Cl. C07K 16/18; A61K 39/00; A61K 47/02; A61K 47/18; A61K 47/26; A61M 5/142; A61M 5/20; A61M 5/315

(52) U.S. Cl. CPC C07K 16/18; A61K 47/02; A61K 47/183; A61K 47/26; A61M 5/14244; A61M 5/20; A61M 5/315; A61K 2039/54; A61K 2039/545; A61K 2039/55; A61M 2005/14268; C07K 2317/565

(57) ABSTRACT

Provided are devices, systems, and methods for clinical treatment of complement disorders (e.g., paroxysmal nocturnal hemoglobinuria and atypical hemolytic uremic syndrome) in a pediatric human patient using subcutaneous delivery systems (e.g., on-body device and/or syringe) to administer an effective amount of a therapeutic anti-C5 antibody such as ravulizumab, or antigen-binding fragment thereof.

### CROSS-REFERENCE TO RELATED APPLICATIONS

This application claims priority to, and the benefit of, U.S. Provisional Application No. 63/499,599, filed May 2, 2023, which is incorporated by reference herein in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Feb. 17, 2023, is named 51196-034001_Sequence_Listing_2_17_23 and is 49,397 bytes in size.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16th Edition.

While a properly functioning complement system provides a robust defense against infecting microbes, inappropriate regulation or activation of the complement pathways has been implicated in the pathogenesis of a variety of disorders, including paroxysmal nocturnal hemoglobinuria (PNH) and atypical hemolytic uremic syndrome (aHUS). PNH and aHUS are both ultra-rare disorders driven by chronic uncontrolled complement activation The resulting inflammation and cellular damage lead to the devastating clinical manifestations of these diseases.

PNH is a condition in which uncontrolled complement activity leads to systemic complications, principally through intravascular hemolysis and platelet activation (see Sociè G, et al., French Society of Haematology. Lancet. 1996; 348(9027):573-577 and Brodsky, R., Blood. 2014; 124(18).2804-2811). Persistent intravascular hemolysis may be triggered by various stressors, such as infection or physical exertion, which leads to smooth muscle contraction (free hemoglobin), chronic anemia, and an increased risk of severe thromboembolism Thromboembolism, as the most common cause of mortality in patents with PNH, may lead to pulmonary hypertension and end-organ damage of vital organs, such as the liver, kidneys, brain, and intestines (Hillmen, P., et al, Am. J. Hematol. 2010; 85(8):553-559). Due to these adverse pathologic processes, patients with PNH have a decreased quality of life (QoL), which may include debilitating fatigue, chronic pain, poor physical function, shortness of breath, abdominal pain, erectile dysfunction, a need for anticoagulation, blood transfusions and in some instances, a need for dialysis (Weitz, I C., et al., Thromb Res. 2012; 130(3):361-368).

Hemolytic uremic syndrome (HUS) is characterized by thrombocytopenia, microangiopathic hemolytic anemia, and acute renal failure. HUS is classified as one of two types: diarrheal-associated (D+ HUS; also referred to as shiga toxin producing E. coli (STEC)-HUS or typical HUS) and non-diarrheal or atypical HUS (aHUS). D+ HUS is the most common form, accounting for greater than 90% of cases and is caused by a preceding illness with a shiga-like toxin-producing bacterium, e.g., E. coli O157:H7.

aHUS can be genetic, acquired, or idiopathic. Hereditable forms of aHUS can be associated with mutations in a number of human complement components including, e.g., complement factor H (CFH), membrane cofactor protein (MCP), complement factor I (CFI), C4b-binding protein (C4BP), complement factor B (CFB), and complement component 3 (C3). See, e.g., Caprioli et al. (2006) Blood 108:1267-1279. Certain mutations in the gene encoding CD55, though not yet implicated in aHUS, are associated with the severity of aHUS. See, e.g., Esparza-Gordillo et al. (2005) Hum Mol Genet 14:703-712.

aHUS is rare and has a mortality rate of up to 25%. Many patients with this disease will sustain permanent neurological or renal impairment, e.g., at least 50% of aHUS patients progress to end-stage renal failure (ESRF). See, e.g., Kavanagh et al. (2006) British Medical Bulletin 77 and 78:5-22. Until recently, treatment options for patients with aHUS were limited and often involved plasma infusion or plasma exchange. In some cases, aHUS patients undergo uni- or bilateral nephrectomy or renal transplantation (see Artz et al. (2003) Transplantation 76:821-826). However, recurrence of the disease in treated patients is common.

Patients with PNH or aHUS are at risk of substantial morbidity and mortality. Accordingly, it is an object of the present invention to provide improved methods for treating patients with PNH or aHUS.

### SUMMARY

Provided herein are, inter alia, devices, compositions, methods, and kits for treating PNH or aHUS in a pediatric human patient, including subcutaneously administering to the patient a therapeutic anti-C5 antibody, or antigen-binding fragment thereof, wherein the anti-C5 antibody, or antigen-binding fragment thereof, is administered (or is for administration) according to a particular clinical dosage regimen (i.e., at a particular dose amount and according to a specific dosing schedule).

Any suitable anti-C5 antibody, or antigen-binding fragment thereof, can be used in the methods described herein. An exemplary anti-C5 antibody is ravulizumab (also known as Ultomiris™, ALXN1210 and antibody BNJ441) including the heavy and light chains having the sequences shown in SEQ ID NOs: 14 and 11, respectively, or antigen binding fragments and variants thereof. In other embodiments, the antibody includes the heavy and light chain complementarity determining regions (CDRs) or variable regions (VRs) of ravulizumab. Accordingly, in one embodiment, the antibody includes the CDR1, CDR2, and CDR3 domains of the heavy chain variable (VH) region of ravulizumab having the sequence shown in SEQ ID NO: 12, and the CDR1, CDR2 and CDR3 domains of the light chain variable (VL) region of ravulizumab having the sequence shown in SEQ ID NO: 8. In another embodiment, the antibody includes CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively. In another embodiment, the antibody includes VH and VL regions having the amino acid sequences set forth in SEQ ID NO: 12 and SEQ ID NO: 8, respectively. In another embodiment, the antibody includes a heavy chain constant region as set forth in SEQ ID NO: 13.

In a first aspect, the invention features an on-body delivery system (OBDS) configured for administration of an effective amount of a therapeutic anti-C5 antibody, or antigen-binding fragment thereof, including complementarity determining region (CDR)1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat atypical hemolytic uremic syndrome (aHUS) or paroxysmal nocturnal hemoglobinuria (PNH) in a pediatric human patient in need thereof, wherein the patient is complement inhibitor treatment-naïve or eculizumab-experienced, wherein the therapeutic anti-C5 antibody, or antigen-binding fragment thereof, is administered subcutaneously once per week at a dose of:
(i) 245 mg to a patient weighing ≥20 to <40 kg; or
(ii) 490 mg to a patient weighing ≥40 kg.

In another aspect, the invention features an OBDS configured for treating aHUS or PNH in a pediatric human patient in need thereof, wherein the OBDS includes an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is complement inhibitor treatment-naïve or eculizumab-experienced, and wherein the OBDS comprises means for subcutaneous administration of:
(i) 245 mg anti-C5 antibody, or antigen-binding fragment thereof, to a patient weighing ≥20 to <40 kg; or
(ii) 490 mg anti-C5 antibody, or antigen-binding fragment thereof, to a patient weighing ≥40 kg.

In another aspect, the invention features an OBDS configured for administration of an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat aHUS or PNH in a pediatric human patient in need thereof, wherein the patient is ravulizumab-experienced, wherein the therapeutic anti-C5 antibody, or antigen-binding fragment thereof, is administered subcutaneously once per week at a dose of.
(i) 245 mg to a patient weighing ≥20 to <40 kg; or
(ii) 490 mg to a patient weighing ≥40 kg.

In another aspect, the invention features an OBDS configured for treating aHUS or PNH in a pediatric human patient in need thereof, wherein the OBDS includes an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ravulizumab-experienced, and wherein the OBDS comprises means for subcutaneous administration of:
(i) 245 mg anti-C5 antibody, or antigen-binding fragment thereof, to a patient weighing ≥20 to <40 kg, or
(ii) 490 mg anti-C5 antibody, or antigen-binding fragment thereof, to a patient weighing ≥40 kg.

In some embodiments of any of the foregoing aspects, the pediatric human patient is less than 21 years of age. In some embodiments, the pediatric human patient is between 2 and 18 years of age.

In some embodiments of any of the foregoing aspects, the OBDS includes a cartridge including the therapeutic anti-C5 antibody, or antigen-binding fragment thereof, and an on-body injector (OBI). In some embodiments, the OBDS is configured for administration of the therapeutic anti-C5 antibody, or antigen-binding fragment thereof, subcutaneously to a patient weighing ≥20 to <40 kg at a dose of 245 mg using one OBDS. In some embodiments, the 245 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% weight by volume (w/v) sucrose, 0.05% w/v polysorbate 80 (PS80), and water for injection, wherein the OBDS includes one OBI and one pre-filled cartridge containing 245 mg of the therapeutic anti-C5 antibody or antigen-binding fragment thereof.

In some embodiments of any of the foregoing aspects, the OBDS is configured for administration of the therapeutic anti-C5 antibody, or antigen-binding fragment thereof, subcutaneously to a patient weighing ≥40 kg at a dose of 490 mg in combination with a second OBDS. In some embodiments, the 490 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, wherein each OBDS includes one OBI and one pre-filled cartridge containing 245 mg of the therapeutic anti-C5 antibody or antigen-binding fragment thereof.

In some embodiments of any of the foregoing aspects, the OBDS is a single-use electro-mechanical medical device including: an injector including a 29-gauge needle and a cartridge including a piston and telescopic screw assembly. In some embodiments, the cartridge has a volume of 3.5 mL. In some embodiments, the cartridge has a volume of 10 mL. In some embodiments, the OBDS is configured to deliver the therapeutic anti-C5 antibody, or antigen-binding fragment thereof, via subcutaneous injection in about 10 minutes. In some embodiments, the cartridge includes the therapeutic anti-C5 antibody, or antigen-binding fragment thereof, and an opening sealed by a pierceable septum.

In some embodiments of any of the foregoing aspects, the OBDS includes an OBI including:
(i) an injector housing;
(ii) an injection needle translatable between a retracted position, wherein at least a tip of the injection needle is contained within the injector housing, and an injection position, wherein at least the tip of the injection needle protrudes from the injector housing;
(iii) an activation button assembly movably mounted to the injector housing and operatively connected to the injection needle, the activation button assembly being translatable from an unactuated position to an actuated position to drive the injection needle from the retracted position thereof to the injection position thereof;
(iv) a cartridge door movably mounted to the injector housing between an open position and a closed position, the cartridge door including:
   an open end,
   an interior channel having a cartridge mounted therein, the cartridge containing the therapeutic anti-C5 antibody, or antigen-binding fragment thereof, to be dispensed and having an opening at a front end of the cartridge sealed by a pierceable septum and a flange at a rear end thereof, and
   a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and
(v) a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum;
wherein the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum.

In some embodiments of any of the foregoing aspects, the OBDS is configured for self-administration of the therapeutic anti-C5 antibody, or antigen-binding fragment thereof, subcutaneously to the patient. In some embodiments, the OBDS is configured for subcutaneous administration of the therapeutic anti-C5 antibody, or antigen-binding fragment thereof, to a site selected from the patient's arm, abdomen, and thigh.

In another aspect, the invention features a needle syringe configured for administration of an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat aHUS or PNH in a pediatric human patient in need thereof, wherein the patient is ≥10 to <20 kg of body weight and is complement inhibitor treatment-naïve, eculizumab-experienced, or ravulizumab-experienced, wherein the therapeutic anti-C5 antibody, or antigen-binding fragment thereof, is administered subcutaneously once per week at a dose of 150 mg. In some embodiments, the antibody formulation (e.g., 100 mg/ml ravulizumab) administered by syringe is not diluted prior to administration.

In yet another aspect, the invention features a needle syringe configured for treating aHUS or PNH in a pediatric human patient in need thereof, wherein the needle syringe is configured for subcutaneous administration and includes 150 mg of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is 10 to <20 kg of body weight and is complement inhibitor treatment-naïve, eculizumab-experienced, or ravulizumab-experienced, and wherein the needle syringe subcutaneously delivers 150 mg anti-C5 antibody, or antigen-binding fragment thereof.

In some embodiments of any of the foregoing aspects, the 150 mg dose of the therapeutic anti-C5 antibody, or antigen-binding fragment thereof is formulated at pH 7.4 and a concentration of 100 mg/mL in sodium phosphate, sucrose, L-arginine, PS80, and water for injection. In some embodiments, the needle syringe is configured for administration of the therapeutic anti-C5 antibody, or antigen-binding fragment thereof, to a site selected from the patient's arm, abdomen, and thigh.

In some embodiments of any of the foregoing aspects, the therapeutic anti-C5 antibody, or antigen-binding fragment thereof, includes a variant human Fc constant region that binds to human neonatal Fc receptor (FcRn), wherein the variant human Fc CH3 constant region includes Met-429-Leu and Asn-435-Ser substitutions at residues corresponding to methionine 428 and asparagine 434 of a native human IgG Fc constant region, each in EU numbering. In some embodiments, the therapeutic anti-C5 antibody, or antigen-binding fragment thereof, includes a heavy chain variable region including the amino acid sequence of SEQ ID NO: 12 and a light chain variable region including the amino acid sequence of SEQ ID NO: 8. In some embodiments, the therapeutic anti-C5 antibody, or antigen-binding fragment thereof, further includes a heavy chain constant region including in the amino acid sequence of SEQ ID NO: 13. In some embodiments, the therapeutic anti-C5 antibody, or antigen-binding fragment thereof, includes a heavy chain polypeptide including the amino acid sequence of SEQ ID NO: 14 and a light chain polypeptide including the amino acid sequence of SEQ ID NO: 11. In some embodiments, the heavy chain variable region includes an N-terminal pyroglutamate residue.

In another aspect, the invention features a kit comprising the OBDS of any of the foregoing aspects or the needle syringe of any of the foregoing aspects and instructions for using the OBDS or the needle syringe for treatment of PNH or aHUS.

In another aspect, the invention features a method of treating a pediatric human patient with PNH or aHUS, wherein the patient is complement inhibitor treatment-naïve or eculizumab-experienced, the method including administering to the patient during an administration cycle an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
(a) intravenously once on Day 1 of the administration cycle at a dose of:
   (i) 600 mg to a patient weighing ≥10 to <20 kg;
   (ii) 900 mg to a patient weighing ≥20 to <30 kg;
   (iii) 1200 mg to a patient weighing ≥30 to <40 kg;
   (iv) 2400 mg to a patient weighing ≥40 to <60 kg; or
   (v) 2700 mg to a patient weighing ≥60 kg, and
(b) subcutaneously on Day 15 of the administration cycle and every week thereafter at a dose of:
   (i) 150 mg to a patient weighing ≥10 to <20 kg;
   (ii) 245 mg to a patient weighing ≥20 to <40 kg; or
   (iii) 490 mg to a patient weighing ≥40 kg.

In another aspect, the invention features a therapeutic anti-C5 antibody, or antigen-binding fragment thereof, for use in treatment of a pediatric human patient with PNH or aHUS, wherein the patient is complement inhibitor treatment-naïve or eculizumab-experienced, the treatment including administering to the patient during an administration cycle an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
(a) intravenously once on Day 1 of the administration cycle at a dose of:
   (i) 600 mg to a patient weighing ≥10 to <20 kg;
   (ii) 900 mg to a patient weighing ≥20 to <30 kg;
   (iii) 1200 mg to a patient weighing ≥30 to <40 kg;
   (iv) 2400 mg to a patient weighing ≥40 to <60 kg; or
   (v) 2700 mg to a patient weighting ≥60 kg, and
(b) subcutaneously on Day 15 of the administration cycle and every week thereafter at a dose of:
   (i) 150 mg to a patient weighing ≥10 to <20 kg;
   (ii) 245 mg to a patient weighing ≥20 to <40 kg; or
   (iii) 490 mg to a patient weighing ≥40 kg.

In yet another aspect, the invention features the use of a therapeutic anti-C5 antibody, or antigen-binding fragment thereof, in the manufacture of a medicament for treating aHUS or PNH in a pediatric human patient in need thereof, wherein the therapeutic anti-C5 antibody, or antigen-binding fragment thereof includes CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is complement inhibitor treatment-naïve or eculizumab-experienced, and wherein the therapeutic anti-C5 antibody, or antigen-binding fragment thereof, is formulated for administration by:
(a) intravenously once on Day 1 of the administration cycle at a dose of:
   (i) 600 mg to a patient weighing ≥10 to <20 kg;
   (ii) 900 mg to a patient weighing ≥20 to <30 kg;
   (iii) 1200 mg to a patient weighing ≥30 to <40 kg;
   (iv) 2400 mg to a patient weighing ≥40 to <60 kg; or
   (v) 2700 mg to a patient weighting ≥60 kg, and
(b) subcutaneously on Day 15 of the administration cycle and every week thereafter at a dose of:
   (i) 150 mg to a patient weighing ≥10 to <20 kg;
   (ii) 245 mg to a patient weighing ≥20 to <40 kg; or
   (iii) 490 mg to a patient weighing ≥40 kg.

In another aspect, the invention features a method of treating a pediatric human patient with PNH or aHUS, wherein the patient is ravulizumab-experienced, the method including administering to the patient during an administration cycle an effective amount of a therapeutic anti-C5 antibody, or antigen-binding fragment thereof, including CDR1, CDR2, and CDR3 heavy chain sequences as set forth in SEQ ID NOs. 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

In another aspect, the invention features a therapeutic anti-C5 antibody, or antigen-binding fragment thereof, for use in treatment of a pediatric human patient with PNH or aHUS, wherein the patient is ravulizumab-experienced, the treatment including administering to the patient during an administration cycle an effective amount of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
subcutaneously once per week at a dose of:
   (i) 150 mg to a patient weighing ≥10 to <20 kg;
   (ii) 245 mg to a patient weighing ≥20 to <40 kg; or
   (iii) 490 mg to a patient weighing ≥40 kg.

In another aspect, the invention features use of a therapeutic anti-C5 antibody, or antigen-binding fragment thereof, in the manufacture of a medicament for treating aHUS or PNH in a pediatric human patient in need thereof, wherein the therapeutic anti-C5 antibody, or antigen-binding fragment thereof, includes CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ravulizumab-experienced, and wherein the therapeutic anti-C5 antibody, or antigen-binding fragment thereof, is formulated for administration: subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg once per week.

In some embodiments of any of the foregoing aspects, the pediatric human patient is less than 21 years of age. In some embodiments, the pediatric human patient is between 2 and 18 years of age.

In some embodiments of any of the foregoing aspects, the therapeutic anti-C5 antibody, or antigen-binding fragment thereof, is administered subcutaneously to a patient weighing ≥10 to <20 kg at a dose of 150 mg using a syringe in a volume of 1.5 mL. In some embodiments, the 150 mg dose of the therapeutic anti-C5 antibody, or antigen-binding fragment thereof, is formulated at pH 7.4 and a concentration of 100 mg/mL in sodium phosphate, sucrose, L-arginine, PS80, and water for injection.

In some embodiments of any of the foregoing aspects, the therapeutic anti-C5 antibody, or antigen-binding fragment thereof, is administered subcutaneously to a patient weighing ≥20 kg using an OBDS. In some embodiments, the OBDS includes a cartridge including the therapeutic anti-C5 antibody, or antigen-binding fragment thereof, and an OBI. In some embodiments, the therapeutic anti-C5 antibody, or antigen-binding fragment thereof, is administered subcutaneously to a patient weighing ≥20 to <40 kg at a dose of 245 mg using one OBDS. In some embodiments, the 245 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, wherein the OBDS includes one OBI and one pre-filled cartridge containing 245 mg of the therapeutic anti-C5 antibody or antigen-binding fragment thereof.

In some embodiments of any of the foregoing aspects, the therapeutic anti-C5 antibody, or antigen-binding fragment thereof, is administered subcutaneously to a patient weighing ≥40 kg at a dose of 490 mg using two OBDSs. In some embodiments, the 490 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, wherein each OBDS includes one OBI and one pre-filled cartridge containing 245 mg of the therapeutic anti-C5 antibody or antigen-binding fragment thereof.

In some embodiments of any of the foregoing aspects, the OBDS is a single-use electro-mechanical medical device including: an injector including a 29-gauge needle and a cartridge including a piston and telescopic screw assembly. In some embodiments, the cartridge has a volume of 3.5 mL. In some embodiments, the cartridge has a volume of 10 mL. In some embodiments, the OBDS is configured to deliver the therapeutic anti-C5 antibody, or antigen-binding fragment thereof, via subcutaneous injection in about 10 minutes. In some embodiments, the cartridge includes the therapeutic anti-C5 antibody, or antigen-binding fragment thereof, and an opening sealed by a pierceable septum.

In some embodiments of any of the foregoing aspects, the OBDS includes an OBI including:
(i) an injector housing;
(ii) an injection needle translatable between a retracted position, wherein at least a tip of the injection needle is contained within the injector housing, and an injection position, wherein at least the tip of the injection needle protrudes from the injector housing;
(iii) an activation button assembly movably mounted to the injector housing and operatively connected to the injection needle, the activation button assembly being translatable from an unactuated position to an actuated position to drive the injection needle from the retracted position thereof to the injection position thereof;
(iv) a cartridge door movably mounted to the injector housing between an open position and a closed position, the cartridge door including:
   an open end,
   an interior channel having a cartridge mounted therein, the cartridge containing the therapeutic anti-C5 antibody, or antigen-binding fragment thereof, to be dispensed and having an opening at a front end of the cartridge sealed by a pierceable septum and a flange at a rear end thereof, and
   a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and
(v) a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum;
wherein the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum.

In some embodiments of any of the foregoing aspects, the therapeutic anti-C5 antibody, or antigen-binding fragment thereof, is self-administered subcutaneously to the patient. In some embodiments, the therapeutic anti-C5 antibody, or antigen-binding fragment thereof, is administered to a site selected from the patient's arm, abdomen, and thigh. In some embodiments, the therapeutic anti-C5 antibody, or antigen-binding fragment thereof, is administered subcutaneously once weekly after the administration cycle for up to 3 months, 6 months, 9 months, 12 months, 15 months, 18 months, 21 months, two years, or chronically for the remainder of the patient's life.

In some embodiments of any of the foregoing aspects, the patient has previously been treated with eculizumab or a biosimilar thereof or an anti-C5 antibody selected from tesidolumab, crovalimab, CAN106, or pozelimab. In some embodiments, the patient has previously been treated with eculizumab or a biosimilar thereof. In some embodiments, the biosimilar of eculizumab includes ABP 959, ELIZARIA®, SB12, ISU305, ABLYZE®, or BCD 148. In some embodiments, the administration cycle starts about two weeks after the patient's last dose of eculizumab or a biosimilar thereof or an anti-C5 antibody selected from tesidolumab, crovalimab, CAN106, or pozelimab. In some embodiments, the administration cycle starts about two weeks after the patient's last dose of eculizumab or a biosimilar thereof. In some embodiments, the patient has been treated with eculizumab or a biosimilar thereof for at least 90 days prior to Day 1 of the administration cycle. In some embodiments, (i) the patient weighs <20 kg, and the administration cycle starts about 4 weeks after the patient's last dose of ravulizumab or (ii) the patient weighs ≥20 kg, and the administration cycle starts about 8 weeks after the patient's last dose of ravulizumab.

In some embodiments of any of the foregoing aspects, the therapeutic anti-C5 antibody, or antigen-binding fragment thereof, includes a variant human Fc constant region that binds to human neonatal Fc receptor (FcRn), wherein the variant human Fc CH3 constant region includes Met-429-Leu and Asn-435-Ser substitutions at residues corresponding to methionine 428 and asparagine 434 of a native human IgG Fc constant region, each in EU numbering. In some embodiments, the therapeutic anti-C5 antibody, or antigen-binding fragment thereof, includes a heavy chain variable region including the amino acid sequence of SEQ ID NO: 12 and a light chain variable region including the amino acid sequence of SEQ ID NO: 8. In some embodiments, the therapeutic anti-C5 antibody, or antigen-binding fragment thereof, further includes a heavy chain constant region including in the amino acid sequence of SEQ ID NO: 13. In some embodiments, the therapeutic anti-C5 antibody, or antigen-binding fragment thereof, includes a heavy chain polypeptide including the amino acid sequence of SEQ ID NO: 14 and a light chain polypeptide including the amino acid sequence of SEQ ID NO: 11. In some embodiments, the heavy chain variable region includes an N-terminal pyroglutamate residue.

In some embodiments of any of the foregoing aspects, the therapeutic anti-C5 antibody, or antigen-binding fragment thereof, binds to human C5 at pH 7.4 and 25° C. with an affinity dissociation constant (K_{D}) that is in the range 0.1 nM≤K_{D}≤1 nM. In some embodiments, the therapeutic anti-C5 antibody, or antigen-binding fragment thereof, binds to human C5 at pH 6.0 and 25° C. with a K_{D} of ≤10 nM.

In some embodiments of any of the foregoing aspects, the therapeutic anti-C5 antibody is ravulizumab.

In some embodiments of any of the foregoing aspects, the patient is vaccinated against meningococcal infection from serogroups A, C, Y, W135, and B within 3 years prior to, or at least 2 weeks prior to, Day 1 of the administration cycle, and wherein the patient has received vaccination for Streptococcus pneumoniae and Haemophilus influenzae type b.

In some embodiments of any of the foregoing aspects, the patient has PNH. In some embodiments, the patient has PNH confirmed by flow cytometry evaluation of red blood cells (RBCs) and white blood cells (WBCs), with a granulocyte or monocyte clone size of ≥5. In some embodiments, the patient is complement inhibitor treatment-naïve, and has at least one or more of the following symptoms prior to treatment: fatigue, hemoglobinuria, abdominal pain, shortness of breath (dyspnea), anemia, history of a major adverse cardiovascular event (MAVE) (including thrombosis), dysphagia, or erectile dysfunction; or history of packed red blood cell transfusion due to PNH. In some embodiments, (i) the patient is complement inhibitor treatment-naïve, and has lactate dehydrogenase (LDH) ≥1.5× upper limit of normal (ULN) or (ii) the patient is eculizumab- or ravulizumab-experienced, and has LDH ≥1.5×ULN.

In some embodiments of any of the foregoing aspects, the treatment results in: (i) a change in LDH levels relative to baseline, (ii) a reduction in the incidence of breakthrough hemolysis, (iii) achievement of transfusion avoidance, (iv) achievement of stabilized hemoglobin, and/or (v) a change in baseline in PNH RBC clone size. In some embodiments, the treatment results in: (i) a percentage change in LDH levels relative to baseline at Week 10 and 52, (ii) a reduction in the incidence of breakthrough hemolysis through Week 10 and 52, (iii) achievement of transfusion avoidance through Week 10 and 52, (iv) achievement of stabilized hemoglobin through Week 10 and 52, and (v) a change in RBC levels relative to baseline at Week 52.

In some embodiments of any of the foregoing aspects, the patient has aHUS. In some embodiments, the treatment results in: (i) a change in dialysis requirement status, (ii) a change in observed value and change from baseline in estimated glomerular filtration rate (eGFR), (iii) a change in observed value and change from baseline in serum creatinine, and/or (iv) a change in observed value and change from baseline in hematologic parameters selected from (1) platelets, (2) lactate dehydrogenase (LDH), and/or (3) hemoglobin. In some embodiments, the treatment results in: (i) a change in dialysis requirement status relative to baseline through Week 10 and 52, (ii) a change in observed value and change from baseline in eGFR through Week 10 and Week 52, (iii) a change in observed value and change from baseline in serum creatinine through Week 10 and Week 52, and/or (iv) a change in observed value and change from baseline in hematologic parameters selected from (1) platelets, (2) lactate dehydrogenase (LDH), and/or (3) hemoglobin through Week 10 and Week 52. In some embodiments, the patient is not administered immunosuppressive therapies selected from a steroid, a mammalian target of rapamycin (mTOR) inhibitor, or a calcineurin inhibitor unless: (a) as part of an established post-transplant anti-rejection regimen; (b) the patient has confirmed anti-complement factor antibodies requiring immunosuppressive therapy; (c) the steroid is being used treatment of a condition other than aHUS; or (d) the steroid was initiated empirically prior to treatment and is being tapered as standard of care. In some embodiments, the mTOR inhibitor may be sirolimus or everolimus. In some embodiments, the calcineurin inhibitor may be cyclosporine or tacrolimus. In some embodiments, the patient is complement inhibitor treatment-naïve and has evidence of thrombotic microangiopathy (TMA) based on the following prior to the administration cycle: (a) platelet count <150000/μL, (b) LDH≥1.5× upper limit of normal (ULN), (c) hemoglobin lower limit of normal (LLN) for age and sex, and (d) serum creatinine level ≥97.5th percentile for age.

In some embodiments of any of the foregoing aspects, the patient is eculizumab-experienced or ravulizumab-experienced and had the following at the time of a TMA event: (a) increase in LDH >ULN, (b) increase in serum creatinine >ULN, and (c) decrease in platelets <LLN. In some embodiments, the patient is eculizumab-experienced or ravulizumab-experienced and had clinical evidence of response to eculizumab or ravulizumab indicated by stable TMA parameters including: (a) LDH <1.5×ULN, (b) platelet count 150000/μL, and (c) estimated glomerular filtration rate (eGFR) >30 mL/min/1.73 m² using the Schwartz formula. In some embodiments, the patient has a kidney transplant and has: (a) known history of aHUS prior to current kidney transplant, or (b) no known history of aHUS, and persistent evidence of TMA at least 4 days after modifying the immunosuppressive regimen of calcineurin inhibitor or mammalian target of rapamycin inhibitor. In some embodiments, the patient had onset of TMA postpartum, and the patient has persistent symptoms of TMA for greater than 3 days after the day of childbirth. In some embodiments, the treatment results in: (i) an improvement in the patient's dialysis requirement status, (ii) a change in eGFR levels relative to baseline, (iii) a change in serum creatinine levels relative to baseline, (iv) a change in hematologic parameters including platelets, LDH, and/or hemoglobin levels relative to baseline.

In some embodiments of any of the foregoing aspects, the treatment maintains (a) a serum trough concentration of <0.5 μg/mL of free C5 during the administration cycle and/or (b) a serum concentration of 175 μg/mL of the therapeutic anti-C5 antibody, or antigen-binding fragment thereof during the administration cycle. In some embodiments, the method further includes monitoring the concentration of: (a) serum anti-C5 antibody, or antigen-binding fragment thereof, and/or (b) serum free C5. In some embodiments, the concentration of (a) serum anti-C5 antibody, or antigen-binding fragment thereof, and/or (b) serum free C5 is determined from blood samples collected before and after each administration of the therapeutic anti-C5 antibody, or antigen-binding fragment thereof. In some embodiments, the blood samples are collected within 30 minutes prior to the administration and within 60 minutes after each administration of the therapeutic anti-C5 antibody, or antigen-binding fragment thereof.

In some embodiments of any of the foregoing aspects, the treatment results in terminal complement inhibition. In some embodiments, the treatment results in a reduction of hemolysis as assessed by LDH levels. In some embodiments comprises monitoring: (a) the incidence of (i) adverse events (AEs) and serious AEs and (ii) adverse drug events (ADEs) and serious ADEs, (b) the outcome of attempted full-dose administration via an OBI and/or reported device deficiencies or complaints and associated device investigations, and/or (c) the incidence of anti-drug antibodies (ADAs), response categories, and titer. In some embodiments, the treatment produces a change from baseline in patient-reported fatigue, as measured by Pediatric FACIT-Fatigue, optionally wherein the patient is ≥8 years of age. In some embodiments, the treatment produces a change from baseline in PedsQL 4.0 Generic Core Scale.

In another aspect, the invention features a kit for treating PNH or aHUS in a pediatric human patient, wherein the patient is complement inhibitor treatment-naïve or eculizumab-experienced, including: (a) a dose of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, and (b) instructions for using the therapeutic anti-C5 antibody, or antigen-binding fragment thereof, in the method of any of the foregoing aspects.

In some embodiments of the foregoing aspects, the therapeutic anti-C5 antibody, or antigen-binding fragment thereof, is administered:
(a) intravenously once at a dose of:
   (i) 600 mg to a patient weighing ≥10 to <20 kg;
   (ii) 900 mg to a patient weighing ≥20 to <30 kg;
   (iii) 1200 mg to a patient weighing ≥30 to <40 kg;
   (iv) 2400 mg to a patient weighing ≥40 to <60 kg; or
   (v) 2700 mg to a patient weighing ≥60 kg, and
(b) subcutaneously once per week at a dose of:
   (i) 150 mg to a patient weighing ≥10 to <20 kg;
   (ii) 245 mg to a patient weighing ≥20 to <40 kg; or
   (iii) 490 mg to a patient weighing ≥40 kg.

In some embodiments of the foregoing aspects, the therapeutic anti-C5 antibody, or antigen-binding fragment thereof, is provided in a 3 mL vial including Type I borosilicate glass for (a) intravenous administration and (b) subcutaneous administration to a patient weighing ≥10 to <20 kg.

In another aspect, the invention features a kit for treating PNH or aHUS in a pediatric human patient, wherein the patient is ravulizumab-experienced, including: (a) a dose of a therapeutic anti-C5 antibody, or antigen-binding fragment thereof, including CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, and (b) instructions for using the therapeutic anti-C5 antibody, or antigen-binding fragment thereof, in the method of any of the foregoing aspects.

In some embodiments of the foregoing aspects, the therapeutic anti-C5 antibody, or antigen-binding fragment thereof, is administered subcutaneously once per week at a dose of: (i) 150 mg to a patient weighing ≥10 to <20 kg, (ii) 245 mg to a patient weighing ≥20 to <40 kg, or (iii) 490 mg to a patient weighing ≥40 kg. In some embodiments, the pediatric human patient is less than 21 years of age. In some embodiments, the pediatric human patient is between 2 and 18 years of age. In some embodiments, the therapeutic anti-C5 antibody, or antigen-binding fragment thereof, is administered to a site selected from the patient's arm, abdomen, and thigh.

Further, the disclosure encompasses any of the above embodiments being used with any other of the above embodiments in any combination.

### BRIEF DESCRIPTION OF THE DRAWINGS

FIG. 1 is a schematic diagram depicting the design of a Phase III clinical trial in complement inhibitor treatment-naïve or eculizumab-experienced pediatric patients having PNH or aHUS.

FIG. 2 is a schematic diagram depicting the design of a Phase III clinical trial in ravulizumab-experienced pediatric patients having PNH or aHUS.

FIG. 3 is a flowchart depicting criteria for categorizing medical device deficiencies and complaints. Abbreviations: ADE=adverse device effect; AE=adverse event; SADE=serious adverse device effect.

FIG. 4 is a flowchart depicting criteria for determining whether an adverse event, adverse device effect, serious adverse event, or serious adverse device effect have occurred. Abbreviations: AE=adverse event; ADE=adverse device effect; SAE=serious adverse event; SADE=serious adverse device effect.

FIG. 5 depicts the Pediatric Functional Assessment of Chronic Illness Therapy—Fatigue. A prompt to the patient is as follows: This figure shows a list of statements that other people with your illness have said are important. Please circle or mark one number per line to indicate your response as it applies to the past 7 days.

FIGS. 6A and 6B depict the Pediatric Quality of Life Inventory (PedsQL) 4.0 Generic Core Scale Parent Report for Toddlers (Ages 2-4).

FIGS. 7A-7C depict the PedsQL 4.0 Generic Core Scale Young Child Report (Ages 5-7) FIGS. 8A and 8B depict the PedsQL 4.0 Generic Core Scale Parent Report for Young Children (Ages 5-7).

FIGS. 9A and 9B depict the PedsQL 4.0 Generic Core Scale Child Report (Ages 8-12).

FIGS. 10A and 10B depict the PedsQL 4.0 Generic Core Scale Parent Report for Children (Ages 8-12).

FIGS. 11A and 11B depict the PedsQL 4.0 Generic Core Scale Teen Report (Ages 13-18).

FIGS. 12A and 12B depict the PedsQL 4.0 Generic Core Scale Parent Report for Teens (Ages 13-18).

### DETAILED DESCRIPTION

### Definitions

As used herein, the term “subject” or “patient” is a human patient (e.g., a patient having Paroxysmal Nocturnal Hemoglobinuria (PNH)) or atypical hemolytic uremic syndrome (aHUS)). As used herein, the terms “subject” and “patient” are interchangeable. In some examples, the patient is a pediatric patient (e.g., a patient of less than 21 years of age, e.g., a patient between 2 and 18 (e.g., 2, 3, 4, 5, 6, 7, 8, 9, 10, 11, 12, 13, 14, 15, 16, 17, or 18) years of age).

As used herein, the phrase “complement inhibitor-naïve” encompasses a pediatric human patient who has not received therapy with any complement inhibitor (e.g., an anti-C5 antibody) prior to administration of an anti-C5 antibody (e.g., ravulizumab) in accordance with the devices, compositions, and methods described herein.

As used herein, the phrase “eculizumab-experienced” encompasses a pediatric human patient who has received therapy with an anti-C5 antibody (e.g., eculizumab, a biosimilar thereof, or another non-ravulizumab anti-C5 antibody) prior to administration of an anti-C5 antibody in accordance with the devices, compositions, and methods described herein. In some embodiments, a patient is eculizumab-experienced if the patient has received a therapy including eculizumab or a biosimilar thereof, or an anti-C5 antibody selected from tesidolumab, crovalimab, CAN106, or pozelimab, prior to administration of an anti-C5 antibody (e.g., ravulizumab) in accordance with the devices, compositions, and methods described herein. In some embodiments, the biosimilar of eculizumab includes ABP 959, ELIZARIA®, SB12, ISU305, ABLYZE®, or BCD 148.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of PNH (e.g., fatigue, abdominal pain, dyspnea, dysphagia, chest pain, or erectile dysfunction) or at least one symptom of aHUS (e.g., severe hypertension, proteinuria, uremia, lethargy/fatigue, irritability, thrombocytopenia, microangiopathic hemolytic anemia, and renal function impairment (e.g., acute renal failure)).

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of anti-C5 antibody, or antigen-binding fragment thereof, clinically proven to alleviate at least one symptom of PNH (e.g., fatigue, abdominal pain, dyspnea, dysphagia, chest pain, or erectile dysfunction) or at least one symptom of aHUS (e.g., severe hypertension, proteinuria, uremia, lethargy/fatigue, irritability, thrombocytopenia, microangiopathic hemolytic anemia, and renal function impairment (e.g., acute renal failure)). An effective amount can be administered in one or more administrations.

As used herein, the terms “fixed dose,” “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-C5 antibody, or antigen-binding fragment thereof).

As used herein, the term “loading dose” refers to the initial dose administered to the patient. A loading may be, for example, 600 mg, 900 mg, 1200 mg, 2400 mg, or 2700 mg. Loading doses may be titered based on body weight.

As used herein, the term “maintenance dose” refers to a dose administered to the patient after the loading dose. For example, a maintenance dose may be 150 mg, 245 mg, or 490 mg. Maintenance doses may be titered based on body weight.

As used herein, the terms “on-body delivery system” and “OBDS” refer to a wearable device for subcutaneous administration of a therapeutic agent (e.g., an anti-C5 antibody (e.g., ravulizumab) as described herein). In some examples, an OBDS may include an on-body injector (OBI) and a cartridge (e.g., a pre-filled cartridge). An exemplary device includes the OBDS manufactured by West Pharmaceuticals, Inc., which is currently approved for use with evolocumab (Repatha®) as a combination agent in the United States and CE marked in the European Union as a class IIA Medical Device. The device is a compact, sterile, single-use, disposable, electro-mechanical (battery powered, microprocessor controlled), investigational medical device with a 29-gauge integrated needle (manufactured by West Pharmaceuticals, Inc.) designed to be used together with a prefilled stoppered Crystal Zenith® cartridge with a piston and telescopic screw assembly (TSA). Exemplary OBDS devices include the West SMARTDOSE® OBDS (which may be referred to as a West SMARTDOSE® generation I device) and SMARTDOSE®10 OBDS (which may be referred to as a West SMARTDOSE® generation II device). Exemplary OBDS devices are described in U.S. Patent Publication No. US2020/0254185 and U.S. Pat. No. 11,571,517, which are incorporated herein by reference in their entirety. In some embodiments, the cartridge has a volume of 3.5 mL. In some embodiments, the cartridge has a volume of 10 mL.

As used herein, the terms “on-body injector” and “OBI” refer to a wearable injector, which may be a component of an OBDS as described herein. An OBI may include, e.g., an injection needle, which may be configured for subcutaneous administration of a therapeutic agent (e.g., an anti-C5 antibody (e.g., ravulizumab) as described herein). An OBI may be battery-powered and include means for accepting a cartridge (e.g., a pre-filled cartridge) containing a therapeutic agent a therapeutic agent (e.g., an anti-C5 antibody (e.g., ravulizumab) as described herein).

As used herein, the phrase “ravulizumab-experienced” encompasses a pediatric human patient who has received therapy with ravulizumab prior to administration of an anti-C5 antibody in accordance with the devices, compositions, and methods described herein.

As used herein, the term “serum trough level” refers to the lowest level that an agent (e.g., the anti-C5 antibody, or antigen-binding fragment thereof) is present in the serum. In contrast, a “peak serum level”, refers to the highest level of the agent in the serum. The “average serum level”, refers to the mean level of the agent in the serum over time.

In one embodiment, the treatment regimens described are sufficient to maintain particular serum trough concentrations of the anti-C5 antibody or antigen-binding fragment thereof. In one embodiment, for example, the treatment maintains a serum trough concentration of the anti-C5 antibody or antigen-binding fragment thereof, of 50, 55, 60, 65, 70, 75, 80, 85, 90, 95, 100, 105, 110, 115, 120, 125, 130, 135,140, 145, 150, 155, 160,165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment maintains a serum trough concentration of the anti-C5 antibody or antigen-binding fragment thereof of 100 μg/mL or greater. In another embodiment, the treatment maintains a serum trough concentration of the anti-C5 antibody or antigen-binding fragment thereof of 150 μg/mL or greater. In another embodiment, the treatment maintains a serum trough concentration of the anti-C5 antibody or antigen-binding fragment thereof of 200 μg/mL or greater. In another embodiment, the treatment maintains a serum trough concentration of the anti-C5 antibody or antigen-binding fragment thereof of 250 μg/mL or greater. In another embodiment, the treatment maintains a serum trough concentration of the anti-C5 antibody or antigen-binding fragment thereof of 300 μg/mL or greater. In another embodiment, the treatment maintains a serum trough concentration of the anti-C5 antibody or antigen-binding fragment thereof of between 100 μg/mL and 200 μg/mL. In another embodiment, the treatment maintains a serum trough concentration of the anti-C5 antibody or antigen-binding fragment thereof of about 175 μg/mL.

In another embodiment, to obtain an effective response, the anti-C5 antibody or antigen-binding fragment thereof is administered to a patient in an amount and with a frequency to maintain a desired minimum free C5 concentration. In one embodiment, for example, the anti-C5 antibody or antigen-binding fragment thereof is administered to the patient in an amount and with a frequency to maintain a free C5 concentration of 0.2 pg/mL, 0.3 pg/mL, 0.4 pg/mL, 0.5 pg/mL or less. In another embodiment, the anti-C5 antibody or antigen-binding fragment thereof is administered to the patient in an amount and with a frequency to maintain a free C5 concentration of 0.309 to 0.5 pg/mL or less. In another embodiment, the treatment described herein reduces free C5 concentration by greater than 99% throughout the treatment period. In another embodiment, the treatment reduces free C5 concentration greater than 99.5% throughout the treatment period.

The term “antibody” describes polypeptides including at least one antibody derived antigen binding site (e.g., VH/VL region or Fv, or CDR). Antibodies include known forms of antibodies. The antibody can be, for example, a human antibody, a humanized antibody, a bispecific antibody, a chimeric antibody or a camelid antibody. The antibody also can be a Fab, Fab′2, scFv, SMIP, Affibody®, nanobody or a single domain antibody. The antibody also can be of any of the following isotypes: IgG1, IgG2, IgG3, IgG4, IgM, IgA1, IgA2, IgAsec, IgD, and IgE, and hybrid isotypes, e.g., IgG2/4. The antibody may be a naturally occurring antibody or may be an antibody that has been altered by a protein engineering technique (e.g., by mutation, deletion, substitution, conjugation to a non-antibody moiety). An antibody may include, for example, one or more variant amino acids (compared to a naturally occurring antibody), which changes a property (e.g., a functional property) of the antibody. Numerous such alterations are known in the art that affect, e.g., half-life, effector function, and/or immune responses to the antibody in a patient. The term antibody also includes artificial or engineered polypeptide constructs that include at least one antibody-derived antigen binding site.

### Methods of Use

The methods and compositions described herein are useful for treating paroxysmal nocturnal hemoglobinuria (PNH) and atypical hemolytic uremic syndrome (aHUS), which are ultra-rare disorders driven by chronic uncontrolled complement activation, in pediatric human patients. In each case, ongoing complement dysregulation leads to increased activation of C5 systemically with consequent terminal complement activation, resulting in the devastating clinical manifestations of these disorders. Patients with PNH or aHUS are at risk of substantial morbidity and mortality.

The disease begins with the clonal expansion of a hematopoietic stem cell that has acquired a somatic mutation in the PIGA gene (Brodsky R A., Blood. 2014; 124:2804-1). Consequently, PNH blood cells lack the glycophosphatidylinositol (GPI) anchor protein and are deficient in the membrane-bound complement inhibitory proteins CD55 and CD59. In the absence of CD55, there is increased deposition of complement protein C3 cleavage products on blood cell membrane surfaces, in turn leading to cleavage of C5 into C5a and C5b. The pathology and clinical presentations in patients with PNH are driven by uncontrolled terminal complement activation.

A substantial proportion of patients with PNH experience renal dysfunction and pulmonary hypertension (Hillmen, et al., Am J Hematol. 2010; 85:553-9. [erratum in Am J Hematol. 2010; 85:911.]; Hill, et al., Br. J Haematol. 2012; 158:409-14.; Hill, et al., Blood 2013; 121:4985-96). Patients also experience venous or arterial thrombosis in diverse sites, including the abdomen or central nervous system (Brodsky R A., Blood. 2014; 124:2804-1).

The pathology and clinical presentations of patients with aHUS are also driven by terminal complement activation. More specifically, activation of C5 and dysregulation of complement activation lead to endothelial damage, platelet consumption, and thrombotic microangiopathic (TMA) events, characterized by thrombocytopenia, mechanical intravascular hemolysis, and kidney injury. Importantly, approximately 20% of patients experience extra-renal manifestations of disease as well, including central nervous system, cardiac, gastrointestinal, distal extremities, and severe systemic organ involvement (Loirat, et al., Orphanet. J. Rare Dis. 2011; 6:60). Symptoms of aHUS are well-known to those of skill in the art of rare disease or kidney disease medicine and include, e g., severe hypertension, proteinuria, uremia, lethargy/fatigue, irritability, thrombocytopenia, microangiopathic hemolytic anemia, and renal function impairment (e.g., acute renal failure).

aHUS can be genetic, acquired, or idiopathic. aHUS can be considered genetic when two or more (e.g., three, four, five, or six or more) members of the same family are affected by the disease at least six months apart and exposure to a common triggering agent has been excluded, or when one or more aHUS-associated gene mutations (e.g., one or more mutations in CFH, MCP/CD46, CFB, or CFI) are identified in a subject. For example, a subject can have CFH-associated aHUS, CFB-associated aHUS, CFI-associated aHUS, or MCP-associated aHUS. Up to 30% of genetic aHUS is associated with mutations in CFH, 12% with mutations in MCP, 5-10% with mutations in CFI, and less than 2% with mutations in CFB. Genetic aHUS can be multiplex (i.e., familial; two or more affected family members) or simplex (i.e., a single occurrence in a family). aHUS can be considered acquired when an underlying environmental factor (e.g., a drug, systemic disease, or viral or bacterial agents that do not result in Shiga-like exotoxins) or trigger can be identified. aHUS can be considered idiopathic when no trigger (genetic or environmental) is evident.

Laboratory tests can be performed to determine whether a human subject has thrombocytopenia, microangiopathic hemolytic anemia, or acute renal insufficiency. Thrombocytopenia can be diagnosed by a medical professional as one or more of: (i) a platelet count that is less than 150,000/mm³ (e.g., less than 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than 10 mg/dL (e.g., less than 6.5 mg/dL); (ii) increased serum lactate dehydrogenase (LDH) concentrations (>460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668). Blood concentrations of C3 and C4 can also be used as a measure of complement activation or dysregulation. In addition, a subject's condition can be further characterized by identifying the subject as harboring one or more mutations in a gene associated with aHUS such as CFI, CFB, CFH, or MCP (supra). Suitable methods for detecting a mutation in a gene include, e.g., DNA sequencing and nucleic acid array techniques. See, e.g., Breslin et al. (2006) Clin Am Soc Nephrol 1:88-99 and Goicoechea de Jorge et al. (2007) Proc Natl Acad Sci USA 104:240-245.

Provided herein is a method of treating a pediatric human patient with PNH or aHUS, wherein the patient is complement inhibitor treatment-naïve or eculizumab-experienced, the method including administering to the patient during an administration cycle an effective amount of an anti-C5 antibody, or antigen-binding fragment thereof, wherein the anti-C5 antibody, or antigen-binding fragment thereof, is administered:
(a) intravenously once on Day 1 of the administration cycle at a dose of:
   (i) 600 mg to a patient weighing ≥10 to <20 kg;
   (ii) 900 mg to a patient weighing ≥20 to <30 kg;
   (iii) 1200 mg to a patient weighing ≥30 to <40 kg;
   (iv) 2400 mg to a patient weighing ≥40 to <60 kg; or
   (v) 2700 mg to a patient weighing ≥60 kg, and
(b) subcutaneously on Day 15 of the administration cycle and every week thereafter at a dose of:
   (i) 150 mg to a patient weighing ≥10 to <20 kg;
   (ii) 245 mg to a patient weighing ≥20 to <40 kg; or
   (iii) 490 mg to a patient weighing ≥40 kg.

Additionally, provided herein is a method of treating a pediatric human patient with PNH or aHUS, wherein the patient is ravulizumab-experienced, the method including administering to the patient during an administration cycle an effective amount of an anti-C5 antibody, or antigen-binding fragment thereof, wherein the anti-C5 antibody, or antigen-binding fragment thereof, is administered subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

In some embodiments, the pediatric human patient is less than 21 years of age (e.g., 20, 19, 18, 17, 16, 15, 10, 5, or 2 years of age). In some embodiments, the pediatric human patient between 2 and 18 years of age (e.g., 2, 3, 4, 5, 6, 7, 8, 9, 10, 11, 12, 13, 14, 15, 16, 17, or 18 years of age).

Any suitable anti-C5 antibody, or antigen-binding fragment thereof, may be used, including any suitable anti-C5 antibody or antigen-binding fragment thereof disclosed herein. In some embodiments, the anti-C5 antibody, or antigen-binding fragment thereof, comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6. In some embodiments, the anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering. In some embodiments, the anti-C5 antibody, or antigen-binding fragment thereof, comprises a heavy chain variable region comprising the amino acid sequence of SEQ ID NO: 12 and a light chain variable region comprising the amino acid sequence of SEQ ID NO: 8. In some embodiments, the heavy chain variable region comprises an N-terminal pyroglutamate residue. In some embodiments, the anti-C5 antibody, or antigen-binding fragment thereof, further comprises a heavy chain constant region comprising the amino acid sequence of SEQ ID NO: 13. In some embodiments, the anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.

In some embodiments, the anti-C5 antibody, or antigen-binding fragment thereof, is administered subcutaneously to a patient weighing ≥10 to <20 kg at a dose of 150 mg using a syringe in a volume of 1.5 mL. In some embodiments, the 150 mg dose of the anti-C5 antibody, or antigen-binding fragment thereof, is formulated at pH 7.4 and a concentration of 100 mg/mL in sodium phosphate, sucrose, L-arginine, polysorbate 80 (PS80), and water for injection.

In some embodiments, the anti-C5 antibody, or antigen-binding fragment thereof, is administered subcutaneously to a patient weighing ≥20 kg using an on-body delivery system (OBDS). In some embodiments, the OBDS is a single-use electro-mechanical medical device comprising an injector comprising a 29-gauge needle and a cartridge comprising a piston and telescopic screw assembly. In some embodiments, the OBDS is configured to deliver the anti-C5 antibody, or antigen-binding fragment thereof, via subcutaneous injection in about 10 minutes. In some embodiments, the OBDS comprises a cartridge comprising the anti-C5 antibody, or antigen-binding fragment thereof, and an on-body injector (OBI). In some embodiments, the anti-C5 antibody, or antigen-binding fragment thereof, is administered subcutaneously to a patient weighing ≥20 to <40 kg at a dose of 245 mg using one OBDS. In some embodiments, the 245 mg dose of the anti-C5 antibody, or antigen-binding fragment thereof, is formulated at pH 7.4 and a concentration of 70 mg/mL in 50 mM sodium phosphate, 25 mM L-arginine, 5% weight by volume (w/v) sucrose, 0.05% w/v PS80, and water for injection, and wherein the OBDS comprises one OBI and one pre-filled cartridge containing 245 mg of the anti-C5 antibody or antigen-binding fragment thereof.

In some embodiments, the anti-C5 antibody, or antigen-binding fragment thereof, is administered subcutaneously to a patient weighing ≥40 kg at a dose of 490 mg using two OBDSs. In some embodiments, the 490 mg dose of the anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein each OBDS comprises one OBI and one pre-filled cartridge containing 245 mg of the anti-C5 antibody or antigen-binding fragment thereof.

In some embodiments, the anti-C5 antibody, or antigen-binding fragment thereof, is self-administered subcutaneously. In some embodiments, the anti-C5 antibody, or antigen-binding fragment thereof, is administered to a site selected from the patient's arm, abdomen, and thigh. In some embodiments, the anti-C5 antibody, or antigen-binding fragment thereof, is administered subcutaneously once weekly after the administration cycle for up to 3 months, 6 months, 9 months, 12 months, 15 months, 18 months, 21 months, two years, or chronically for the remainder of the patient's life.

In some embodiments, the patient has previously been treated with eculizumab or a biosimilar thereof or an anti-C5 antibody selected from tesidolumab, crovalimab, CAN106, or pozelimab. In some embodiments, the administration cycle starts about two weeks after the patient's last dose of eculizumab or a biosimilar thereof or an anti-C5 antibody selected from tesidolumab, crovalimab, CAN106, or pozelimab. In some embodiments, the patient has previously been treated with eculizumab or a biosimilar thereof. In some embodiments, the administration cycle starts about two weeks after the patient's last dose of eculizumab or a biosimilar thereof. In some embodiments, the patient has been treated with eculizumab or a biosimilar thereof for at least 90 days prior to Day 1 of the administration cycle.

In some embodiments, (i) the patient weighs <20 kg, and the administration cycle starts about 4 weeks after the patient's last dose of ravulizumab, or (ii) the patient weighs ≥20 kg, and the administration cycle starts about 8 weeks after the patient's last dose of ravulizumab. In some embodiments, the patient is vaccinated against meningococcal infection from serogroups A, C, Y, W135, and B within 3 years prior to, or at least 2 weeks prior to, Day 1 of the administration cycle, and wherein the patient has received vaccination for Streptococcus pneumoniae and Haemophilus influenzae type b.

In some embodiments, the patient has PNH. In some embodiments, the patient has PNH confirmed by flow cytometry evaluation of red blood cells (RBCs) and white blood cells (WBCs), with a granulocyte or monocyte clone size of ≥5. In some embodiments, the patient is complement inhibitor treatment-naïve, and has at least one or more of the following symptoms prior to treatment: fatigue, hemoglobinuria, abdominal pain, shortness of breath (dyspnea), anemia, history of a major adverse cardiovascular event (MAVE) (including thrombosis), dysphagia, or erectile dysfunction; or history of packed red blood cell transfusion due to PNH. In some embodiments, the patient is complement inhibitor treatment-naïve, and has lactate dehydrogenase (LDH) ≥1.5× upper limit of normal (ULN). In some embodiments, the patient is eculizumab- or ravulizumab-experienced, and has LDH ≥1.5×ULN.

In some embodiments, the patient has aHUS. In some embodiments, the patient is not administered an immunosuppressive therapy comprising a steroid, a mammalian target of rapamycin (mTOR) inhibitor, or a calcineurin inhibitor unless: (a) as part of an established post-transplant anti-rejection regimen, (b) the patient has confirmed anti-complement factor antibodies requiring immunosuppressive therapy, (c) the steroid is being used for treatment of a condition other than aHUS, or (d) the steroid was initiated empirically prior to treatment and is being tapered as standard of care. In some embodiments, the patient is complement inhibitor treatment-naïve and has evidence of TMA based on the following prior to the administration cycle: (a) platelet count <150000/μL, (b) LDH ≥1.5× upper limit of normal (ULN), (c) hemoglobin lower limit of normal (LLN) for age and sex, and (d) serum creatinine level ≥97.5th percentile for age. In some embodiments, the patient is eculizumab-experienced or ravulizumab-experienced and had clinical evidence of response to eculizumab or ravulizumab indicated by stable TMA parameters comprising: (a) LDH <1.5×ULN, (b) platelet count ≥150000/μL, and (c) estimated glomerular filtration rate (eGFR) >30 mL/min/1.73 m² using the Schwartz formula. In some embodiments, the patient has a kidney transplant and has: (a) known history of aHUS prior to current kidney transplant or (b) no known history of aHUS, and persistent evidence of TMA at least 4 days after modifying the immunosuppressive regimen of calcineurin inhibitor or mammalian target of rapamycin inhibitor. In some embodiments, the patient had onset of TMA postpartum, and the patient has persistent symptoms of TMA for greater than 3 days after the day of childbirth.

### Delivery Systems

Subcutaneous administration of an anti-C5 antibody, or antigen-binding fragment thereof, (e.g., ravulizumab) according to the methods described herein can be accomplished by any suitable means. In addition, the anti-C5 antibody, or antigen-binding fragment thereof, can be administered subcutaneously by a medical professional or self-administered.

In one embodiment, the anti-C5 antibody, or antigen-binding fragment thereof, is administered subcutaneously using an OBDS. Any suitable OBDS may be used.

An exemplary OBDS for subcutaneous administration of an anti-C5 antibody, or antigen-binding fragment thereof, includes the OBDS manufactured by West Pharmaceuticals, Inc., which is currently approved for use with evolocumab (Repatha®) as a combination agent in the United States and CE marked in the European Union as a class IIA Medical Device. The device is a compact, sterile, single-use, disposable, electro-mechanical (battery powered, microprocessor controlled), investigational medical device with a 29-gauge integrated needle (manufactured by West Pharmaceuticals, Inc.) designed to be used together with a prefilled stoppered Crystal Zenith® cartridge with a piston and telescopic screw assembly (TSA). Exemplary OBDS devices include the West SMARTDOSE® OBDS (which may be referred to as a West SMARTDOSE® generation I device) and SMARTDOSE® 10 OBDS (which may be referred to as a West SMARTDOSE® generation II device). Exemplary OBDS devices are described in U.S. Patent Publication No. US2020/0254185 and U.S. Pat. No. 11,571,517, which are incorporated herein by reference in their entirety.

The cartridge may have any suitable volume. In some embodiments, the cartridge has a volume of 3.5 mL. In some embodiments, the cartridge has a volume of 10 mL.

In some embodiments, provided herein is an OBDS configured for administration of an effective amount of an anti-C5 antibody, or antigen-binding fragment thereof, to treat paroxysmal nocturnal hemoglobinuria (PNH) or atypical hemolytic uremic syndrome (aHUS) in a pediatric human patient in need thereof, wherein the anti-C5 antibody, or antigen-binding fragment thereof, is administered (a) subcutaneously once per week at a dose of: (i) 245 mg to a patient weighing ≥20 to <40 kg; or (ii) 490 mg to a patient weighing ≥40 kg. In some embodiments, provided herein is an OBDS configured for treating PNH or aHUS in a pediatric human patient in need thereof, wherein the OBDS comprises an effective amount of a therapeutic anti-C5 antibody, or antigen-binding fragment thereof, and wherein the OBDS comprises means for subcutaneous administration of 245 mg to a patient weighing ≥20 to <40 kg or 490 mg to a patient weighing ≥40 kg.

In some embodiments, the patient is complement inhibitor treatment-naïve or eculizumab-experienced. In some embodiments, the patient is ravulizumab-experienced.

In some embodiments, the patient is less than 21 years of age (e.g., 20, 19, 18, 17, 16, 15, 10, 5, or 2 years of age). In some embodiments, the patient is between 2 and 18 years of age (e.g., 2, 3, 4, 5, 6, 7, 8, 9, 10, 11, 12, 13, 14, 15, 16, 17, or 18 years of age).

In some embodiments, the OBDS is configured for administration of the therapeutic anti-C5 antibody, or antigen-binding fragment thereof, subcutaneously to a patient weighing ≥20 to <40 kg at a dose of 245 mg using one OBDS. In some embodiments, the 245 mg dose of the anti-C5 antibody, or antigen-binding fragment thereof, is formulated at pH 7.4 and a concentration of 70 mg/mL in 50 mM sodium phosphate, 25 mM L-arginine, 5% weight by volume (w/v) sucrose, 0.05% w/v polysorbate 80 (PS80), and water for injection, and wherein the OBDS comprises one OBI and one pre-filled cartridge containing 245 mg of the anti-C5 antibody or antigen-binding fragment thereof.

In some embodiments, the OBDS is configured for administration of the anti-C5 antibody, or antigen-binding fragment thereof, subcutaneously to a patient weighing ≥40 kg at a dose of 490 mg in combination with a second OBDS. In some embodiments, the 490 mg dose of the anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein each OBDS comprises one OBI and one pre-filled cartridge containing 245 mg of the anti-C5 antibody or antigen-binding fragment thereof.

In some embodiments, the OBDS is a single-use electro-mechanical medical device comprising: an injector comprising a 29-gauge needle and a cartridge comprising a piston and telescopic screw assembly. In some embodiments, the cartridge has a volume of 3.5 mL. In some embodiments, the OBDS is configured to deliver the anti-C5 antibody, or antigen-binding fragment thereof, via subcutaneous injection in about 10 minutes. In some embodiments, the cartridge comprises the anti-C5 antibody, or antigen-binding fragment thereof, and an opening sealed by a pierceable septum.

Exemplary, non-limiting OBDS devices that may be used are described in U.S. Pat. No. 11,571,517 and U.S. Patent Publication No. US2020/0254185, which are incorporated herein by reference in their entirety.

In some embodiments, the OBDS comprises an OBI. An OBI may include, e.g., an injection needle, which may be configured for subcutaneous administration of a therapeutic agent (e.g., an anti-C5 antibody (e.g., ravulizumab) as described herein). An OBI may be battery-powered and include means for accepting a cartridge (e.g., a pre-filled cartridge) containing a therapeutic agent a therapeutic agent (e.g., an anti-C5 antibody (e.g., ravulizumab) as described herein). In some embodiments, the OBI is configured to maintain a cartridge loaded therein in a sealed state until the OBDS is activated for use.

For example, provided herein is provided an injector (e.g., an OBI) that includes an injector housing; an injection needle translatable between a retracted position, wherein at least a tip of the injection needle is contained within the injector housing, and an injection position, wherein at least the tip of the injection needle protrudes from the injector housing; an activation button assembly movably mounted to the injector housing and operatively connected to the injection needle, the activation button assembly being translatable from an unactuated position to an actuated position to drive the injection needle from the retracted position thereof to the injection position thereof; a cartridge door movably mounted to the injector housing between an open position and a closed position, the cartridge door that includes: an open end, an interior channel having a cartridge mounted therein, the cartridge containing a substance to be dispensed and having an opening at a front end of the cartridge sealed by a pierceable septum and a flange at a rear end thereof, and a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum; wherein: the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum; and wherein the substance includes an anti-C5 antibody, or antigen-binding fragment thereof.

In some embodiments, the OBI further includes a driving assembly engageable with the cartridge to expel the substance therefrom, the driving assembly being operatively engaged with the activation button assembly and being configured to drive the cartridge from the sealed position to the unsealed position upon movement of the activation button assembly from the unactuated position to the actuated position. In some embodiments, the deflectable interference member includes a cantilevered arm.

In some embodiments, the cantilevered arm defines a first end connected to the cartridge door and extends to a second, free end proximate the open end of the cartridge door.

In some embodiments, the cartridge door includes a sidewall and the cantilevered arm defines a deflectable portion of the cartridge door sidewall.

In some embodiments, the sidewall of the cartridge door defines a first internal perimeter at the second, free end of the cantilevered arm when the cantilevered arm is in the resting position thereof, and the sidewall of the cartridge door defines a second internal perimeter at the second, free end of the cantilevered arm when the cantilevered arm is deflected from the resting position thereof, the first internal perimeter being smaller than a largest outer perimeter of the cartridge insertable into the interior channel, thereby limiting the insertion depth of the cartridge into the interior channel of the cartridge door, and the second internal perimeter being greater than the largest outer perimeter of the cartridge, thereby enabling further advancement of the cartridge into the interior channel of the cartridge door.

In some embodiments, the activation button assembly includes a post extending therefrom to a terminal end having a tapered face, the post having a post pathway along which the post travels from the unactuated position of the activation button assembly to the actuated position of the activation button assembly, and wherein the cantilevered arm includes a tab laterally extending therefrom toward the post pathway, the tab having a hooked end facing the tapered face of the post, wherein movement of the activation button assembly from the unactuated position to the actuated position, in the closed position of the cartridge door, engages the post with the tab, the tapered face engaging and laterally translating the hooked end, and, in turn, deflecting the cantilevered arm away from the resting position thereof.

In some embodiments, the OBI further includes a biasing member connected with the activation button assembly and the injection needle, the biasing member being stabilized in a stored energy state in the unactuated position of the activation button assembly, and released in the actuated position of the activation button assembly into an energy releasing state to drive the injection needle from the retracted position thereof to the injection position thereof, wherein: a position of the activation button assembly between the unactuated position thereof and the actuated position thereof defines a threshold point, and movement of the activation button assembly beyond the threshold point secures the activation button assembly in the actuated position and the injection needle in the injection position.

In some embodiments, the biasing member biases the activation button assembly into the unactuated position thereof and returns the activation button assembly to the unactuated position thereof in response to movement of the activation button assembly not surpassing the threshold point.

In some embodiments, the OBI further includes a needle hub movably mounted within the injector housing, the injection needle being supported by the movable needle hub and the needle hub and the injection needle being translatable between the retracted position and the injection position.

In some embodiments, the OBI further includes an elongate first post connected with the injector housing and projecting upwardly therefrom, the elongate first post having an upper end including a downwardly inclined surface and an undercut underlying the inclined surface; a deflectable second post connected with the injector housing and projecting upwardly therefrom, the deflectable second post including a flange supporting a portion of the needle hub thereon, thereby securing the needle hub and the injection needle in the retracted position thereof; and the activation button assembly includes a downwardly projecting first arm having a flanged terminal end slidable along the inclined surface of the elongate first post, the first arm being elastically deflectable from an original state thereof; wherein: movement of the activation button assembly slides the flanged terminal end of the first arm along the inclined surface of the first post, thereby elastically deflecting the first arm away from the original state thereof; the inclined surface and the undercut of the elongate first post meet at a vertex defining the threshold point, and movement of the activation button assembly beyond the vertex triggers retraction of the first arm back toward the original state thereof, hooking the flanged terminal end thereof onto the undercut of the elongate first post and securing the activation button assembly in the actuated position thereof, and movement of the activation button beyond the vertex also engages the flanged terminal end of the first arm with the second post and deflects the second post, whereby the deflected second post releases the needle hub, and, in turn, releases the biasing member into the energy releasing state to drive the needle hub and the injection needle from the retracted position thereof to the injection position thereof.

In some embodiments, the injector (e.g., the OBI) may be configured to receive a cartridge containing a substance (e.g., as an anti-C5 antibody, or antigen-binding fragment thereof, described herein) to be dispensed, the cartridge having an opening sealed by a pierceable septum, the injector comprising: an injector housing; an activation button assembly movably mounted to the injector housing, the activation button assembly being translatable from an unactuated position to an actuated position; an injection needle translatable between a retracted position, where at least a tip of the injection needle is contained within the injector housing, and an injection position, where at least the tip of the injection needle protrudes from the injector housing; a cartridge door movably mounted to the injector housing between an open position and a closed position, the cartridge door comprising: an open end, an interior channel configured to receive the cartridge, and a cartridge piercing needle mounted within the interior channel and fluidly connected with the injection needle; and an interference member having a resting position configured to limit an insertion depth of the cartridge in the interior channel of the cartridge door to a sealed position, wherein the cartridge piercing needle does not fully penetrate the pierceable septum in the sealed position, wherein: the cartridge door is movable to the closed position when the cartridge is in the sealed position, and wherein movement of the activation button assembly from the unactuated position to the actuated position, when the cartridge door is in the closed position, deflects the interference member out of the resting position, thereby enabling advancement of the cartridge into the interior channel of the cartridge door to an unsealed position, wherein the cartridge piercing needle fully penetrates the pierceable septum in the unsealed position to dispense the substance (e.g., an anti-C5 antibody, or antigen-binding fragment thereof) through the injection needle.

In some embodiments, the injector further comprises a driving assembly engageable with the cartridge to expel the substance (e.g., an anti-C5 antibody, or antigen-binding fragment thereof) therefrom, the driving assembly being operatively engaged with the activation button assembly and being configured to drive the cartridge from the sealed position to the unsealed position upon movement of the activation button assembly from the unactuated position to the actuated position. In some embodiments, the interior channel defines a length greater than a length of the cartridge. In some embodiments, the interference member comprises a cantilevered arm.

In some embodiments, the cantilevered arm defines a first end connected to the cartridge door and extends to a second, free end proximate the open end of the cartridge door. In some embodiments, the cartridge door comprises a sidewall, and the cantilevered arm defines a deflectable portion of the sidewall of the cartridge door. In some embodiments, the cantilevered arm is integrally formed with the sidewall of the cartridge door. In some embodiments, the sidewall of the cartridge door defines a first internal perimeter at the second, free end of the cantilevered arm when the cantilevered arm is in the resting position thereof, and the sidewall of the cartridge door defines a second internal perimeter at the second, free end of the cantilevered arm when the cantilevered arm is deflected from the resting position thereof, the first internal perimeter being smaller than a largest outer perimeter of the cartridge insertable into the interior channel, thereby limiting the insertion depth of the cartridge in the interior channel of the cartridge door, and the second internal perimeter being greater than the largest outer perimeter of the cartridge, thereby enabling advancement of the cartridge in the interior channel of the cartridge door.

In some embodiments, the activation button assembly comprises a post extending therefrom to a terminal end having a tapered face, the post having a post pathway along which the post travels from the unactuated position of the activation button assembly to the actuated position of the activation button assembly, and wherein the cantilevered arm comprises a tab laterally extending therefrom toward the post pathway, the tab having a hooked end facing the tapered face of the post, wherein movement of the activation button assembly from the unactuated position to the actuated position, in the closed position of the cartridge door, engages the post with the tab, the tapered face engaging and laterally translating the hooked end, and, in turn, deflecting the cantilevered arm away from the resting position thereof. In some embodiments, the activation button assembly is operatively connected to the injection needle, whereby movement of the activation button assembly from the unactuated position to the actuated position thereof drives the injection needle from the retracted position to the injection position thereof. In some embodiments, the cartridge door is pivotably mounted to the injector housing at an end of the cartridge door opposite the open end thereof.

In some embodiments, the injector (e.g., the OBI) comprises: an injector housing; an injection needle translatable between a retracted position, where at least a tip of the injection needle is contained within the injector housing, and an injection position, where at least the tip of the injection needle protrudes from the injector housing; an activation button assembly movably mounted to the injector housing and operatively connected to the injection needle, the activation button assembly being translatable from an unactuated position to an actuated position to drive the injection needle from the retracted position to the injection position; a cartridge door movably mounted to the injector housing between an open position and a closed position, the cartridge door comprising: an open end, an interior channel having a cartridge mounted therein, the cartridge containing a substance (e.g., an anti-C5 antibody, or antigen-binding fragment thereof) to be dispensed and having an opening at a front end of the cartridge and a flange at a rear end of the cartridge, and a cartridge piercing needle mounted within the interior channel and fluidly connected with the injection needle; and an interference member engaging the flange of the cartridge in a resting position of the interference member, thereby limiting an insertion depth of the cartridge in the interior channel of the cartridge door to a sealed position, wherein the cartridge piercing needle does not fully penetrate a pierceable septum in the sealed position; wherein: the cartridge door is movable to the closed position in the sealed position of the cartridge, and wherein movement of the activation button assembly from the unactuated position to the actuated position, when the cartridge door is in the closed position, deflects the interference member out of engagement with the flange of the cartridge, thereby enabling advancement of the cartridge in the interior channel of the cartridge door to an unsealed position, wherein the cartridge piercing needle fully penetrates the pierceable septum in the unsealed position to dispense the substance (e.g., an anti-C5 antibody, or antigen-binding fragment thereof) through the injection needle.

In some embodiments, the interference member comprises a cantilevered arm. In some embodiments, the cantilevered arm defines a first end connected to the cartridge door and extends to a second, free end proximate the open end of the cartridge door. In some embodiments, the cartridge door comprises a sidewall and the cantilevered arm defines a deflectable portion of the sidewall of the cartridge door. In some embodiments, the sidewall of the cartridge door defines a first internal perimeter at the second, free end of the cantilevered arm when the cantilevered arm is in the resting position thereof, and the sidewall of the cartridge door defines a second internal perimeter at the second, free end of the cantilevered arm when the cantilevered arm is deflected from the resting position thereof, the first internal perimeter being smaller than a perimeter of the rear end flange of the cartridge insertable into the interior channel, thereby limiting the insertion depth of the cartridge in the interior channel of the cartridge door, and the second internal perimeter being greater than the rear end flange perimeter of the cartridge, thereby enabling advancement of the cartridge in the interior channel of the cartridge door.

In some embodiments, the activation button assembly comprises a post extending therefrom to a terminal end having a tapered face, the post having a post pathway along which the post travels from the unactuated position of the activation button assembly to the actuated position of the activation button assembly, and wherein the cantilevered arm comprises a tab laterally extending therefrom toward the post pathway, the tab having a hooked end facing the tapered face of the post, wherein movement of the activation button assembly from the unactuated position to the actuated position, in the closed position of the cartridge door, engages the post with the tab, the tapered face engaging and laterally translating the hooked end, and, in turn, deflecting the cantilevered arm away from the resting position thereof.

In some embodiments, the injector (e.g., the OBI) is configured to receive a cartridge containing a substance (e.g., an anti-C5 antibody, or antigen-binding fragment thereof) to be dispensed, the cartridge having an opening sealed by a pierceable septum, the injector comprising: an injector housing; an activation button assembly movably mounted to the injector housing, the activation button assembly being translatable from an unactuated position to an actuated position; an injection needle; a cartridge door movably mounted to the injector housing between an open position and a closed position, the cartridge door comprising: an interior channel configured to receive the cartridge, and a cartridge piercing needle fluidly connected with the injection needle; and an interference member having a position configured to limit an insertion depth of the cartridge in the interior channel of the cartridge door to a sealed position, wherein the cartridge piercing needle does not fully penetrate the pierceable septum in the sealed position, wherein the cartridge door is movable to the closed position when the cartridge is in the sealed position, and wherein movement of the activation button assembly from the unactuated position to the actuated position, when the cartridge door is in the closed position, moves the interference member out of the position, thereby enabling advancement of the cartridge in the interior channel of the cartridge door to an unsealed position, wherein the cartridge piercing needle fully penetrates the pierceable septum in the unsealed position to inject the substance (e.g., an anti-C5 antibody, or antigen-binding fragment thereof) through the injection needle.

In some embodiments, the interior channel defines a length greater than a length of the cartridge. In some embodiments, the interference member comprises a cantilevered arm.

In some embodiments, the injector (e.g., the OBI) comprises: an injector housing; an injection needle translatable between a retracted position, at which at least a tip of the injection needle is contained within the injector housing, and an injection position, at which at least the tip of the injection needle protrudes from the injector housing; an activation button assembly movably mounted to the injector housing and operatively connected to the injection needle, the activation button assembly being translatable from an unactuated position to an actuated position to drive the injection needle from the retracted position to the injection position; a cartridge door movably mounted to the injector housing between an open position and a closed position, the cartridge door comprising: an open end, an interior channel having a cartridge mounted therein, the cartridge containing an antibody (e.g., an anti-C5 antibody, or antigen-binding fragment thereof) to be dispensed and having an opening at a front end of the cartridge sealed by a pierceable septum and a flange at a rear end thereof, and a cartridge piercing needle mounted within the interior channel and connected in fluid communication with the injection needle, the cartridge piercing needle being configured to fully penetrate the pierceable septum of the cartridge to connect the antibody (e.g., an anti-C5 antibody, or antigen-binding fragment thereof) within the cartridge in fluid communication with the injection needle; and a deflectable interference member engaging the rear end flange of the cartridge in a resting position of the interference member, thereby limiting an insertion depth of the cartridge into the interior channel of the cartridge door to a sealed position, at which the cartridge piercing needle does not fully penetrate the pierceable septum, wherein: the cartridge door is movable to the closed position in the sealed position of the cartridge, and movement of the activation button assembly from the unactuated position to the actuated position, in the closed position of the cartridge door, deflects the interference member out of engagement with the rear end flange of the cartridge, thereby enabling further advancement of the cartridge into the interior channel of the cartridge door to an unsealed position, at which the cartridge piercing needle fully penetrates the pierceable septum.

In some embodiments, the injector further comprises a driving assembly engageable with the cartridge to expel the antibody (e.g., an anti-C5 antibody, or antigen-binding fragment thereof) therefrom, the driving assembly being operatively engaged with the activation button assembly and being configured to drive the cartridge from the sealed position to the unsealed position upon movement of the activation button assembly from the unactuated position to the actuated position. In some embodiments, the deflectable interference member comprises a cantilevered arm. In some embodiments, the cantilevered arm defines a first end connected to the cartridge door and extending to a second, free end proximate the open end of the cartridge door. In some embodiments, the cartridge door comprises a sidewall and the cantilevered arm defines a deflectable portion of the sidewall of the cartridge door. In some embodiments, the sidewall of the cartridge door defines a first internal perimeter at the second, free end of the cantilevered arm when the cantilevered arm is in the resting position, and the sidewall of the cartridge door defines a second internal perimeter at the second, free end of the cantilevered arm when the cantilevered arm is deflected from the resting position, the first internal perimeter being smaller than a largest outer perimeter of the cartridge insertable into the interior channel, thereby limiting the insertion depth of the cartridge into the interior channel of the cartridge door, and the second internal perimeter being greater than the largest outer perimeter of the cartridge, thereby enabling further advancement of the cartridge into the interior channel of the cartridge door.

In some embodiments, the activation button assembly comprises a post extending therefrom to a terminal end having a tapered face, the post having a post pathway along which the post travels from the unactuated position of the activation button assembly to the actuated position of the activation button assembly, and wherein the cantilevered arm comprises a tab laterally extending therefrom toward the post pathway, the tab having a hooked end facing the tapered face of the post, wherein movement of the activation button assembly from the unactuated position to the actuated position, in the closed position of the cartridge door, engages the post with the tab, the tapered face engaging and laterally translating the hooked end, and, in turn, deflecting the cantilevered arm away from the resting position.

In some embodiments, the injector further comprises a biasing member connected with the activation button assembly and the injection needle, the biasing member being stabilized in a stored energy state in the unactuated position of the activation button assembly, and released in the actuated position of the activation button assembly into an energy releasing state to drive the injection needle from the retracted position to the injection position, wherein. a position of the activation button assembly between the unactuated position and the actuated position defines a threshold point, and movement of the activation button assembly beyond the threshold point secures the activation button assembly in the actuated position and the injection needle in the injection position. In some embodiments, the biasing member biases the activation button assembly into the unactuated position and returns the activation button assembly to the unactuated position in response to movement of the activation button assembly not surpassing the threshold point.

In some embodiments, the injector further comprises a needle hub movably mounted within the injector housing, the injection needle being supported by the needle hub, and the needle hub and the injection needle being translatable between the retracted position and the injection position. In some embodiments, the injector further comprises: an elongate first post connected with the injector housing and projecting upwardly therefrom, the elongate first post having an upper end comprising a downwardly inclined surface and an undercut underlying the inclined surface; and a deflectable second post connected with the injector housing and projecting upwardly therefrom, the deflectable second post including a flange supporting a portion of the needle hub thereon, thereby securing the needle hub and the injection needle in the retracted position, wherein: the activation button assembly comprises a downwardly projecting first arm having a flanged terminal end slidable along the inclined surface of the elongate first post, the first arm being elastically deflectable from an original state thereof, movement of the activation button assembly slides the flanged terminal end of the first arm along the inclined surface of the first post, thereby elastically deflecting the first arm away from the original state, the inclined surface and the undercut of the elongate first post meet at a vertex defining the threshold point, movement of the activation button assembly beyond the vertex triggers retraction of the first arm back toward the original state, thereby hooking the flanged terminal end onto the undercut of the elongate first post and securing the activation button assembly in the actuated position, and movement of the activation button assembly beyond the vertex also engages the flanged terminal end of the first arm with the second post and deflects the second post, whereby the deflected second post releases the needle hub, and, in turn, releases the biasing member into the energy releasing state to drive the needle hub and the injection needle from the retracted position to the injection position.

In some embodiments, provided herein is a method of dispensing an antibody (e.g., an anti-C5 antibody, or antigen-binding fragment thereof) from an injector (e.g., an OBI), the injector having an injector housing, an injection needle movable from a retracted position, at which at least a tip of the injection needle is contained within the injector housing, and an injection position, at which at least the tip of the injection needle protrudes from the injector housing, an activation button assembly movably mounted to the injector housing and operatively connected to the injection needle, a cartridge door movably mounted to the injector housing between an open position and a closed position, the cartridge door having an open end, an interior channel, and a cartridge piercing needle mounted within the interior channel and connected in fluid communication with the injection needle, and a driving assembly operatively engaged with the activation button assembly, the method comprising: inserting a cartridge into the interior channel of the cartridge door in the open position of the cartridge door, the cartridge containing the antibody (e.g., an anti-C5 antibody, or antigen-binding fragment thereof) in a sealed reservoir thereof and having an opening at a front end of the reservoir sealed by a pierceable septum and a flange at a rear end of the reservoir; engaging the rear end flange of the cartridge with a deflectable interference member of the injector in a resting position of the interference member, thereby limiting an insertion depth of the cartridge into the interior channel of the cartridge door to a sealed position, at which the cartridge piercing needle does not fully penetrate the pierceable septum; moving the cartridge door into the closed position; and moving the activation button assembly from an unactuated position to an actuated position thereby: deflecting the interference member out of engagement with the rear end flange of the cartridge, activating the driving assembly to advance the rear end flange of the cartridge past the interference member and drive the cartridge from the sealed position to an unsealed position, at which the cartridge piercing needle fully penetrates the pierceable septum of the cartridge and connects the antibody (e.g., an anti-C5 antibody, or antigen-binding fragment thereof) within the cartridge in fluid communication with the injection needle, and driving the injection needle from the retracted position to the injection position and dispensing the antibody (e.g., an anti-C5 antibody, or antigen-binding fragment thereof) therefrom.

In some embodiments, the driving of the injection needle to the injection position comprises protruding at least the tip of the injection needle from the injector housing, and dispensing the antibody (e.g., an anti-C5 antibody, or antigen-binding fragment thereof) from the injection needle. In some embodiments, the injector further includes a sensor connected to a control assembly, the method further comprising: detecting, via the sensor, at least one of (i) movement of the activation button assembly from the unactuated position thereof to the actuated position or (ii) movement of the injection needle from the retracted position thereof into the injection position; and activating the driving assembly, via the control assembly, upon said detecting.

In some embodiments, the activation button assembly includes a post extending therefrom to a terminal end having a tapered face, the post having a post pathway along which the post travels from the unactuated position of the activation button assembly to the actuated position of the activation button assembly, and wherein the interference member includes a cantilevered arm defining a deflectable portion of a sidewall of the cartridge door, whereby a first end of the cantilevered arm is connected to the sidewall and extends to a second, free end proximate the open end of the cartridge door, the cantilevered arm including a tab laterally extending therefrom toward the post pathway, the tab having a hooked end facing the tapered face of the post, and wherein moving of the activation button assembly from the unactuated position to the actuated position comprises engaging the tapered face of the post with the hooked end of the tab, laterally translating the hooked end, and, in turn, deflecting the cantilevered arm out of engagement with the rear end flange of the cartridge.

In some embodiments, the injector (e.g., the OBI) comprises: an injector housing; an injection needle translatable between a retracted position, at which at least a tip of the injection needle is contained within the injector housing, and an injection position, at which at least the tip of the injection needle protrudes from the injector housing; a cartridge door movably mounted to the injector housing between an open position and a closed position, the cartridge door comprising: an open end, an interior channel having a cartridge mounted therein, the cartridge containing an antibody (e.g., an anti-C5 antibody, or antigen-binding fragment thereof) to be dispensed and having an opening at a front end of the cartridge sealed by a pierceable septum and a flange at a rear end thereof, and a cartridge piercing needle mounted within the interior channel and connected in fluid communication with the injection needle, the cartridge piercing needle being configured to fully penetrate the pierceable septum of the cartridge to connect the antibody (e.g., an anti-C5 antibody, or antigen-binding fragment thereof) within the cartridge in fluid communication with the injection needle; and a deflectable interference member engaging the rear end flange of the cartridge in a resting position of the interference member, thereby limiting an insertion depth of the cartridge into the interior channel of the cartridge door to a sealed position, at which the cartridge piercing needle does not fully penetrate the pierceable septum.

In some embodiments, the injector further comprises an activation button assembly movably mounted to the injector housing and operatively connected to the injection needle, the activation button assembly being translatable from an unactuated position to an actuated position to drive the injection needle from the retracted position to the injection position. In some embodiments, the injector further comprises a driving assembly engageable with the cartridge to expel the antibody (e.g., an anti-C5 antibody, or antigen-binding fragment thereof) therefrom, the driving assembly being operatively engaged with the activation button assembly and being configured to drive the cartridge upon movement of the activation button assembly from the unactuated position to the actuated position.

In some embodiments, the deflectable interference member comprises a cantilevered arm defining a first end connected to the cartridge door and extending to a second, free end proximate the open end of the cartridge door. In some embodiments, the cartridge door comprises a sidewall and the cantilevered arm defines a deflectable portion of the sidewall of the cartridge door. In some embodiments, the sidewall of the cartridge door defines a first internal perimeter at the second, free end of the cantilevered arm when the cantilevered arm is in the resting position, and the sidewall of the cartridge door defines a second internal perimeter at the second, free end of the cantilevered arm when the cantilevered arm is deflected from the resting position, the first internal perimeter being smaller than a largest outer perimeter of the cartridge insertable into the interior channel, thereby limiting the insertion depth of the cartridge into the interior channel of the cartridge door, and the second internal perimeter being greater than the largest outer perimeter of the cartridge, thereby enabling further advancement of the cartridge into the interior channel of the cartridge door.

In some embodiments, the activation button assembly comprises a post extending therefrom to a terminal end having a tapered face, the post having a post pathway along which the post travels from the unactuated position of the activation button assembly to the actuated position of the activation button assembly, and wherein the cantilevered arm comprises a tab laterally extending therefrom toward the post pathway, the tab having a hooked end facing the tapered face of the post, wherein movement of the activation button assembly from the unactuated position to the actuated position, in the closed position of the cartridge door, engages the post with the tab, the tapered face engaging and laterally translating the hooked end, and, in turn, deflecting the cantilevered arm away from the resting position.

In some embodiments, the injector further comprises a biasing member connected with the activation button assembly and the injection needle, the biasing member being stabilized in a stored energy state in the unactuated position of the activation button assembly, and released in the actuated position of the activation button assembly into an energy releasing state to drive the injection needle from the retracted position to the injection position, wherein: a position of the activation button assembly between the unactuated position and the actuated position defines a threshold point, and movement of the activation button assembly beyond the threshold point secures the activation button assembly in the actuated position and the injection needle in the injection position. In some embodiments, the biasing member biases the activation button assembly into the unactuated position and returns the activation button assembly to the unactuated position in response to movement of the activation button assembly not surpassing the threshold point.

In some embodiments, the injector further comprises a needle hub movably mounted within the injector housing, the injection needle being supported by the needle hub, and the needle hub and the injection needle being translatable between the retracted position and the injection position.

In some embodiments, the injector further comprises: an elongate first post connected with the injector housing and projecting upwardly therefrom, the elongate first post having an upper end comprising a downwardly inclined surface and an undercut underlying the inclined surface, and a deflectable second post connected with the injector housing and projecting upwardly therefrom, the deflectable second post including a flange supporting a portion of the needle hub thereon, thereby securing the needle hub and the injection needle in the retracted position, wherein: the activation button assembly comprises a downwardly projecting first arm having a flanged terminal end slidable along the inclined surface of the elongate first post, the first arm being elastically deflectable from an original state thereof, movement of the activation button assembly slides the flanged terminal end of the first arm along the inclined surface of the first post, thereby elastically deflecting the first arm away from the original state, the inclined surface and the undercut of the elongate first post meet at a vertex defining the threshold point, movement of the activation button assembly beyond the vertex triggers retraction of the first arm back toward the original state, thereby hooking the flanged terminal end onto the undercut of the elongate first post and securing the activation button assembly in the actuated position, and movement of the activation button assembly beyond the vertex also engages the flanged terminal end of the first arm with the second post and deflects the second post, whereby the deflected second post releases the needle hub, and, in turn releases the biasing member into the energy releasing state to drive the needle hub and the injection needle from the retracted position to the injection position.

In some embodiments, the cartridge has a volume of 10 mL. In some embodiments, the OBDS is configured for self-administration of the anti-C5 antibody, or antigen-binding fragment thereof, subcutaneously to the patient.

The OBDS may be configured for subcutaneous administration of the anti-C5 antibody, or antigen-binding fragment thereof, to any suitable site. In some embodiments, the OBDS is configured for subcutaneous administration of the anti-C5 antibody, or antigen-binding fragment thereof, to a site selected from the patient's arm, abdomen, and thigh.

Additionally, provided herein is a needle syringe configured for administration of an effective amount of an anti-C5 antibody, or antigen-binding fragment thereof, to treat aHUS or PNH in a pediatric human patient in need thereof, wherein the patient is ≥10 to <20 kg of body weight and is complement inhibitor treatment-naïve, eculizumab-experienced, or ravulizumab-experienced, wherein the anti-C5 antibody, or antigen-binding fragment thereof, is administered subcutaneously once per week at a dose of 150 mg.

Also provided herein is a needle syringe configured for treating aHUS or PNH in a pediatric human patient in need thereof, wherein the needle syringe is configured for subcutaneous administration and comprises 150 mg of an anti-C5 antibody, or antigen-binding fragment thereof, wherein the patient is ≥10 to <20 kg of body weight and is complement inhibitor treatment-naïve, eculizumab-experienced, or ravulizumab-experienced.

In some embodiments, the 150 mg dose of the anti-C5 antibody, or antigen-binding fragment thereof, is formulated at pH 7.4 and a concentration of 100 mg/mL in sodium phosphate, sucrose, L-arginine, PS80, and water for injection. In some embodiments, the needle syringe is configured for administration of the anti-C5 antibody, or antigen-binding fragment thereof, to a site selected from the patient's arm, abdomen, and thigh. In some embodiments, the patient is less than 21 years of age (e.g., 20, 19, 18, 17, 16, 15, 10, 5, or 2 years of age). In some embodiments, the patient is between 2 and 18 years of age (e.g., 2, 3, 4, 5, 6, 7, 8, 9, 10, 11, 12, 13, 14, 15, 16, 17, or 18 years of age).

Any suitable anti-C5 antibody, or antigen-binding fragment thereof, may be included in an OBDS or needle syringe as provided herein, including any suitable anti-C5 antibody or antigen-binding fragment thereof disclosed herein. In some embodiments, the anti-C5 antibody, or antigen-binding fragment thereof, comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6. In some embodiments, the anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering. In some embodiments, the anti-C5 antibody, or antigen-binding fragment thereof, comprises a heavy chain variable region comprising the amino acid sequence of SEQ ID NO: 12 and a light chain variable region comprising the amino acid sequence of SEQ ID NO: 8. In some embodiments, the heavy chain variable region comprises an N-terminal pyroglutamate residue. In some embodiments, the anti-C5 antibody, or antigen-binding fragment thereof, further comprises a heavy chain constant region comprising the amino acid sequence of SEQ ID NO: 13. In some embodiments, the anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.

### Anti-C5 Antibodies

C5 complement, which forms part of the later stages of the complement pathway, is cleaved into C5a and C5b. C5a is a potent anaphylatoxin, chemotactic factor, and cell-activating molecule that mediates multiple pro-inflammatory and pro-thrombotic activities (Matis L A, et al., Nat. Med. 1995; 1:839-42; Prodinger et al., Complement. In: Paul W E, editor. Fundamental immunology (4th ed). Philadelphia: Lippincott-Raven Publishers; 1999. p. 967-95). C5b recruits the terminal complement components C6, C7, C8, and C9 to form the pro-inflammatory, pro-thrombotic cytolytic pore molecule C5b-9, a process that under normal circumstances would be blocked on the red blood cell (RBC) membrane by CD59. In patients with PNH, however, these final steps proceed unchecked, culminating in hemolysis and the release of free hemoglobin, as well as platelet activation (Hill, et al., Blood 2013; 121:4985-96). The signs and symptoms of PNH can be attributed to chronic, uncontrolled complement C5 cleavage, and release of C5a and C5b-9 leading to RBC hemolysis, which together result in (Hill, et al., Blood 2013; 121:4985-96; Brodsky R A., Blood. 2014; 124:2804-1):
(1) Release of intracellular free hemoglobin and lactate dehydrogenase (LDH) into circulation as a direct consequence of hemolysis,
(2) Irreversible binding to and inactivation of nitric oxide (NO) by hemoglobin, and inhibition of NO synthesis,
(3) Vasoconstriction and tissue-bed ischemia due to absence of vasodilatory NO, as well as possible microthrombi manifesting as abdominal pain and dysphagia,
(4) Platelet activation, and
(5) A pro-inflammatory and prothrombotic state.

The anti-C5 antibodies described herein bind to complement component C5 (e.g., human complement C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes. Anti-C5 antibodies (or VH/VL domains or other antigen binding fragments derived therefrom) suitable for use herein can be generated using methods known in the art. Art-recognized anti-C5 antibodies can also be used. Antibodies that compete with any of these art-recognized antibodies for binding to C5 also can also be used.

Eculizumab (also known as Soliris®) is an anti-C5 antibody comprising heavy and light chains having sequences shown in SEQ ID NO: 10 and 11, respectively, or antigen binding fragments and variants thereof. Eculizumab is described in PCT/US2007/006606 and U.S. Pat. No. 9,732,149, the teachings of which are hereby incorporated by reference. In one embodiment the anti-C5 antibody, comprises the CDR1, CDR2 and CDR3 domains of the VH region of eculizumab having the sequence set forth in SEQ ID NO: 7, and the CDR1, CDR2 and CDR3 domains of the VL region of eculizumab having the sequence set forth in SEQ ID NO: 8. In another embodiment, the antibody comprises heavy chain CDR1, CDR2 and CDR3 domains having the sequences set forth in SEQ ID NOs: 1, 2 and 3, respectively, and light chain CDR1, CDR2 and CDR3 domains having the sequences set forth in SEQ ID NOs: 4, 5 and 6, respectively. In another embodiment, the antibody comprises VH and VL regions having the amino acid sequences set forth in SEQ ID NO: 7 and SEQ ID NO: 8, respectively.

Ravulizumab (also known as BNJ441, ALXN1210, ULTOMIRIS®, or ravulizumab-cwvz) is an anti-C5 antibody comprising heavy and light chains having the sequences shown in SEQ ID NOs:14 and 11, respectively, or antigen binding fragments and variants thereof. Ravulizumab is described in PCT/US2015/019225 and U.S. Pat. No. 9,079,949, the teachings of which are hereby incorporated by reference in their entirety. Ravulizumab is a humanized monoclonal antibody that is structurally related to eculizumab (Soliris®). Ravulizumab was derived through minimal targeted engineering of eculizumab by introducing four unique amino acid substitutions into the heavy chain, with the objective of enhancing the duration of terminal complement inhibition, while maintaining key eculizumab attributes. Ravulizumab and eculizumab share over 99% primary sequence identity and have very similar pharmacology. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In some embodiments, the anti-C5 antibody, or antigen-binding fragment thereof, includes CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively. In some embodiments, the anti-C5 antibody, or antigen-binding fragment thereof, includes a variant human Fc constant region that binds to human neonatal Fc receptor (FcRn), wherein the variant human Fc CH3 constant region includes Met-429-Leu and Asn-435-Ser substitutions at residues corresponding to methionine 428 and asparagine 434 of a native human IgG Fc constant region, each in EU numbering. In some embodiments, the anti-C5 antibody, or antigen-binding fragment thereof, includes a heavy chain variable region including the amino acid sequence of SEQ ID NO: 12 and a light chain variable region including the amino acid sequence of SEQ ID NO: 8. In some embodiments, the anti-C5 antibody, or antigen-binding fragment thereof, further includes a heavy chain constant region including in the amino acid sequence of SEQ ID NO: 13. In some embodiments, the anti-C5 antibody, or antigen-binding fragment thereof, includes a heavy chain polypeptide including the amino acid sequence of SEQ ID NO: 14 and a light chain polypeptide including the amino acid sequence of SEQ ID NO: 11. In some embodiments, the anti-C5 antibody, or antigen-binding fragment thereof, includes a heavy chain polypeptide having the amino acid sequence of SEQ ID NO: 14 except for lacking the C-terminal lysine residue and a light chain polypeptide including the amino acid sequence of SEQ ID NO: 11. In some embodiments, the heavy chain amino acid sequence includes a N-terminal pyroglutamate residue.

In some embodiments, the anti-C5 antibody is ravulizumab.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs” (Thomas, T. et al., Mol. Immunol., 33:1389-401, 1996).

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody or antigen-binding fragment thereof comprises heavy chain CDR1, CDR2 and CDR3 domains having the sequences set forth in SEQ ID NOs: 21, 22 and 23, respectively, and light chain CDR1, CDR2 and CDR3 domains having the sequences set forth in SEQ ID NOs: 24, 25 and 26, respectively. In another embodiment, the antibody or antigen-binding fragment thereof comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody or antigen-binding fragment thereof comprises heavy chain CDR1, CDR2 and CDR3 domains having the sequences set forth in SEQ ID NOs: 29, 30 and 31, respectively, and light chain CDR1, CDR2 and CDR3 domains having the sequences set forth in SEQ ID NOs: 32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36.

Another exemplary anti-C5 antibody is the 305LO5 antibody described in U.S. Pat. No. 9,765,135. In one embodiment, the antibody comprises the heavy and light chain CDRs or variable regions of the 305LO5 antibody. In another embodiment, the antibody or antigen-binding fragment thereof comprises heavy chain CDR1, CDR2 and CDR3 domains having the sequences set forth in SEQ ID NOs: 37, 38 and 39, respectively, and light chain CDR1, CDR2 and CDR3 domains having the sequences set forth in SEQ ID NOs: 40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:44.

Another exemplary anti-C5 antibody is the SKY59 antibody (Fukuzawa T. et al., Sci. Rep., 7:1080, 2017). In one embodiment, the antibody comprises the heavy and light chain CDRs or variable regions of the SKY59 antibody. In another embodiment, the antibody or antigen-binding fragment thereof comprises a heavy chain comprising SEQ ID NO:45 and a light chain comprising SEQ ID NO:46.

Another exemplary anti-C5 antibody is the H4H12166PP antibody described in PCT/US2017/037226 and U.S. Pat. No. 10,633,434. In one embodiment, the antibody comprises the heavy and light chain CDRs or variable regions of the H4H12166PP antibody. In another embodiment, the antibody or antigen-binding fragment thereof comprises the VH region of the H4H12166PP antibody having the sequence set forth in SEQ ID NO:47, and the VL region of the H4H12166PP antibody having the sequence set forth in SEQ ID NO:48. In another embodiment, the antibody or antigen-binding fragment thereof comprises a heavy chain comprising SEQ ID NO:49 and a light chain comprising SEQ ID NO:50.

In some embodiments, the patient has previously been treated with eculizumab or a biosimilar thereof or an anti-C5 antibody selected from tesidolumab, crovalimab, CAN106, or pozelimab. In some embodiments, the biosimilar of eculizumab comprises ABP 959, ELIZARIA®, SB12, ISU305, ABLYZE®, or BCD 148. In some embodiments, the administration cycle starts about two weeks after the patient's last dose of eculizumab. In some embodiments, the patient has been treated with eculizumab for at least 90 days prior to Day 1 of the administration cycle. In some embodiments, (i) the patient weighs <20 kg, and the administration cycle starts about 4 weeks after the patient's last dose of ravulizumab or (ii) the patient weighs ≥20 kg, and the administration cycle starts about 8 weeks after the patient's last dose of ravulizumab.

In some embodiments, an anti-C5 antibody described herein comprises a heavy chain CDR1 comprising or consisting of the following amino acid sequence: GHIFSNYWIQ (SEQ ID NO:19). In some embodiments, an anti-C5 antibody described herein comprises a heavy chain CDR2 comprising or consisting of the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQG
LEWMGEILPGSGHTEYTENFKDRVTMTRDTSTSTVYMELSSLRS
EDTAVYYCARYFFGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAP
KLLIYGATNLADGVPSRFSGSGSGTDFTLTISSLQPEDFATYYC
QNVLNTPLTFGQGTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can comprise, for example, one or more (e.g., two, three, four, five, six, seven or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples (see, e.g., PCT/US2015/019225 and U.S. Pat. No. 9,079,949 the disclosures of each of which are incorporated herein by reference in their entirety).

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) M428L or T250Q/M428L substitutions (Hinton, P. et al., J. Biol. Chem., 279:6213-6, 2004; Hinton, P. et al., J. Immunol., 176:346-56, 2006); and (3) N434A or T307/E380A/N434A substitutions (Petkova, S. et al., Int. Immunol., 18:1759-69, 2006). Additional substitution pairings, e.g., P2571/Q3111, P2571/N434H, and D376V/N434H (Datta-Mannan, A. et al., J. Biol. Chem., 282:1709-17, 2007) are also contemplated herein.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions or deletions relative to the native constant region from which it was derived. In some embodiments, the variant Fc constant region comprises one or more amino acid substitutions selected from the group consisting of: M252Y, S254T, T256E, N434S, M428L, V2591, T2501 and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. The 428L/434S double substitution when used in a IgG2/4 chimeric Fc, for example, may correspond to 429L and 435S as in the M429L and N435S variants found in BNJ441 (ravulizumab) and described in U.S. Pat. No. 9,079,949, the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256, alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307, alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380, alanine for glutamic acid at position 382, alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein can comprise a heavy chain polypeptide comprising the amino acid sequence of SEQ ID NO:14 and/or a light chain polypeptide comprising the amino acid sequence of SEQ ID NO:11. Alternatively, the anti-C5 antibodies for use in the methods described herein can comprise a heavy chain polypeptide comprising the amino acid sequence of SEQ ID NO:20 and/or a light chain polypeptide comprising the amino acid sequence of SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975) nM. In some embodiments, the K_{D} of the anti-C5 antibody or antigen-binding fragment thereof is no greater than 1 (e.g., no greater than 0.9, 0.8, 0.7, 0.6, 0.5, 0.4, 0.3 or 0.2) nM.

In some embodiments, the [(K_{D} of the antibody for C5 at pH 6.0 at 25° C.)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180,190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA) (see, e.g., Benny K. C. Lo (2004) “Antibody Engineering: Methods and Protocols,” Humana Press (ISBN: 1588290921); Johne, B. et al., J. Immunol. Meth., 160:191-8, 1993; Jonsson, U. et al., Ann. Biol. Clin., 51:19-26, 1993; Jonsson, U. et al., Biotechniques, 11:620-7, 1991). Additional methods for measuring, for example, affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=k_{d}/kₐ. Such determinations preferably are measured at 25° C. or 37° C. The kinetics of antibody binding to human C5 can be determined, for example, at pH 8.0, 7.4, 7.0, 6.5 and 6.0 via surface plasmon resonance (SPR) on a BIAcore 3000 instrument using an anti-Fc capture method to immobilize the antibody. In one embodiment, the anti-C5 antibody, or antigen-binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay described by Kabat and Mayer (eds.), “Experimental Immunochemistry, 2^{nd} Edition,” 135-240, Springfield, IL, CC Thomas (1961), pages 135-139, or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using these or other suitable assays, candidate agents capable of inhibiting human complement component C5 can be screened.

Immunological techniques such as, but not limited to, ELISA can be used to measure the protein concentration of C5 and/or its split products to determine the ability of an anti-C5 antibody or antigen-binding fragment thereof to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody or antigen-binding fragment thereof on complement activation. To determine the effect of an anti-C5 antibody or antigen-binding fragment thereof on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody or antigen-binding fragment thereof in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody or antigen-binding fragment thereof. In some embodiments, the test serum is a C5-deficient serum reconstituted with a C5 polypeptide.

To determine the effect of an anti-C5 antibody, or antigen-binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody or antigen-binding fragment thereof in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody or antigen-binding fragment thereof.

In some embodiments, C5 activity, or inhibition thereof, is quantified using a CH50eq assay. The CH50eq assay is a method for measuring the total classical complement activity in serum. This test is a Iytic assay that uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured. Briefly, to activate the classical complement pathway, undiluted serum samples (e.g., reconstituted human serum samples) are added to microassay wells containing the antibody-sensitized erythrocytes to thereby generate TCC. Next, the activated serum samples are diluted in microassay wells, which are coated with a capture reagent (e.g., an antibody that binds to one or more components of the TCC). The TCC present in the activated samples bind to the monoclonal antibodies coating the surface of the microassay wells The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90 or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50eq assay.

In some embodiments, an anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody or antigen-binding fragment thereof described herein has a serum half-life that is at least 20 (e.g., at least 30, 35, 40, 45, 50, 55, 60, 65, 70, 75, 80, 85, 90, 95, 100, 125, 150, 175, 200, 250, 300, 400, 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the “same epitope on CS” with the antibodies described herein include, for example, epitope mapping methods, such as, x-ray analyses of crystals of antigen:antibody complexes that provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. Computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In some embodiments, an antibody competes with and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies can bind, for example, to the same epitope, an overlapping epitope or to adjacent epitopes (e.g., as evidenced by steric hindrance).

Anti-C5 antibodies or antigen-binding fragments thereof described herein, used in the methods described herein, can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (Köhler, G. & Milstein, C., Eur. J. Immunol., 6:511-9, 1976). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. One can alternatively isolate DNA sequences that encode a monoclonal antibody or a binding fragment thereof by screening a DNA library from human B cells (Huse, W. et al., Science, 246:1275-81, 1989).

### Compositions

Provided herein are compositions comprising an anti-C5 antibody, or antigen-binding fragment thereof. In one embodiment, the composition comprises an anti-C5 antibody comprising the CDR1, CDR2 and CDR3 domains in a heavy chain variable region having the sequence set forth in SEQ ID NO: 12, and the CDR1, CDR2 and CDR3 domains in a light chain variable region having the sequence set forth in SEQ ID NO: 8. In another embodiment, the anti-C5 antibody comprises heavy and light chains having the sequences shown in SEQ ID NOs: 14 and 11, respectively. In another embodiment, the anti-C5 antibody comprises heavy and light chains having the sequences shown in SEQ ID NOs: 20 and 11, respectively.

For example, pharmaceutical compositions comprising ravulizumab, either alone or in combination with prophylactic agents, therapeutic agents, and/or pharmaceutically acceptable carriers are provided. The pharmaceutical compositions comprising ravulizumab provided herein are for use in, for example, diagnosing, detecting or monitoring a disorder, in preventing, treating, managing or ameliorating a disorder or one or more symptoms thereof, and/or in research. Formulations of pharmaceutical compositions, either alone or in combination with prophylactic agents, therapeutic agents, and/or pharmaceutically acceptable carriers, are known in the art.

The compositions can be formulated as a pharmaceutical solution, e.g., for administration to a subject for the treatment or prevention of a complement-associated disorder including PNH or aHUS.

The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The composition can be formulated according to known methods (Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN. 091733096X)). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1/z years or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery can, for example, be in the form of injectable or infusible solutions. The compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion. In one embodiment, the antibodies are formulated for intravenous administration. In particular embodiments, the antibodies are formulated for subcutaneous administration.

An exemplary, non-limiting range for a loading dose (e.g., for a pediatric human patient who is complement inhibitor naïve or eculizumab-experienced) of a therapeutically or prophylactically effective amount of an anti-C5 antibody, or antigen-binding fragment thereof, provided herein (e.g., ravulizumab) is:
(i) 600 mg to a patient weighing ≥10 to <20 kg,
(ii) 900 mg to a patient weighing ≥20 to <30 kg,
(iii) 1200 mg to a patient weighing ≥30 to <40 kg,
(iv) 2400 mg to a patient weighing ≥40 to <60 kg, and
(v) 2700 mg to a patient weighing ≥60 kg.

An exemplary, non-limiting range for a maintenance dose (e.g., for a pediatric human patient who is complement inhibitor naïve or eculizumab-experienced) of a therapeutically or prophylactically effective amount of an anti-C5 antibody, or antigen-binding fragment thereof, provided herein (e.g., ravulizumab) is:
(i) 150 mg to a patient weighing ≥10 to <20 kg,
(ii) 245 mg to a patient weighing ≥20 to <40 kg, and
(iii) 490 mg to a patient weighing ≥40 kg.

An exemplary, non-limiting range for a dose (e.g., for a pediatric human patient who is ravulizumab-experienced) of a therapeutically or prophylactically effective amount of an anti-C5 antibody, or antigen-binding fragment thereof, provided herein (e.g., ravulizumab) is:
(i) 150 mg to a patient weighing ≥10 to <20 kg,
(ii) 245 mg to a patient weighing ≥20 to <40 kg, and
(iii) 490 mg to a patient weighing ≥40 kg.

It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed methods.

### Combination Therapy

An anti-C5 antibody provided herein also can be administered with one or more additional medicaments or therapeutic agents useful in the treatment of PNH or aHUS. The additional agent can be, for example, a therapeutic agent art-recognized as being useful to treat PNH or aHUS. The combination can also include more than one additional agents, e.g., two or three additional agents. The subcutaneous formulation containing the anti-C5 antibody of the disclosure can be administered with an agent that is a protein, a peptide, a carbohydrate, a drug, a small molecule, or a genetic material (e.g., DNA or RNA). In various embodiments, the agent is one or more cholinesterase inhibitors, one or more corticosteroids, and/or one or more immunosuppressive drugs (most commonly azathioprine (AZA), cyclosporin, and/or mycophenolate mofetil (MMF)).

### Outcomes

Provided herein are systems, devices, and methods for treating PNH in a pediatric patient comprising administering to the patient an anti-C5 antibody, or antigen-binding fragment thereof. Symptoms of PNH, especially in pediatric patients, are described in WO2019231983 and WO2022011086, including, the clinical trial identified as ALXN1210-PNH-304 (NCT03406507); pediatric PNH study), the disclosure(s) in which are incorporated by reference herein. They include, but are not limited to, fatigue (e.g., tiredness, difficulty performing daily activities, trouble concentrating, dizziness, weakness), pain (e.g., stomach pain, leg pain or swelling, chest pain, back pain), dark-colored urine, shortness of breath, difficulty swallowing, yellowing of the skin and/or eyes, erectile dysfunction, blood clots, kidney disease, damage to organs, stroke, or heart attack. Patients treated according to the methods disclosed herein preferably experience improvement in at least one sign of PNH. For example, the treatment may produce at least one therapeutic effect selected from the group consisting of a reduction or cessation in fatigue, abdominal pain, dyspnea, dysphagia, chest pain, and erectile dysfunction.

Also provided herein are methods for treating aHUS in a patient comprising administering to the patient an anti-C5 antibody, or antigen-binding fragment thereof. Symptoms of aHUS include, but are not limited to, severe hypertension, proteinuria, uremia, lethargy/fatigue, irritability, thrombocytopenia, microangiopathic hemolytic anemia, and renal function impairment (e.g., acute renal failure). Patients treated according to the methods disclosed herein preferably experience improvement in at least one sign of aHUS. For example, the treatment may produce at least one therapeutic effect selected from the group consisting of a reduction or cessation in severe hypertension, proteinuria, uremia, lethargy/fatigue, irritability, thrombocytopenia, microangiopathic hemolytic anemia, and renal function impairment (e.g., acute renal failure).

In another embodiment, improvement is measured by terminal complement inhibition.

In other embodiments, the treatment produces a shift toward normal levels of a hemolysis-related hematologic biomarker selected from the group consisting of free hemoglobin, haptoglobin, reticulocyte count, PNH red blood cell (RBC) clone and D-dimer. In some embodiments, the treatment results in achievement of stabilized hemoglobin. In some embodiments, the treatment results in achievement of stabilized hemoglobin through Week 10 and Week 52.

In some embodiments, the treatment results in a change from baseline in PNH red blood cell (RBC) clone size. In some embodiments, the treatment results in a change from baseline in PNH RBC clone size at Week 52.

In other embodiments, the treatment results in achievement of transfusion avoidance. In some embodiments, the treatment results in achievement of transfusion avoidance through Week 10 and Week 52.

In other embodiments, the treatment results in a reduction in the incidence of breakthrough hemolysis relative to baseline. In some embodiments, the treatment results in a reduction in the incidence of breakthrough hemolysis through Week 10 and Week 52. In another embodiment, the treatment results in an elimination of breakthrough hemolysis during the treatment period.

In other embodiments, the treatment produces a reduction in major adverse vascular events (MAVEs).

In other embodiments, the treatment produces a shift toward normal levels of a chronic disease associated biomarker selected from the group consisting estimated glomerular filtration rate (eGFR) and spot urine:albumin:creatinine and plasma brain natriuretic peptide (BNP). In some embodiments, the treatment results in a change in observed value and change from baseline in eGFR. In some embodiments, the treatment results in a change in observed value and change from baseline in eGFR through Week 10 and Week 52.

In some embodiments, the treatment in results in a change in dialysis requirement status relative to baseline. In some embodiments, the treatment in results in a change in dialysis requirement status relative to baseline through Week 10 and Week 52.

In some embodiments, the treatment results in a change in observed value and a change from baseline in serum creatinine. In some embodiments, the treatment results in a change in observed value and a change from baseline in serum creatinine through Week 10 and Week 52.

In some embodiments, the treatment results in a change in observed value and change from baseline in hematologic parameters selected from platelets, LDH, and/or hemoglobin. In some embodiments, the treatment results in a change in observed value and change from baseline in hematologic parameters selected from platelets, LDH, and/or hemoglobin through Week 10 and Week 52. In some embodiments, the treatment results in a reduction of hemolysis as assessed by LDH levels.

In some embodiments, the patient is not administered an immunosuppressive therapy comprising a steroid, a mammalian target of rapamycin (mTOR) inhibitor, or a calcineurin inhibitor unless it is as part of an established post-transplant anti-rejection regimen, the patient has confirmed anti-complement factor antibodies requiring immunosuppressive therapy, the steroid is being used for treatment of a condition other than aHUS, or the steroid was initiated empirically prior to treatment and is being tapered as standard of care. In some embodiments, the mTOR inhibitor may be sirolimus or everolimus. In some embodiments, the calcineurin inhibitor may be cyclosporine or tacrolimus.

In some embodiments of any of the foregoing aspects, the treatment maintains a serum trough concentration of <0.5 μg/mL of free C5 during the administration cycle and/or a serum concentration of 175 μg/mL of the anti-C5 antibody, or antigen-binding fragment thereof during the administration cycle. In some embodiments, the method further includes monitoring the concentration of: serum anti-C5 antibody, or antigen-binding fragment thereof, and/or serum free C5. In some embodiments, the concentration of serum anti-C5 antibody, or antigen-binding fragment thereof, and/or serum free C5 is determined from blood samples collected before and after each administration of the anti-C5 antibody, or antigen-binding fragment thereof. In some embodiments, the blood samples are collected within 30 minutes prior to the administration and within 60 minutes after each administration of the anti-C5 antibody, or antigen-binding fragment thereof.

In other embodiments, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale. In another embodiment, the treatment produces a change from baseline in quality of life, assessed via the Functional Assessment of Chronic Illness Therapy (FACIT)-Fatigue Scale, version 4 and the European Organisation for Research and Treatment of Cancer, Quality of Life Questionnaire-Core 30 Scale by at least 7 points from the patients untreated baseline score. In some embodiments, the treatment produces a change from baseline in patient-reported fatigue, as measured by Pediatric FACIT-Fatigue, optionally wherein the patient is ≥8 years of age. In some embodiments, the treatment produces a change from baseline in PedsQL 4.0 Generic Core Scale.

In another embodiment, the treatment results in no change in quality of life (QoL) as assessed via the Functional Assessment of Chronic Illness Therapy (FACIT)-Fatigue Scale, Version 4, from baseline to through Week 10 and Week 52. In another embodiment, the treatment results in an increase in quality of life (QoL) assessed via the Functional Assessment of Chronic Illness Therapy (FACIT)-Fatigue Scale, Version 4, from baseline through Week 10 and Week 52.

In other embodiments, lactate dehydrogenase (LDH) levels are used to evaluate responsiveness to a therapy (e.g., a reduction of hemolysis as assessed by lactate dehydrogenase (LDH) levels is indicative of an improvement in at least one sign of PNH). LDH is a marker of intravascular hemolysis (Hill, A. et al., Br. J. Haematol., 149:414-25, 2010; Hillmen, P. et al., N. Engl. J. Med., 350:552-9, 2004; Parker, C. et al., Blood, 106:3699-709, 2005). Red blood cells contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y et al., Int. J. Hematol., 93:36-46, 2011). LDH concentration obtained at baseline and then serially throughout a treatment period, is an important measure of hemolysis. Baseline levels of cell-free plasma hemoglobin are highly elevated in patients with PNH with LDH ≥1.5-fold above the upper limit of normal (LDH_1.5×ULN), with a significant correlation between LDH and cell-free plasma hemoglobin (Hillmen, P. et al., N. Engl. J. Med., 355:1233-43, 2006). The normal LDH value range is 105-333 IU/L (international units per liter).

LDH levels can be measured using any suitable test or assay, such as those described by Ferri F F, ed. Ferri's Clinical Advisor 2014. Philadelphia: Pa: Elsevier Mosby; 2014: Section IV-Laboratory tests and interpretation of results. LDH concentration can be measured in various samples obtained from a patient, in particular, serum samples. As used herein, the term “sample” refers to biological material from a subject. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are known in the art and can be readily adapted to obtain a sample that is compatible with the method utilized.

In one embodiment, the treatments described herein result in a normalization of LDH levels. In another embodiment, patients treated according to the disclosed methods experience reductions in LDH levels to near normal levels or to within 10%, or within 20% above what is considered the normal level (e.g., within 105-333 IU/L (international units per liter). In some embodiments, the treatment results in a percentage change in LDH levels relative to baseline. In some embodiments, the treatment results in a percentage change in LDH levels relative to baseline at Week 10 and Week 52.

In another embodiment, the patient's LDH levels are normalized throughout maintenance period of treatment. In another embodiment, the treated patient's LDH levels are normalized at least at least 95% of the time while on the maintenance period of treatment. In another embodiment, the treated patient's LDH levels are normalized at least at least 90%, 85% or 80% of the time while on the maintenance period of treatment. In one embodiment, the patient's LDH levels are 21.5 fold above the upper limit of normal (LDH≥1.5×ULN) prior to initiating treatment. In another embodiment, the treatment results in a normalization of LDH levels by at least day 24 of treatment. In one embodiment, patients treated according to the disclosed methods experience reductions in LDH levels to within normal levels or to within 10%, 20%, 30%, 40% or within 50% below what is considered the upper limit of normal level (e.g., within 105-333 IU/L (international units per liter). In one embodiment, the patient's LDH levels are 21.5 fold above the upper limit of normal (LDH ≥1.5×ULN) prior to initiating treatment. In one embodiment, the treatment results in LDH levels less than 2× upper limit of normal (ULN).

Provided herein are systems, devices, and methods for treating aHUS in a pediatric patient comprising administering to the patient an anti-C5 antibody, or antigen-binding fragment thereof. Symptoms of aHUS, especially in pediatric patients, are described in WO2019236345, including, the clinical trial identified as ALXN1210-aHUS-312 (NCT03131219), pediatric aHUS study), the disclosure(s) in which are incorporated by reference herein. They include, but are not limited to, endothelial damage caused by thrombotic microangiopathy (TMA), including, life-threatening manifestations thereof, e.g., kidney failure and extrarenal tissue damage. See, e.g., Tanaka et al. (Pediatr Nephrol. 2021; 36(4): 889-898), the pertinent disclosure therein is incorporated by reference herein.

### Kits

Also provided herein are kits that include a pharmaceutical composition containing an anti-C5 antibody or antigen-binding fragment thereof, such as ravulizumab, and a pharmaceutically acceptable carrier, in a therapeutically effective amount adapted for use in the preceding methods. The kits can also optionally include instructions, e.g., comprising administration schedules, to allow a practitioner (e.g., a physician, nurse or patient) to administer the composition contained therein to administer the composition to a patient having PNH or aHUS. The kit also can include a syringe.

Kits can optionally include multiple packages of the single-dose pharmaceutical compositions each containing an effective amount of the anti-C5 antibody, or antigen-binding fragment thereof, for a single administration in accordance with the methods provided above. Instruments or devices necessary for administering the pharmaceutical composition(s) also may be included in the kits. A kit may provide one or more pre-filled syringes containing an amount of the anti-C5 antibody or antigen-binding fragment thereof.

In some embodiments, provided herein is a kit comprising an on-body delivery system (OBDS) described herein and instructions for using the OBDS for treatment of PNH or aHUS in a pediatric human patient in accordance with any of the methods described herein. In some embodiments, the OBDS is configured for administration of an effective amount of an anti-C5 antibody, or antigen-binding fragment thereof. In some embodiments, the patient is complement inhibitor treatment-naïve or eculizumab-experienced. In some embodiments, the patient is ravulizumab-experienced.

In some embodiments, provided herein is a kit comprising a needle syringe described herein and instructions for using the needle syringe for treatment of PNH or aHUS in a pediatric human patient in in accordance with any of the methods described herein. In some embodiments, the needle syringe is configured for administration of an effective amount of an anti-C5 antibody, or antigen-binding fragment thereof. In some embodiments, the patient is ≥10 to <20 kg of body weight and is complement inhibitor treatment-naïve, eculizumab-experienced, or ravulizumab-experienced.

In some embodiments, provided herein is a kit for treating PNH or aHUS in a pediatric human patient, wherein the patient is complement inhibitor treatment-naïve or eculizumab-experienced, including: (a) a dose of an anti-C5 antibody, or antigen-binding fragment thereof, and (b) instructions for using the anti-C5 antibody, or antigen-binding fragment thereof, in accordance with any of the methods described herein. In some embodiments of the foregoing aspects, the anti-C5 antibody, or antigen-binding fragment thereof, is provided in a 3 mL vial including Type I borosilicate glass for (a) intravenous administration and (b) subcutaneous administration to a patient weighing ≥10 to <20 kg.

In some embodiments, provided herein is a kit for treating PNH or aHUS in a pediatric human patient, wherein the patient is ravulizumab-experienced, including: (a) a dose of an anti-C5 antibody, or antigen-binding fragment thereof, and (b) instructions for using the anti-C5 antibody, or antigen-binding fragment thereof, in accordance with any of the methods described herein.

In some embodiments, a kit described herein may be used to provide an anti-C5 antibody, or antigen-binding fragment thereof, which is administered subcutaneously once per week at a dose of: (i) 150 mg to a patient weighing ≥10 to <20 kg, (ii) 245 mg to a patient weighing ≥20 to <40 kg, or (iii) 490 mg to a patient weighing ≥40 kg. In some embodiments, the pediatric human patient is less than 21 years of age. In some embodiments, the pediatric human patient is between 2 and 18 years of age. In some embodiments, the anti-C5 antibody, or antigen-binding fragment thereof, is configured for subcutaneous administration to a site selected from the patient's arm, abdomen, and thigh.

Any suitable anti-C5 antibody, or antigen-binding fragment thereof, may be included in a kit as provided herein, including any suitable anti-C5 antibody or antigen-binding fragment thereof disclosed herein. In some embodiments, the anti-C5 antibody, or antigen-binding fragment thereof, comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6. In some embodiments, the anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering. In some embodiments, the anti-C5 antibody, or antigen-binding fragment thereof, comprises a heavy chain variable region comprising the amino acid sequence of SEQ ID NO: 12 and a light chain variable region comprising the amino acid sequence of SEQ ID NO 8. In some embodiments, the heavy chain variable region comprises an N-terminal pyroglutamate residue. In some embodiments, the anti-C5 antibody, or antigen-binding fragment thereof, further comprises a heavy chain constant region comprising the amino acid sequence of SEQ ID NO: 13. In some embodiments, the anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### Example

### Example 1: A Phase 3, Open-Label, Single-Arm, Multicenter Study to Evaluate the Pharmacokinetics, Pharmacodynamics, Activity, and Safety of Ravulizumab Administered Subcutaneously in Pediatric Participants (2 to <18 Years of Age) with Paroxysmal Nocturnal Hemoglobinuria (PNH) or Atypical Hemolytic Uremic Syndrome (aHUS)

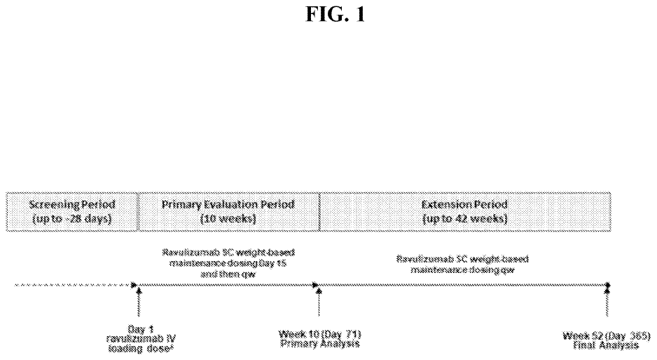

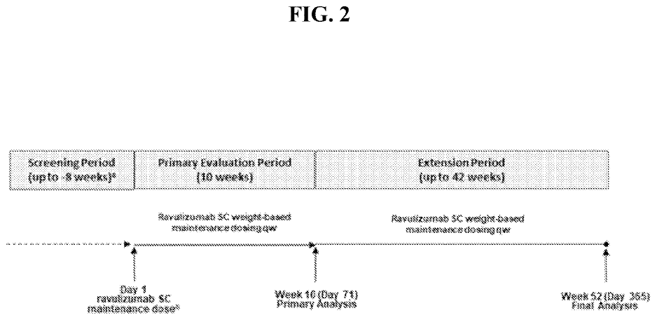

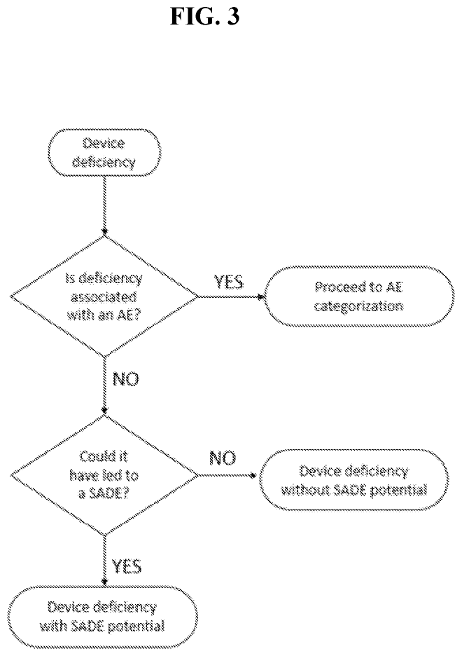

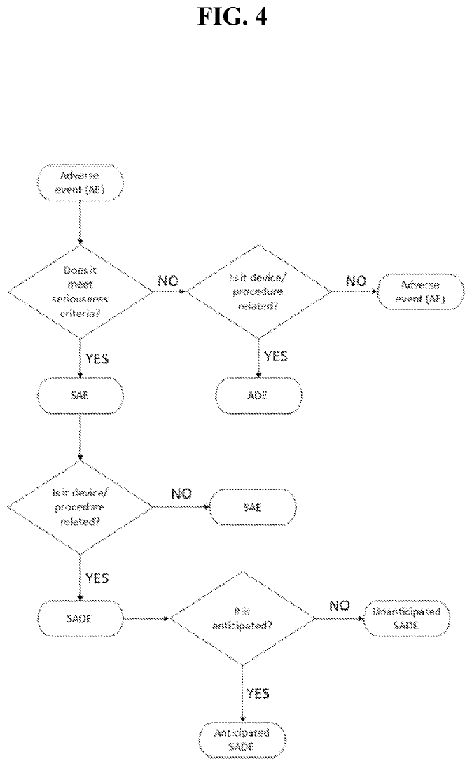

This example describes a Phase 3, open-label, single-arm, multicenter study to evaluate the safety and efficacy of ravulizumab administered subcutaneously in pediatric human participants (2 to <18 years of age) with PNH or aHUS. The study schematic is shown in FIGS. 1 and 2.

### BACKGROUND

PNH is an ultra-rare, progressive, debilitating, and life-threatening disease characterized by complement-mediated intravascular hemolysis, thrombosis, and bone marrow failure. Ravulizumab was approved as an intravenous (IV) treatment for pediatric patients with PNH based on data from controlled clinical studies in adult patients with additional pharmacokinetic (PK), efficacy, and safety data acquired in a dedicated study (ALXN1210-PNH-304) in pediatric patients 9 to <18 years of age. Use in pediatric patients with PNH <9 years of age and body weight <30 kg was based on extrapolation of PK/pharmacodynamic (PD), efficacy, and safety data from aHUS and PNH clinical studies.

aHUS is a rare, progressive, and life-threatening disorder characterized by hemolytic anemia, thrombocytopenia, and acute renal injury, although extra-renal complications occur frequently. The approval for ravulizumab IV for the treatment of pediatric patients with aHUS was based on data from controlled clinical studies in adults with additional PK, efficacy, and safety data acquired in a dedicated study (ALXN1210-aHUS-312) in pediatric patients 10 months to <18 years of age.

Alexion has developed a subcutaneous (SC) formulation of ravulizumab as an alternative to the IV route of administration. The PK, PD, efficacy, and safety of ravulizumab SC was demonstrated in a Phase 3 study (ALXN1210-PNH-303) in adult patients with PNH who received ravulizumab via an on-body delivery system (OBDS) comprised of a prefilled cartridge containing ravulizumab co-packaged with an on-body injector (OBI).

In various embodiments, the formulations or devices of the present disclosure maintain ravulizumab drug concentrations above the PK target (175 μg/mL), resulting in complete terminal complement inhibition (serum free C5<0.5 μg/mL) and control of disease in pediatric patients with PNH or aHUS, regardless of prior treatment with complement inhibitors.

Using the formulations and devices of the disclosure the characteristic PK and PD of ravulizumab SC administered via an OBI (patient ≥20 kg) or via syringe (patient <20 kg) in patients 2 to <18 years of age with either PNH or aHUS during a 10-week Primary Evaluation Period is evaluated for the attainment of the estimands and/or endpoints provided below.

Objectives and Estimands and/or Endpoints:

| Objectives | Estimands and/or Endpoints |
|---|---|
| Primary (Both Cohorts) | |
| To characterize the PK and PD of | Primary PK Estimand: |
| treatment with ravulizumab SC in | Treatment: Ravulizumab SC |
| pediatric participants with PNH or | Population: Pediatric participants with PNH or aHUS who |
| aHUS | are included in the PK Analysis Set |
| | Variables: Ravulizumab concentrations at Day 1 postdose, |
| | Day 15 predose, Day 15 postdose, and Day 71 predose |
| | Intercurrent events (IEs): |
| | IE1: Discontinuation of study intervention prior to the |
| | end of the Primary Evaluation Period (Day 71) |
| | IE2: Initiation of specific disallowed medication or |
| | therapy prior to the end of the Primary Evaluation |
| | Period (Day 71) |
| | Data collected for participants after IE1 or IE2 will not be |
| | used for the primary PK analysis. |
| | Summary measures: Summary statistics of ravulizumab |
| | concentrations at Day 1 postdose, Day 15 predose, Day 15 |
| | postdose, and Day 71 predose |
| | Primary PD Estimand: |
| | Treatment: Ravulizumab SC |
| | Population: Pediatric participants with PNH or aHUS who |
| | are included in the PD Analysis Set |
| | Variables: Free C5 concentrations at Day 1 postdose, Day |
| | 15 predose, Day 15 postdose, and Day 71 predose |
| | Intercurrent events (IEs): |
| | IE1: Discontinuation of study intervention prior to the |
| | end of the Primary Evaluation Period (Day 71) |
| | IE2: Initiation of specific disallowed medication or |
| | therapy prior to the end of the Primary Evaluation |
| | Period (Day 71) |
| | Data collected for participants after IE1 or IE2 will not be |
| | used for the primary PD analysis. |
| | Summary measures: Summary statistics of serum free C5 |
| | concentrations at Day 1 postdose, Day 15 predose, Day 15 |
| | postdose, and Day 71 predose |
| Secondary Endpoints | |
| PK and PD (Both Cohorts) | |
| To characterize the PK and PD of | Serum ravulizumab concentrations over time through Week |
| treatment with ravulizumab SC in | 52 |
| pediatric participants with PNH or | Absolute values, change from baseline, and percentage |
| aHUS | change from baseline for free serum C5 concentrations over |
| | time through Week 52 |
| Efficacy (PNH Cohort Only) | |
| To assess the efficacy of | Percentage change from baseline in LDH to Week 10 and |
| treatment with ravulizumab SC in | Week 52 |
| pediatric participants with PNH | Incidence of BTH through Week 10 and Week 52 |
| | Achievement of transfusion avoidance through Week 10 and |
| | Week 52 |
| | Achievement of stabilized hemoglobin through Week 10 and |
| | Week 52 |
| | Change from baseline in PNH RBC clone size at Week 52 |
| Efficacy (aHUS Cohort Only) | |
| To assess the efficacy of | Dialysis requirement status through Week 10 and Week 52 |
| treatment with ravulizumab SC in | Observed value and change from baseline in eGFR through |
| pediatric participants with aHUS | Week 10 and Week 52 |
| | Observed value and change from baseline in serum creatinine |
| | through Week 10 and Week 52 |
| | Observed value and change from baseline in hematologic |
| | parameters through Week 10 and Week 52: |
| | Platelets |
| | LDH |
| | Hemoglobin |
| Health-related QoL (Both Cohorts) | |
| To assess the effect of treatment | Change from baseline in patient-reported fatigue, as |
| with ravulizumab SC on fatigue | measured by Pediatric FACIT-Fatigue (participants ≥8 years |
| and QoL based on patient- | of age) to Week 10 and Week 52 |
| reported and caregiver-reported | Change from baseline in PedsQL 4.0 Generic Core Scale to |
| outcomes in pediatric participants | Week 10 and Week 52 |
| with PNH or aHUS | |
| Safety (Both Cohorts) | |
| To evaluate safety of | Incidence of AEs and SAEs |
| ravulizumab SC in pediatric | Incidence of ADEs and serious ADEs in participants treated |
| participants with PNH or aHUS | with ravulizumab SC via OBI |
| Device Performance (Both Cohorts) | |
| To assess performance of | Reported outcome of attempted full-dose administration via |
| ravulizumab OBDS in pediatric | OBI |
| participants with PNH or aHUS | Reported device deficiencies/complaints and associated |
| | device investigations |
| Immunogenicity (Both Cohorts) | |
| To assess immunogenicity of | ADA incidence, response categories and titer in study |
| ravulizumab SC in pediatric | participants treated with ravulizumab SC for the duration of |
| participants with PNH or aHUS | the study |

Abbreviations: ADA = antidrug antibody;
ADE = adverse device effect;
AE = adverse event;
aHUS = atypical hemolytic uremic syndrome;
BTH = breakthrough hemolysis;
C5 = complement component 5;
eGFR = estimated glomerular filtration rate;
FACIT-Fatigue = Functional Assessment of Chronic Illness Therapy-Fatigue;
IE = intercurrent event;
LDH = lactate dehydrogenase;
OBDS = on-body delivery system;
OBI = on-body injector;
PD = pharmacodynamic;
PedsQL = Pediatric Quality of Life Inventory;
PK = pharmacokinetic;
PNH = paroxysmal nocturnal hemoglobinuria;
QoL = quality of life;
SC = subcutaneous;
SAE = serious adverse event

### Overall Design

This is a Phase 3, open-label, multicenter study to evaluate the PK and PD of ravulizumab SC in pediatric participants (2 to <18 years of age) with documented PNH or aHUS who are either complement inhibitor treatment-naïve or have previously received complement inhibitor treatment. Participants who have previously received complement inhibitor treatment must be clinically stable and have been previously treated with eculizumab or ravulizumab for at least 90 days prior to Screening.

The study consists of a Screening Period (up to 4 weeks for complement inhibitor treatment-naïve or eculizumab-experienced participants, up to 8 weeks [4 weeks for participants <20 kg] for ravulizumab-experienced participants), a 10-week Primary Evaluation Period, and an Extension Period (up to 42 weeks). All participants will be followed for safety for 8 weeks after the last dose of study intervention. Thus, the total treatment duration is up to 1 year (52 weeks) and the total study duration is up to 59 weeks (note: last dose of study intervention is on Week 51).

Approximately 22 participants will be enrolled in order to obtain at least 20 pediatric participants (2 to <18 years of age) who are evaluable for the PK/PD primary analyses will be enrolled and treated. The minimum number of participants for each age category is as follows: at least 3 participants in the 2 to <6 years age group; at least 7 participants in the 6 to <12 years age group; and at least 10 participants in the 12 to <18 years age group.

All participants will be screened for eligibility during the Screening Period. Complement inhibitor treatment-naïve or eculizumab-experienced participants will be screened for eligibility during the Screening Period for up to 4 weeks (28 days). Participants who are ravulizumab-experienced will be screened for eligibility during the Screening Period for up to 8 weeks (56 days) for participants weighing ≥20 kg and up to 4 weeks (28 days) for participants weighing <20 kg.

Eligible complement inhibitor treatment-naïve or eculizumab-experienced participants will receive a weight-based ravulizumab IV loading dose on Day 1. For eculizumab-experienced participants, Day 1 of study intervention will occur 2 weeks (14±2 days) from the participant's last eculizumab infusion. During the Primary Evaluation Period, participants will receive weight-based ravulizumab SC maintenance dosing on Day 15 and every week (qw) thereafter.

For ravulizumab-experienced participants, Day 1 of study intervention will occur 8 weeks (56±5 days) from the participant's last ravulizumab infusion for participants weighing ≥20 kg or 4 weeks (28±2 days) from the participant's last ravulizumab infusion for participants weighing <20 kg. During the Primary Evaluation Period, ravulizumab-experienced participants will receive a weight-based ravulizumab SC maintenance dose on Day 1, followed by weight-based maintenance doses of ravulizumab SC qw thereafter.

During the Extension Period, all participants will continue to receive weight-based maintenance doses of ravulizumab SC qw through the end of the Extension Period.

If a participant discontinues study intervention, the participant will be withdrawn from the study and encouraged to complete the Early Discontinuation (ED) Visit as soon as possible.

A Safety Follow-up Visit will be conducted remotely 8 weeks (56±7 days) after the last dose of ravulizumab (SC or IV) to collect information on concomitant medications, nonpharmacologic therapies and procedures, adverse events (AEs), and adverse device effects (ADEs).

The end of the study is defined as the date the last participant completes the last visit (including follow-up) as shown in the Schedule of Activities.

This is a single group treatment study with no masking.

Number of Participants: Approximately 22 participants will be enrolled for the PK/PD primary analyses.

### Intervention Groups and Duration:

Eligible participants who are complement inhibitor treatment-naïve or eculizumab-experienced will receive a weight-based ravulizumab IV loading dose on Day 1 and weight-based ravulizumab SC maintenance dosing on Day 15 and qw thereafter for a total of 52 weeks of study treatment. Ravulizumab-experienced participants will receive a weight-based ravulizumab SC maintenance dose on Day 1, followed by weight-based maintenance doses of ravulizumab SC qw. Dosages will be based on the participant's body weight, as shown in the tables below:

Weight-based Ravulizumab Dosing Regimen for Complement
Inhibitor Treatment-Naïve Participants
or Eculizumab-Experienced Participants

| | | | Ravulizumab SC | |
|---|---|---|---|---|
| | Ravulizumab | | Maintenance | |
| | IV | | Dosing Regimen | |
| Body Weight | Loading Dose | | (Day 15 and | |
| Range (kg) | (Day 1) | | thereafter) | Delivery Method |
| ≥10 to <20 | 600 | mg | 150 mg qw | 1.5 mL via syringe |
| ≥20 to <30 | 900 | mg | 245 mg qw | 1 ravulizumab OBDS |
| ≥30 to <40 | 1200 | mg | | kit |
| ≥40 to <60 | 2400 | mg | 490 mg qw | 2 ravulizumab OBDS |
| ≥60 | 2700 | mg | | kits |

Abbreviations:
IV = intravenous;
OBDS = on-body delivery system;
qw = every week;
SC = subcutaneous

Weight-based Ravulizumab Dosing Regimen
for Ravulizumab-Experienced Participants

| | Ravulizumab SC | |
|---|---|---|
| Body Weight | Maintenance Dosing Regimen | |
| Range (kg) | (Day 1 and thereafter) | Delivery Method |
| ≥10 to <20 | 150 mg qw | 1.5 mL via syringe |
| ≥20 to <30 | 245 mg qw | 1 ravulizumab OBDS kit |
| ≥30 to <40 | | |
| ≥40 to <60 | 490 mg qw | 2 ravulizumab OBDS kits |
| ≥60 | | |

Abbreviations:
OBDS = on-body delivery system;
qw = every week;
SC = subcutaneous

### Ethical Considerations and Benefit-Risk Assessment

This study will be conducted as specified in this protocol and in accordance with the established ethical guidelines.

### Schema: See FIGS. 1 and 2.

Note: All participants will be followed for safety for 8 weeks after the last dose of study intervention.

Weight-based dosing regimen as follows:

| Ravulizumab IV Loading Dose | Ravulizumab SC Maintenance Dosing Regimen |
|---|---|
| ≥10 to <20 kg = 600 mg | ≥10 to <20 kg = 150 mg qw (via SC injection[s]) |
| ≥20 to <30 kg = 900 mg | ≥20 to <30 kg = 245 mg qw (via ravulizumab OBDS) |
| ≥30 to <40 kg = 1200 mg | ≥30 to <40 kg = 245 mg qw (via ravulizumab OBDS) |
| ≥40 to <60 kg = 2400 mg | ≥40 to <60 kg = 490 mg qw (via ravulizumab OBDS) |
| ≥60 kg = 2700 mg | ≥60 kg = 490 mg qw (via ravulizumab OBDS) |

^{a} For eculizumab-experienced participants, the ravulizumab IV loading dose will be administered 2 weeks (14 ± 2 days) from the participant's last eculizumab infusion
Abbreviations:
IV = intravenous;
OBDS = on-body delivery system;
qw = every week;
SC = subcutaneous

Note: All participants will be followed for safety for 8 weeks after the last dose of study intervention.

Weight-based dosing regimen as follows:

Ravulizumab SC Maintenance Dosing Regimen

≥10 to <20 kg = 150 mg qw (via SC injection)
≥20 to <40 kg = 245 mg qw (via ravulizumab OBDS)
≥40 kg = 490 mg qw (via ravulizumab OBDS)
^{a} For participants weighing <20 kg, the Screening Period will be up to 4 weeks prior to Day 1.
^{b} For ravulizumab-experienced participants, the Day 1 ravulizumab SC maintenance dose will be administered approximately 8 weeks (56 ± 5 days) from the participant's last ravulizumab IV dose for participants weighing ≥20 kg or 4 weeks (28 ± 2 days) from the participant's last ravulizumab infusion for participants weighing <20 kg.
Abbreviations:
IV = intravenous;
OBDS = on-body delivery system;
qw = every week;
SC = subcutaneous

### 1.1. Schedule of Activities

### Schedules of Activities are Provided as Follows:

Screening Through End of the Primary Evaluation Period (Week 10 Visit) is provided in Table 1.
Extension Period: Week 14 Visit Through Week 52 Visit is provided in Table 2.

TABLE 1
Schedule of Activities: Screening Through the End of the Primary Evaluation Period (Week 10 Visit)

| Period | | | | | | | | | | | | | |
|---|---|---|---|---|---|---|---|---|---|---|---|---|---|
| Screening | Primary Evaluation Period | | | | | | | | | | | | |
| Week | | | | | | | | | | | | | Safety |
| Up to 4 W or 8 W^{a} | | W 1 | W 2 | W 3 | W 4 | W 5 | W 6 | W 7 | W 8 | W 9 | W 10 | | Follow- |
| −56 to −1 or −28 to −1^{a} | 1^{b} | 8 ± 1 | 15 ± 1 | 22 ± 1 | 29 ± 1 | 36 ± 1 | 43 ± 1 | 50 ± 1 | 57 ± 1 | 64 ± 1 | 71^{c} ± 1 | ED^{d} | up^{e} |

Visit may be done via phone/video call

| | | | | | | |
|---|---|---|---|---|---|---|
| X^{f} | X^{f} | X^{f} | X^{f} | X^{f} | X | Notes |

General Assessments/Procedures

| Informed consent | X | | | | | | | | |
|---|---|---|---|---|---|---|---|---|---|
| Confirm inclusion/exclusion | X | X | | | | | | | Confirm eligibility prior |
| | | | | | | | | | to first dose of study |
| | | | | | | | | | intervention; |
| | | | | | | | | | participants may be |
| | | | | | | | | | rescreened once |
| Demographics and medical | X | | | | | | | | |
| history | | | | | | | | | |
| Prior medications and | X | | | | | | | | |
| procedures | | | | | | | | | |
| Meningococcal vaccination | X | Completion of vaccination series and re-vaccination | | | | | | | See Section 4.18.5 for |
| | | according to national and local guidelines | | | | | | | vaccination |
| | | | | | | | | | requirements |
| Hib and S pneumoniae | X | Completion of vaccination series and re-vaccination | | | | | | | |
| vaccination | | according to national and local guidelines | | | | | | | |
| Specialty screening labs^{g} | X | | | | | | | | ADAMTS13, Coombs |
| (aHUS cohort only) | | | | | | | | | test, and ST-HUS |
| Pregnancy test (WOCBP | X | X | X | | | | X | X | Serum test required at |
| only) | | | | | | | | | Screening, Day 71, |
| | | | | | | | | | and ED; urine test all |
| | | | | | | | | | other visits |
| Weight^{h} | X | X | X | X | X | X | X | X | |
| Height | X | X | | | | | X | X | |
| Head circumference (only | X | X | X | X | X | X | X | X | |
| required for participants ≤3 | | | | | | | | | |
| years of age at time of | | | | | | | | | |
| assessment) | | | | | | | | | |

General Assessments/Procedures (continued)

| Vital signsⁱ | X | X | | X | | X | | X | | X | | X | X | | |
|---|---|---|---|---|---|---|---|---|---|---|---|---|---|---|---|
| Physical examination | X | | | | | | | | | | | | | | |
| Abbreviated physical | | X | | X | | X | | X | | X | | X | X | | |
| examination^{j} | | | | | | | | | | | | | | | |
| PNH clone size^{k} (PNH cohort only) | X | X | | | | | | | | | | | X | | |
| Safety 12-lead ECG^{l} | X | | | | | | | | | | | X | X | | |
| Concomitant medications, | | Continual monitoring | | | | | | | | | | | | X | |
| nonpharmacologic therapies, | | | | | | | | | | | | | | | |
| and procedures | | | | | | | | | | | | | | | |
| AEs/ADEs | | Continual monitoring | | | | | | | | | | | | X | |
| Infusion/injection site | | X | | X | | X | | X | | X | | X | | | |
| evaluation^{m} | | | | | | | | | | | | | | | |
| Dispense Participant Safety | X | | | | | | | | | | | | | | Instruct participants to |
| Card | | | | | | | | | | | | | | | carry safety card at all |
| | | | | | | | | | | | | | | | times |
| Review Participant Safety | | X | X | X | X | X | X | X | X | X | X | X | X | X | Participants must |
| Card | | | | | | | | | | | | | | | carry the card at all |
| | | | | | | | | | | | | | | | times until 8 months |
| | | | | | | | | | | | | | | | after the last dose of |
| | | | | | | | | | | | | | | | study intervention (see |
| | | | | | | | | | | | | | | | Section 4.21.1) |
| Clinical laboratory tests | X | Xⁿ | | X | | X | | X | | X | | X | X | | See Section 5.8 |

General Assessments/Procedures (continued)

| Breakthrough hemolysis^{o} | | Continual monitoring | | | | |
|---|---|---|---|---|---|---|
| (PNH cohort only) | | | | | | |
| Record transfusions and | X | Continual monitoring | | | | |
| transfusion parameters | | | | | | |
| (PNH cohort only) | | | | | | |
| Dialysis requirement status | X | Continual monitoring | | | | |
| (aHUS cohort only) | | | | | | |
| Pediatric FACIT-Fatigue | X | X | X | X | X | Administer QoL |
| questionnaire (participants ≥8 | | | | | | assessments prior to |
| years of age at time of | | | | | | performing other study |
| consent) | | | | | | |
| PedsQL 4.0 Generic Core | | X | X | X | X | |
| Scale - Child Self-report and | | | | | | |
| Parent Proxy report | | | | | | |

PK/PD/Immunogenicity Assessments

| | | | | | | | |
|---|---|---|---|---|---|---|---|
| Predose blood samples for | X | X | X | X | X | X | Collect anytime at ED |
| PK/PD^{p} | | | | | | | Visit |
| Postdose blood samples for | X | X | | | X | | |
| PK/PD^{q} | | | | | | | |
| Predose blood sample for | X | | | X | X | X | ADA samples should |
| ADA^{p} | | | | | | | always be collected |
| | | | | | | | prior to study |
| | | | | | | | intervention |
| | | | | | | | administration. Collect |
| | | | | | | | anytime at ED Visit |

Study Intervention for Complement Inhibitor Treatment-
naïve Participants or Eculizumab-experienced Participants

| Ravulizumab IV loading | X | | | | | | | | | | | Administer after all |
|---|---|---|---|---|---|---|---|---|---|---|---|---|
| dose administration | | | | | | | | | | | | other required |
| Ravulizumab SC | | | X | X | X | X | X | X | X | X | X^{c} | tests/procedures |
| maintenance dose | | | | | | | | | | | | |
| administration^{r} | | | | | | | | | | | | |
| Caregiver/participant training | | | X | Performed as needed^{s} | | | | | | | | |
| on dose administration^{s} | | | | | | | | | | | | |
| Device performance and | | Continual monitoring | | | | | | | | | | |
| product_complaints | | | | | | | | | | | | |
| Treatment compliance | | Record at each home dosing time point | | | | | | | | | | |
| monitoring via e-diary | | | | | | | | | | | | |

Study Intervention for Ravulizumab-experienced Participants

| Ravulizumab SC | X | X | X | X | X | X | X | X | X | X | X^{c} | Administer after all |
|---|---|---|---|---|---|---|---|---|---|---|---|---|
| maintenance dose | | | | | | | | | | | | other required |
| administration^{r} | | | | | | | | | | | | tests/procedures |
| Caregiver/participant training | X | Performed as needed^{s} | | | | | | | | | | |
| on dose administration^{s} | | | | | | | | | | | | |
| Device performance and | Continual monitoring | | | | | | | | | | | |
| product_complaint^{s} | | | | | | | | | | | | |
| Treatment compliance | Record at each home dosing time point | | | | | | | | | | | |
| monitoring via e-diary | | | | | | | | | | | | |

Note:
All assessments must be performed prior to administration of study intervention on dosing days, unless otherwise specified.
^{a}Complement inhibitor treatment-naïve or eculizumab-experienced participants will be screened for eligibility during the Screening Period for up to 4 weeks (28 days). Participants who are ravulizumab-experienced will be screened for eligibility during the Screening Period for up to 8 weeks (56 days) for participants weighing ≥20 kg and up to 4 weeks (28 days) for participants weighing <20 kg.
^{b}For eculizumab-experienced participants, Day 1 will occur 2 weeks (14 ± 2 days) from the participant's last infusion of eculizumab. For ravulizumab-experienced participants, Day 1 will occur 8 weeks (56 ± 5 days) from the participant's last infusion of ravulizumab (or 4 weeks [28 ± 2 days] for participants weighing <20 kg).
^{c}Day 71 assessments for the Primary Evaluation Period must be performed predose on Day 71. Dosing on Day 71 is the start of the Extension Period; participants will continue to receive maintenance doses of ravulizumab SC thereafter during the Extension Period (Table 2).
^{d}For participants who discontinue study intervention, an ED visit will be completed as soon as possible.
^{e}A Safety Follow-up Visit will be performed 8 weeks (56 ± 7 days) following the participant's last dose of study intervention (ravulizumab IV or SC) to collect information on concomitant medications, nonpharmacologic therapies and procedures, AEs, and ADEs.
^{f}Site staff will contact participant/caregiver via phone/video call at scheduled dosing times to ensure participant/caregiver is queried about study intervention dose administration and device condition.
^{g}For eculizumab-experienced participants with aHUS or ravulizumab-experienced participants with aHUS, historical test results via chart review may be utilized.
^{h}Weight will be measured predose. Dosages will be based on the participant's weight collected at the protocol-required body weight measurement. If the protocol-required body weight measurement is not available, the weight recorded at the previous protocol-required body weight measurement will be used.
ⁱVital sign measurements include systolic and diastolic BP, heart rate, respiratory rate, and body temperature. On dosing days, vital signs will be taken predose and after the participant has been resting for at least 5 minutes.
^{j}Abbreviated physical examination consists of a body system relevant examination based upon the Investigator's (or qualified designee) judgment and participant symptoms. At least 1 body system must be checked for an abbreviated examination.
^{k}White blood cell (granulocyte and monocyte) and RBC clone size measured by high-sensitivity flow cytometry.
^{l}Participants must be supine for approximately 5 to 10 minutes before ECG collection and remain supine but awake during ECG collection.
^{m}New or worsening abnormalities will be reported as AEs.
ⁿFor participants entering the study on eculizumab or ravulizumab therapy, screening LDH will be obtained within 24 hours prior to a scheduled eculizumab/ravulizumab dose.
^{o}If a suspected event of breakthrough hemolysis occurs, blood samples for LDH, PK, PD, and ADA parameters will be collected and sent to the central laboratory for analysis. If the suspected event of breakthrough hemolysis does not occur at a scheduled visit, an unscheduled visit will occur for evaluation of the participant and collection of the required blood samples.
^{p}All predose samples will be collected within 30 minutes prior to the dose of study intervention.
^{q}Postdose samples will be collected within 60 minutes after completion of the dose of study intervention.
^{r}For doses that coincide with study visit days, ravulizumab SC may be self-administered (participants ≥12 years of age) or administered by a caregiver (participants 2 to <18 years of age) or by trained study site personnel. All other doses may be self-administered at home.
^{s}A qualified member of the site study team will provide initial (and ongoing as appropriate) training on how to properly self-administer ravulizumab SC to caregivers and participants ≥12 years of age. If a participant transitions from SC administration via syringe to SC administration via the ravulizumab OBDS during the study, the site study team will provide training for ravulizumab OBDS administration.
Abbreviations:
ADA = antidrug antibody;
ADAMTS13 = a disintegrin and metalloproteinase with a thrombospondin type 1 motif, member 13;
ADE = adverse device effect;
AE = adverse event;
aHUS = atypical hemolytic uremic syndrome;
BP = blood pressure;
ECG = electrocardiogram;
ED = early discontinuation;
e-diary = electronic diary;
eGFR = estimated glomerular filtration rate;
FACIT-Fatigue = Functional Assessment of Chronic Illness Therapy-Fatigue;
Hib = Haemophilus influenzae type b;
IV = intravenous;
LDH = lactate dehydrogenase;
PD = pharmacodynamic;
PedsQL = Pediatric Quality of Life Inventory;
PK = pharmacokinetic;
PNH = paroxysmal nocturnal hemoglobinuria;
qw = every week;
RBC = red blood cell;
SC = subcutaneous;
ST-HUS = Shiga toxin-related hemolytic uremic syndrome;
WOCBP = women of childbearing potential

TABLE 2
Schedule of Activities - Extension Period Week 14 Visit Through Week 52 Visit

| Period | | | | | | | |
|---|---|---|---|---|---|---|---|
| Extension Period | | | | | | | |
| Week | | | | | | | |
| W 14 | W 18 | W 26 | W 34 | W 42 | W 52 | | |
| Days | | | | | | | |
| 99 ± | 127 ± | 183 ± | 239 ± | 295 ± | 365 ± | | Safety |
| 1 | 1 | 1 | 1 | 1 | 1 | ED^{a} | Follow-up^{b} |

Visit may be done via phone/video call

| | |
|---|---|
| X | Notes |

General Assessments/Procedures

| Weight and height^{c} | X | X | X | X | X | X | X | | |
|---|---|---|---|---|---|---|---|---|---|
| Head circumference (only required for | X | X | X | X | X | X | X | | |
| participants ≤3 years of age at time of | | | | | | | | | |
| assessment) | | | | | | | | | |
| Vital signs^{d} | X | X | X | X | X | X | X | | |
| Abbreviated physical examination^{e} | X | X | X | X | X | X | X | | |
| PNH clone size^{(PNH cohort only)} | | | | | | X | X | | |
| Pregnancy test (WOCBP only) | X | X | X | X | X | X | X | | Serum test required at Week 52 and |
| | | | | | | | | | ED Visit (if applicable); local urine test |
| | | | | | | | | | all other visits |
| Safety 12-lead ECG^{f} | | | | | | X | X | | |
| Concomitant medications, nonpharmacologic | Continual monitoring | | | | | | | X | |
| therapies, and procedures | | | | | | | | | |
| AEs/ADEs | Continual monitoring | | | | | | | X | |
| Infusion/injection site evaluation^{g} | X | X | X | X | X | | | | |
| Review Participant Safety Card | X | X | X | X | X | X | X | X | Participants will be instructed to carry |
| | | | | | | | | | the card with them at all times until |
| | | | | | | | | | 8 months after the last dose of study |
| | | | | | | | | | intervention |
| Clinical laboratory tests | X | X | X | X | X | X | X | | Section 5.8 |
| Breakthrough hemolysis^{h} (PNH cohort only) | Continual monitoring | | | | | | | | |
| Record transfusions and transfusion | Continual monitoring | | | | | | | | |
| parameters | | | | | | | | | |
| (PNH cohort only) | | | | | | | | | |
| Dialysis requirement status (aHUS cohort | Continual monitoring | | | | | | | | |
| only) | | | | | | | | | |

General Assessments/Procedures (Continued)

| | | | | | | | |
|---|---|---|---|---|---|---|---|
| Pediatric FACIT-Fatigue questionnaire | X | X | X | X | X | X | Administer QoL assessments prior to |
| (participants ≥8 years of age) | | | | | | | performing other study procedures at |
| PedsQL 4.0 Generic Core Scale - Child Self | X | X | X | X | X | X | each visit |
| report and Parent Proxy report | | | | | | | |

PK/PD/Immunogenicity Assessments

| | | | | | |
|---|---|---|---|---|---|
| Predose blood samples for PK/PDⁱ | X | X | X | X | Collect anytime at Week 52 and ED |
| | | | | | Visit |
| Postdose blood samples for PK/PD^{j} | X | X | | | |
| Predose blood sample for ADAⁱ | X | X | X | X | ADA samples should always be |
| | | | | | collected prior to study intervention |
| | | | | | administration. Collect anytime at |
| | | | | | Week 52 and ED Visit |

Study Intervention (All Participants)

| Ravulizumab SC qw maintenance dose | Weekly dosing | Administer after all other required |
|---|---|---|
| administration^{k} | | tests/procedures at clinic visit. Last |
| | | dose of study intervention |
| | | administration occurs at Week 51. |
| Caregiver/participant training on dose | Performed as needed^{l} | |
| administration^{l} | | |

| | | | |
|---|---|---|---|
| Device performance and product complaints | Continual monitoring | X | X |
| Treatment compliance monitoring via e-dairy | Record at each home dosing time point | | |

Note:
All assessments must be performed prior to administration of study intervention on dosing days, unless otherwise specified.
^{a}For participants who discontinue study intervention, an ED visit will be completed as soon as possible.
^{b}A Safety Follow-up Visit will be performed 8 weeks (±7 days) following the participant's last dose of study intervention (ravulizumab IV or SC) to collect information on concomitant medications, nonpharmacologic therapies and procedures, AEs, and ADEs.
^{c}Weight will be measured predose. Dosages will be based on the participant's weight collected at the protocol-required body weight measurement. If the protocol-required body weight measurement is not available, the weight recorded at the previous protocol-required body weight measurement will be used.
^{d}Vital sign measurements include systolic and diastolic BP, heart rate, respiratory rate, and temperature. On dosing days, vital signs will be taken predose and after the participant has been resting for at least 5 minutes.
^{e}Abbreviated physical examination consists of a body system relevant examination based upon Investigator (or qualified designee) judgment and participant symptoms. At least 1 body system must be checked for an abbreviated examination.
^{f}Participants must be supine for approximately 5 to 10 minutes before ECG collection and remain supine but awake during ECG collection.
^{g}New or worsening abnormalities will be reported as AEs.
^{h}If a suspected event of breakthrough hemolysis occurs, blood samples for LDH, PK, PD, and ADA parameters will be collected and sent to the central laboratory for analysis. If the suspected event of breakthrough hemolysis does not occur at a scheduled visit, an unscheduled visit will occur for evaluation of the participant and collection of the required blood samples.
ⁱPredose samples will be collected within 30 minutes prior to the dose of study intervention.
^{j}Postdose samples will be collected within 60 minutes after completion of the dose of study intervention.
^{k}For doses that coincide with study visit days, ravulizumab SC may be self-administered (participants ≥12 years of age) or administered by a caregiver (participants 2 to <18 years of age) or by trained study site personnel. All other doses may be self-administered at home.
^{l}A qualified member of the site study team will provide initial (and ongoing as appropriate) training on how to properly self-administer ravulizumab SC to caregivers and participants ≥12 years of age. If a participant transitions from SC administration via syringe to SC administration via the ravulizumab OBDS during the study, the site study team will provide training for ravulizumab OBDS administration.
Abbreviations:
ADA = antidrug antibody
ADE = adverse device effect;
AE = adverse event;
aHUS = atypical hemolytic uremic syndrome;
BP = blood pressure;
ED = early discontinuation;
ECG = electrocardiogram;
eGFR = estimated glomerular filtration rate;
FACIT-Fatigue = Functional Assessment of Chronic Illness Therapy-Fatigue;
IV = intravenous;
LDH = lactate dehydrogenase;
PD = pharmacodynamic(s);
PedsQL = Pediatric Quality of Life Inventory;
PK = pharmacokinetic(s);
PNH = paroxysmal nocturnal hemoglobinuria;
qw = every week;
SC = subcutaneous;
WOCBP = women of childbearing potential

### 2. Introduction

### 2.1. Study Overview

A SC formulation of ravulizumab, developed as an alternative to the IV route of administration, was demonstrated to be efficacious and safe in a Phase 3 study in adult patients with PNH (Study ALXN1210-PNH-303). The ravulizumab OBDS, a drug-device combination product comprised of a prefilled cartridge containing ravulizumab co-packaged with an on-body injector (OBI), was used for the SC delivery of ravulizumab in the adult patients.

In this study, pediatric patients will receive ravulizumab SC via the ravulizumab OBDS or via a needle syringe, depending on body weight. The primary objective of this study is to characterize ravulizumab PK and PD following ravulizumab SC dosing in pediatric patients (2 to <18 years of age) with PNH or aHUS during a 10-week Primary Evaluation Period. Both complement-inhibitor naïve and complement inhibitor experiences patients are eligible to participate Patients may have received anti-C5 antibody therapy (eculizumab or ravulizumab) and are enrollable so long as they are clinically stable at the time of Screening. For the purposes of this study, “self-administration” refers to administration by caregivers (for participants 2 to <18 years of age) or by participants (only for participants ≥12 years of age) for patients with PNH or aHUS.

The study is also intended to demonstrate the efficacy, safety, and immunogenicity of ravulizumab SC in pediatric participants with PNH and aHUS.

### 2.2. Background

Paroxysmal nocturnal hemoglobinuria is an ultra-rare, progressive, debilitating, and life-threatening disease characterized by complement-mediated intravascular hemolysis, thrombosis, and bone marrow failure. The approval for ravulizumab IV for the treatment of pediatric patients with PNH was based on data from controlled clinical studies in adult patients with additional PK, efficacy, and safety data acquired in a dedicated study in pediatric patients 9 to <18 years of age (Study ALXN1210-PNH-304). Use in pediatric patients with PNH <9 years of age and body weight <30 kg was based on extrapolation of PK/PD, efficacy, and safety data from aHUS and PNH clinical studies. Atypical hemolytic uremic syndrome is a rare, progressive, and life-threatening disorder characterized by hemolytic anaemia, thrombocytopenia, and acute renal injury, although extra-renal complications occur frequently. The approval for ravulizumab IV for the treatment of pediatric patients with aHUS was based on data from controlled clinical studies in adults with additional PK, efficacy, and safety data acquired in a dedicated study in pediatric patients 10 months to <18 years of age (Study ALXN1210-aHUS-312).

A review of literature on the current state of the art of PNH and aHUS, their epidemiology and management, and the available treatment options was performed to provide the scientific background for this clinical investigation. An unmet medical need exists for drug delivery systems that improve patient treatment experience by offering choice and flexibility, thereby lowering the impact of treatment administration on patient autonomy and quality of life. In addition, the coronavirus disease 2019 (COVID-19) pandemic highlighted the need for continuous patient care for chronic diseases outside of the hospital and clinic settings, especially for highly susceptible and immunocompromised patient populations. To minimize the risk of therapy interruptions, treatment guidelines for certain chronic diseases have been updated during the pandemic, with emphasis on switching to self-administration and SC therapies.

PNH and aHUS are rare diseases that require long-term therapy. The majority of biologics approved for the treatment of these conditions are administered via IV infusion. The SC administration route is shown to be within the state-of-the-art landscape of parenteral delivery of biologics for chronic conditions that require life-long treatments.

### 2.3. Ravulizumab

Ravulizumab (trade name ULTOMIRIS®) is a recombinant, humanized monoclonal antibody with high specificity for human complement component 5 (C5). Ravulizumab administered intravenously (IV) has been shown to achieve immediate, complete, and sustained inhibition of terminal complement and is approved for the treatment of adult patients with AChR-Ab (+) gMG as well as adult and pediatric patients with adult and pediatric patients with PNH and aHUS.

### 2.4. Ravulizumab On-Body Delivery System

The ravulizumab OBDS is a drug-device combination product comprised of a prefilled cartridge containing ravulizumab SC co-packaged with an OBI. The OBI is a compact, sterile, single-use, disposable, electro-mechanical (battery powered, microprocessor controlled), investigational medical device with a 29-gauge integrated needle (contract-manufactured by West Pharmaceuticals, Inc.) designed to be used together with a prefilled stoppered cartridge with a piston and telescopic screw assembly (TSA).

The OBI used in ravulizumab OBDS is based on the West SMARTDOSE® platform, the same base technology utilized with evolocumab (Repatha®) and approved as a combination product in the United States and CE marked in the European Union as a class IIA Medical Device.

The ravulizumab OBDS has been used in the Phase 3 Study ALXN1210-PNH-303 to treat adult participants with PNH.

### 2.5. Benefit/Risk Assessment

### 2.5.1. Risk Assessment

The safety profile for ravulizumab is well established, including in pediatric patients who have been treated in clinical studies for PNH and aHUS. Ravulizumab has been well tolerated and no unexpected safety concerns have been identified based on all currently available data from clinical development programs as well as postmarketing experience. Ravulizumab functions by blocking terminal complement; therefore, participants have increased susceptibility to serious infections, in particular, Neisseria meningitidis. Specific risk mitigation measures available to support the safe use of ravulizumab in participants in this study are described in Table 3.

As with any therapeutic protein, administration of ravulizumab may lead to the development of antidrug antibodies (ADAs). Monitoring of immunogenicity is planned during this study, as described in Section 4.30. Intravenous administration of any investigational product may result in infusion reactions. Management of potential infusion reactions is described in Section 5.9.

TABLE 3
Ravulizumab Important Identified and Potential Risks

| | | |
|---|---|---|
| Potential Risks of | | |
| Clinical | Summary of Data/Rationale for | |
| Significance | Risk | Mitigation Strategy |

Important identified risk

| | | |
|---|---|---|
| Meningococcal | C5 inhibition is known to increase | Participants must be vaccinated against all |
| infection | the susceptibility to infections | available serotypes of N meningitidis (A, C, |
| | caused by N meningitidis. | Y, W135, and B) per the inclusion criteria |
| | | (see also Section 4.18.5). Participants must |
| | | receive the vaccination at least 2 weeks |
| | | before the first dose of study intervention or |
| | | be treated with appropriate antibiotics until |
| | | 2 weeks have passed since their |
| | | vaccination. Each participant will be |
| | | provided with a Participant Safety Card |
| | | with signs and symptoms of meningococcal |
| | | infection, instructions on when to contact a |
| | | healthcare provider, and relevant contact |
| | | information. The Participant Safety Card |
| | | will be reviewed at each visit (Schedule of |
| | | Activities, Section 1.1). |

Important potential risk

| | | |
|---|---|---|
| Serious infection | Apart from the predictable risk of | Increased awareness of healthcare |
| | infection with Neisseria species, | professionals and participants about the |
| | which is well-known and directly | potential risk of serious infection. |
| | related to the mechanism of action | Monitoring for signs and symptoms of |
| | of ravulizumab, the mechanism | serious infections will be conducted as part |
| | that may lead to other serious | of the safety assessments for this study. |
| | infections in patients treated with | |
| | ravulizumab remains unclear. | |
| Immunogenicity | Treatment with any therapeutic | At participant level: Study excludes |
| | protein has the potential to induce | participants with known exposure to other |
| | an immune response. Potential | investigational or marketed biologics, such |
| | clinical consequences may | as mAbs, fusion proteins, bispecific |
| | include hypersensitivity, | molecules, or antibody drug conjugates, |
| | anaphylaxis, or related type of | within 3 months or 5 half-lives (whichever is |
| | reactions or loss of efficacy. | longer) prior to screening. |
| | Fewer than 1% of participants in | Safety monitoring in the study: Monitoring |
| | the ravulizumab Phase 2 and 3 | of immunogenicity by collection of samples |
| | studies had treatment-emergent | for assessment throughout the duration of |
| | positive ADA results. | the study. |
| | | Monitoring for infusion-related reactions will |
| | | be conducted as part of routine safety |
| | | assessments for this study. |
| | | Provisions are made in the protocol for the |
| | | clinical management of hypersensitivity in |
| | | the clinic. |
| | | Stop medication in the event of any |
| | | suspected severe AEs like immediate or |
| | | delayed hypersensitivity or anaphylaxis. |
| | | In case of a suspected SAE of |
| | | hypersensitivity or anaphylaxis, additional |
| | | ADA samples may be collected during or in |
| | | proximity of the event. |
| Pregnancy | No studies of ravulizumab have | Pregnant or nursing female participants will |
| exposure/lactation | been conducted in pregnant or | be excluded from the clinical study. |
| | breastfeeding women. There are | Participants enrolled in the study, and their |
| | no data available on excretion of | spouses/partners, must use a highly |
| | ravulizumab in breast milk. | effective or acceptable method of |
| | | contraception during treatment and for a |
| | | period of 8 months following the final dose |
| | | of ravulizumab. Breastfeeding must be |
| | | discontinued during treatment and for up to |
| | | 8 months after treatment with ravulizumab |

Abbreviations:
AE = adverse event;
ADA = antidrug antibody;
aHUS = atypical hemolytic uremic syndrome;
C5 = complement component 5;
IST = immunosuppressive therapy;
PNH = paroxysmal nocturnal hemoglobinuria;
SAE = Serious adverse event

The ravulizumab OBDS is considered an investigational drug-device combination product and as such bears some potential risks resulting from device deficiencies or use error. Risk management activities (risk control and mitigation measures) have been conducted per IS014971. Specific risk mitigation measures available to support the safe use of ravulizumab in participants in this study are described in Table 4.

TABLE 4
Important Potential Adverse Device Effects Associated with Subcutaneous
Delivery Via Ravulizumab OBDS and Mitigation Strategies

| Potential Risks | | |
|---|---|---|
| of Clinical | Summary of Data/Rationale for | |
| Significance | Risk | Mitigation Strategy |
| Injection site | Injection site reactions include the | Rotation of injection sites, hypoallergenic |
| reaction | following injection associated terms | adhesives, skin care |
| | during or within 24 hours post SC | Increased awareness of healthcare |
| | injection: injection reactions (not | professionals and participants about the |
| | otherwise specified), erythema, | potential risk of injection site reactions. |
| | rash, swelling, pruritus, ecchymosis, | Monitoring for signs and symptoms of |
| | pain, hematoma, induration, bruise, | injection site reactions will be conducted as |
| | urticaria, and inflammation | part of the safety assessments for this |
| | | study. |
| Allergy to acrylic | The OBI uses acrylic adhesive. For | Premedication can be considered, and |
| adhesives | patients with a known allergy to | supportive measures must be instituted if |
| | acrylic adhesive, use of this product | signs of allergy appear. |
| | may result in an allergic reaction | |

Abbreviations:
OBDS = on-body delivery system;
OBI = on-body injector;
SC = subcutaneous

Details of the anticipated ADEs, contraindications, and warnings for the Ravulizumab OBDS are provided in the Ravulizumab OBDS Device Investigator's Brochure.

### 2.5.1.1. Coronavirus Disease 2019

The COVID-19 pandemic is active in many countries at the time of this protocol amendment. Given this unique circumstance, specific consideration has been given to the risks and benefits of the study as they relate to COVID-19 and vaccination against the disease.

### 2.5.2. Benefit Assessment

The main objective of effective pediatric PNH and aHUS treatment with targeted therapy is to provide immediate, complete, and sustained inhibition of terminal complement activity to block hemolysis and prevent thrombosis in PNH and prevent thrombotic microangiopathy (TMA) in patients with aHUS. Given that PNH and aHUS are chronic diseases across all age categories, ravulizumab SC is expected to provide substantial therapeutic benefit in pediatric patients who are currently not receiving complement inhibitor therapy or are receiving eculizumab.

Self-administered SC treatment [(i.e., for the purposes of the study “self-administration” refers to study intervention administration by caregivers (for participants 2 to <18 years of age) or by participants (only for participants ≥12 years of age)] for patients with PNH or aHUS offers the potential for increased independence over treatment by IV infusion and may reduce the burdens and risks associated with chronic IV treatment regimens while increasing patient adherence.

### 2.5.3. Overall Benefit: Risk Conclusion

Considering the measures taken to minimize risk to participants in this study, the potential risks identified in association with ravulizumab SC are justified by the anticipated benefits that may be afforded to pediatric participants with PNH or aHUS.

Objectives and Estimands and/or Endpoints

TABLE 5
Mapping Objectives to Estimands/Endpoints

| | |
|---|---|
| Objectives | Estimands and/or Endpoints |

Primary (Both Cohorts)

| | |
|---|---|
| To characterize the PK and PD of | Primary PK Estimand: |
| treatment with ravulizumab SC in | Treatment: Ravulizumab SC |
| pediatric participants with PNH or | Population: Pediatric participants with PNH or aHUS who |
| aHUS | are included in the PK Analysis Set |
| | Variables: Ravulizumab concentrations at Day 1 postdose, |
| | Day 15 predose, Day 15 postdose, and Day 71 predose |
| | Intercurrent events (IEs): |
| | IE1: Discontinuation of study intervention prior to the |
| | end of the Primary Evaluation Period (Day 71) |
| | IE2: Initiation of specific disallowed medication or |
| | therapy prior to the end of the Primary Evaluation Period |
| | (Day 71) (Section 4.12.2) |
| | Data collected for participants after IE1 or IE2 will not be |
| | used for the primary PK analysis. |
| | Summary measures: Summary statistics of ravulizumab |
| | concentrations at Day 1 postdose, Day 15 predose, Day 15 |
| | postdose, and Day 71 predose |
| | Primary PD Estimand: |
| | Treatment: Ravulizumab SC |
| | Population: Pediatric participants with PNH or aHUS who |
| | are included in the PD Analysis Set |
| | Variables: Free C5 concentrations at Day 1 postdose, Day |
| | 15 predose, Day 15 postdose, and Day 71 predose |
| | Intercurrent events (IEs): |
| | IE1: Discontinuation of study intervention prior to the |
| | end of the Primary Evaluation Period (Day 71) |
| | IE2: Initiation of specific disallowed medication or |
| | therapy prior to the end of the Primary Evaluation Period |
| | (Day 71) (Section 4.12.2) |
| | Data collected for participants after IE1 or IE2 will not be used |
| | for the primary PD analysis. |
| | Summary measures: Summary statistics of serum free C5 |
| | concentrations at Day 1 postdose, Day 15 predose, Day 15 |
| | postdose, and Day 71 predose |

Secondary Endpoints
PK and PD (Both Cohorts)

| | |
|---|---|
| To characterize the PK and PD of | Serum ravulizumab concentrations over time through Week 52 |
| treatment with ravulizumab SC in | Absolute values, change from baseline, and percentage |
| pediatric participants with PNH or | change from baseline for free serum C5 concentrations over |
| aHUS | time through Week 52 |

Efficacy (PNH Cohort Only)

| | |
|---|---|
| To assess the efficacy of treatment | Percentage change from baseline in LDH to Week 10 and |
| with ravulizumab SC in pediatric | Week 52 |
| participants with PNH | Incidence of BTH through Week 10 and Week 52 |
| | Achievement of transfusion avoidance through Week 10 and |
| | Week 52 |
| | Achievement of stabilized hemoglobin through Week 10 and |
| | Week 52 |
| | Change from baseline in PNH RBC clone size at Week 52 |

Efficacy (aHUS Cohort Only)

| | |
|---|---|
| To assess the efficacy of treatment | Dialysis requirement status through Week 10 and Week 52 |
| with ravulizumab SC in pediatric | Observed value and change from baseline in eGFR through |
| participants with aHUS | Week 10 and Week 52 |
| | Observed value and change from baseline in serum creatinine |
| | through Week 10 and Week 52 |
| | Observed value and change from baseline in hematologic |
| | parameters through Week 10 and Week 52: |
| | Platelets |
| | LDH |
| | Hemoglobin |

Health-related QoL (Both Cohorts)

| | |
|---|---|
| To assess the effect of treatment | Change from baseline in patient-reported fatigue, as measured |
| with ravulizumab SC on fatigue and | by Pediatric FACIT-Fatigue (participants ≥8 years of age) to |
| QoL based on patient-reported and | Week 10 and Week 52 |
| caregiver-reported outcomes in | Change from baseline in PedsQL 4.0 Generic Core Scale to |
| pediatric participants with PNH or | Week 10 and Week 52 |
| aHUS | |

Safety (Both Cohorts)

| | |
|---|---|
| To evaluate safety of | Incidence of AEs and SAEs |
| ravulizumab SC in pediatric | Incidence of ADEs and serious ADEs in participants treated |
| participants with PNH or aHUS | with ravulizumab SC via OBI |

Device Performance (Both Cohorts)

| | |
|---|---|
| To assess performance of | Reported outcome of attempted full-dose administration via OBI |
| ravulizumab OBDS in pediatric | Reported device deficiencies/complaints and associated device |
| participants with PNH or aHUS | investigations |

Immunogenicity (Both Cohorts)

| | |
|---|---|
| To assess immunogenicity of | ADA incidence, response categories and titer in study |
| ravulizumab SC in pediatric | participants treated with ravulizumab SC for the duration of the |
| participants with PNH or aHUS | study |

Abbreviations:
ADA = antidrug antibody;
ADE = adverse device effect;
AE = adverse event;
aHUS = atypical hemolytic uremic syndrome;
BTH = breakthrough hemolysis;
C5 = complement component 5;
eGFR = estimated glomerular filtration rate;
FACIT-Fatigue = Functional Assessment of Chronic Illness Therapy-Fatigue;
IE = intercurrent event;
LDH = lactate dehydrogenase;
OBDS = on-body delivery system;
OBI = on-body injector;
PD = pharmacodynamic;
PedsQL = Pediatric Quality of Life Inventory;
PK = pharmacokinetic;
PNH = paroxysmal nocturnal hemoglobinuria;
QoL = quality of life;
RBC = red blood cells;
SC = subcutaneous;
SAE = serious adverse event

### 2.6. Definitions for Endpoints

### 2.6.1. Breakthrough Hemolysis (PNH Cohort Only)

Breakthrough hemolysis is defined as at least 1 new or worsening symptom or sign of intravascular hemolysis (fatigue, hemoglobinuria, abdominal pain, shortness of breath [dyspnea], anemia [hemoglobin <10 g/dL], major adverse vascular event [MAVE, including thrombosis], dysphagia, or erectile dysfunction) in the presence of elevated lactate dehydrogenase (LDH) as follows:
For complement inhibitor treatment-naïve participants: elevated LDH ≥2× upper limit of normal (ULN) after prior LDH reduction to <1.5×ULN on therapy
For eculizumab or ravulizumab-experienced participants: elevated LDH ≥2×ULN

### 2.6.2. Transfusion Avoidance (PNH Cohort Only)

Transfusion avoidance is defined as remaining transfusion-free and not requiring a transfusion from baseline to the end of the period of interest.

### 2.6.3. Stabilized Hemoglobin (PNH Cohort Only)

Stabilized hemoglobin is defined as the avoidance of a ≥2 g/dL decrease in hemoglobin level in the absence of transfusion from baseline to the end of the period of interest.

### 2.7. Co-Primary Estimands

The primary objective of this study is to characterize the PK and PD of ravulizumab SC in pediatric participants (2 to <18 years of age) with PNH or aHUS.

The co-primary estimands corresponding to the primary PK/PD objective are defined as follows:
The primary PK estimand is defined as summary statistics of ravulizumab concentrations at Day 1 postdose, Day 15 predose, Day 15 postdose, and Day 71 predose for pediatric participants with PNH or aHUS receiving treatment with ravulizumab SC who are included in the PK Analysis Set.
Any data collected after discontinuation of study intervention prior to the end of the Primary Evaluation Period or after initiation of specific prohibited medication or therapy (Section 4.12.2) during the Primary Evaluation Period will not be used in the primary PK analysis. This represents a “while on treatment” strategy.
The primary PD estimand is defined as summary statistics of free serum C5 concentrations at Day 1 postdose, Day 15 predose, Day 15 postdose, and Day 71 predose for pediatric participants with PNH or aHUS receiving treatment with ravulizumab SC who are included in the PD Analysis Set.
Any data collected after discontinuation of study discontinuation prior to the end of the Primary Evaluation Period or after initiation of specific prohibited medication or therapy (Section 4.12.2) during the Primary Evaluation Period will not be used in the primary PD analysis. This represents a “while on treatment” strategy.

The co-primary endpoints are supported by results from the ravulizumab IV programs in patients with PNH and aHUS. Evaluation of ravulizumab PK is considered to be the most appropriate measure for evaluating the suitability of the ravulizumab SC dosing regimen in pediatric patients. Evaluation of serum ravulizumab concentrations ensures that serum ravulizumab concentrations following SC administration are above the PK target concentration of 175 μg/mL used for setting the ravulizumab IV dose regimen and expected to result in complete terminal complement inhibition.

Additionally, evaluation of serum free C5 concentrations will further ensure that the ravulizumab SC dosing regimen achieves the established ravulizumab dosing goal of immediate and complete terminal complement inhibition, defined as serum free C5<0.5 ug/mL, sustained throughout treatment, which is expected to translate into clinical efficacy in patients with PNH or aHUS.

### 2.8. Secondary Estimands

### 2.9. Tertiary/Exploratory Estimands

### 3. Study Design

### 3.1. Overall Design

This is a Phase 3, open-label, multicenter study to evaluate the PK and PD of ravulizumab SC in pediatric participants (2 to <18 years of age) with documented PNH or aHUS who are either complement inhibitor treatment-naïve or have previously received complement inhibitor treatment. Participants who have previously received complement inhibitor treatment must be clinically stable and have been previously treated with eculizumab or ravulizumab for at least 90 days prior to Screening.

The study consists of a Screening Period (up to 4 weeks for complement inhibitor treatment-naïve, eculizumab-experienced participants, or ravulizumab-experienced participants weighing <20 kg, and up to 8 weeks [4 weeks for participants <20 kg] for ravulizumab-experienced participants), a 10-week Primary Evaluation Period, and an Extension Period (up to 42 weeks). All participants will be followed for safety for 8 weeks after the last dose of study intervention. Thus, the total treatment duration is up to 1 year (52 weeks) and the total study duration is up to 59 weeks (note: last dose of study intervention is on Week 51).

Approximately 22 participants will be enrolled in order to obtain at least 20 pediatric participants (2 to <18 years of age) who are evaluable for the PK/PD primary analyses will be enrolled and treated. The minimum number of participants for each age category is as follows:
2 to <6 years: at least 3 participants
6 to <12 years: at least 7 participants
12 to <18 years: at least 10 participants

All participants will be screened for eligibility during the Screening Period.

### Complement Inhibitor Treatment-Naïve Participants or Eculizumab-Experienced Participants

Complement inhibitor treatment-naïve or eculizumab-experienced participants will be screened for eligibility during the Screening Period for up to 4 weeks (28 days). Participants will receive a weight-based ravulizumab IV loading dose on Day 1 (FIG. 1). For eculizumab-experienced participants, Day 1 of study intervention will occur 2 weeks (14±2 days) from the participant's last eculizumab infusion.

During the Primary Evaluation Period, participants will receive weight-based ravulizumab SC maintenance dosing on Day 15 and qw thereafter.

### Ravulizumab-Experienced Participants

Participants who are ravulizumab-experienced will be screened for eligibility during the Screening Period for up to 8 weeks (56 days) for participants weighing ≥20 kg and up to 4 weeks (28 days) for participants weighing <20 kg). For ravulizumab-experienced participants, Day 1 of study intervention will occur 8 weeks (56±5 days) from the participant's last ravulizumab infusion for participants weighing ≥20 kg or 4 weeks (28±2 days) from the participant's last ravulizumab infusion for participants weighing <20 kg.

During the Primary Evaluation Period, ravulizumab-experienced participants will receive a weight-based ravulizumab SC maintenance dose on Day 1 (FIG. 2), followed by weight-based maintenance doses of ravulizumab SC qw thereafter.

### Extension Period (All Participants)

For all participants, Day 71 is the end of the Primary Evaluation Period and the beginning of the Extension Period. All Day 71 assessments completed prior to dosing are considered part of the Primary Evaluation Period. Dosing on Day 71 is the start of the Extension Period.

During the Extension Period, all participants will continue to receive weight-based maintenance doses of ravulizumab SC on Day 71 and qw thereafter through the end of the Extension Period.

If a participant discontinues study intervention, the participant will be withdrawn from the study and encouraged to complete the ED Visit as soon as possible.

A Safety Follow-up Visit will be conducted via phone/video call approximately 8 weeks (56±7 days) after the last dose of ravulizumab (SC or IV) to collect information on concomitant medications, nonpharmacologic therapies and procedures, AEs, and ADEs.

### 3.2. Scientific Rationale for Study Design

Participants receiving ravulizumab SC are expected to maintain ravulizumab C_{trough} concentrations above the PK target concentration needed to achieve immediate and complete terminal complement inhibition. The PK assessment is planned on Day 71 (at the time of the C_{trough} prior to dosing). The Extension Period of up to 42 weeks of treatment with ravulizumab will enable monitoring of the long-term safety and efficacy of ravulizumab treatment for pediatric patients with PNH and aHUS.

A single-arm design is deemed appropriate to investigate the PK, PD, efficacy, and safety of ravulizumab SC in a pediatric population. The rarity of PNH or aHUS in the pediatric population precludes feasibility of a study with a larger sample size. Twenty patients are considered sufficient to adequately describe PK/PD in this population.

The efficacy parameters in Study ALXN1210-PED-316 are fully aligned to those established in the Phase 3 studies in adult patients with PNH or aHUS, which is justified by the similar disease pathophysiology and anticipated clinical response to ravulizumab treatment in children and adolescents.

The availability of treatment with ravulizumab SC qw would provide an additional treatment option for patients, which may reduce the burdens associated with chronic treatment with eculizumab IV administered once every 2 weeks, ravulizumab IV administered once every 4 weeks for patients <20 kg, or ravulizumab IV administered once every 8 weeks for patients ≥20 kg, by providing the patients with a more convenient method of dose administration (self-administration of ravulizumab SC via an OBI or a syringe in the home setting).

Ravulizumab SC administered via the OBI provides patients ≥20 kg and physicians with an option for less frequent clinic visits for dose administration, which offers the potential for improved quality of life (QoL) through fewer missed days of work or school and improved accessibility.

### 3.3. Justification for Dose

The ravulizumab SC dosing goal in pediatric patients with PNH or aHUS remains unchanged from all other ravulizumab IV and SC programs, namely achieving immediate and complete terminal complement inhibition (defined as serum free C5 concentrations <0.5 ug/mL), sustained throughout treatment in all patients. As supported in prior ravulizumab development programs, maintaining ravulizumab serum concentrations >175 μg/mL achieves this goal.

Using prior ravulizumab population-PK models developed using PK data from ravulizumab IV studies (adult and pediatric patients with PNH or aHUS) or SC studies (adult patients with PNH), dose simulations were performed to propose weight-based ravulizumab SC regimens predicted to achieve an immediate, complete, and sustained inhibition of terminal complement activity in pediatric patients with PNH or aHUS. The use of dose simulations to characterize exposure-response relationships in support of Phase 3 dose selection is well established (FDA Guidance for Industry: Exposure-Response Relationships—Study Design, Data Analysis, and Regulatory Applications) and is a similar approach as has been used to inform all approved ravulizumab dose regimen.

The weight-based dosing regimen (detailed in Section 4.6) is expected to maintain serum drug concentrations above the target concentration needed for complete inhibition of terminal complement in all pediatric participants weighing ≥10 kg from the start of the treatment. The ravulizumab IV formulation has been shown to be well tolerated when administered SC via syringe at a dose of 400 mg to healthy subjects in Studies ALXN1210-HV-105 and ALXN1210-SC-101. To confirm or adjust the SC dosing regimen in pediatric participants, an interim analysis will be conducted as outlined in Section 5.5.

### 3.4. Justification for Device Selected

Ravulizumab SC is intended to be administered via SC injection for maintenance dosing in the clinic or home setting. The ravulizumab OBDS kit consists of ravulizumab SC in a sterile, single-use, prefilled cartridge and is co-packaged with a single-use OBI. Each OBDS delivers a fixed dose of 245 mg ravulizumab. Since participants weighing <20 kg require a weekly dose of 150 mg ravulizumab, which is less than the full dose (245 mg) of the ravulizumab OBDS, this patient population will not use the OBDS and will receive ravulizumab SC via syringe.

The drivers for the SC device selection were dose volume capability, demonstrated reliability, and patient-centered usability requirements including ease of use with minimal steps, minimal discomfort, hidden needle, as well as the ability to move about and perform moderate physical activities during the administration such as walking, reaching, and bending. The West SMARTDOSE® Gen I 3.5 mL device platform meets these criteria.

The ravulizumab OBDS is a disposable, single-use drug-device combination product that is configured to deliver a fixed dose of ravulizumab via SC injection in approximately 10 minutes. The device is designed to be easy to use, with a single button to initiate dosing, visual and auditory cues of the device readiness and dose completion, and a safety latch to prevent needle stick injury. The small gauge needle in the device and route of administration may carry a reduced risk of systemic infection and other complications compared with IV infusions.

### 3.5. Remote Visit Options

To ensure participant safety and treatment continuity in times of emergency (e.g., COVID-19 pandemic), the following will apply where participants are not able to reach the study sites, and until participants are able to resume study visits at the site.

Remote visit options may be performed at the Investigator's discretion and oversight, in accordance with the local regulations, and conducted by a qualified medical professional. Remote visit options may include visits conducted at the participant's home, an alternative qualified healthcare facility, or virtually via phone/video call. All assessments for the study visit day will be conducted according to the Schedule of Activities (Section 1.1). Information regarding concomitant medications, nonpharmacologic therapies and procedures (including background therapies), disease-related signs or symptomatology, AEs, and ADEs must be sent to the Investigator's site for evaluation on the day of the remote visit. In case of any signs or symptoms indicating a serious adverse event (SAE), the participant will need to be evaluated at the study site.

### 3.6. End of Study Definition

A participant is considered to have completed the study if they have completed all periods of the study including the last scheduled procedure shown in the Schedule of Activities (Section 1.1).

The end of the study is defined as the date the last participant completes their last visit (including follow-up).

### 4. Study Population

Prospective approval of protocol deviations to recruitment and enrollment criteria, also known as protocol waivers or exemptions, is not permitted.

### 4.1. Inclusion Criteria

Participants are eligible to be included in the study only if all of the following criteria apply:

### 4.1.1. Common Inclusion Criteria for Both Cohorts

### Age

1. Must be 2 to <18 years of age at the time of informed consent

### Sex

2. Male or female
3. Female participants of childbearing potential and male participants must be willing to follow protocol-specified contraception guidance

### Weight

4. Body weight ≥10 kg at Screening

### Vaccinations and Antibiotics

5. To reduce the risk of meningococcal infection (Nmeningitidis), all participants must be vaccinated against meningococcal infection from serogroups A, C, Y, W135, and B within 3 years prior to, or at least 2 weeks prior to Day 1, according to national/local guidelines. Participants who do not meet this requirement must be vaccinated against meningococcal infection according to national/local guidelines and receive prophylactic antibiotics for at least 2 weeks after meningococcal vaccination if Day 1 occurs <2 weeks after initial vaccination.
6. Must have received vaccination for Streptococcus pneumoniae according to national and local vaccination schedule guidelines
7. Must have received vaccination for Haemophilus influenzae type b according to national and local vaccination schedule guidelines Prior/Concomitant Therapy
8. Complement inhibitor-experienced participants must have been treated with eculizumab or ravulizumab according to the labeled dosing recommendation for at least 90 days prior to Screening with no missed doses within 2 months prior to study entry and no more than 2 doses outside of the visit window.

### Informed Consent

9. Participant's legal guardian/legally authorized representative must be capable of giving written informed consent and the participant must be capable of giving written informed assent (if applicable as determined by the central or local institutional review board [IRB]/independent ethics committee [lEC]) which includes compliance with the requirements and restrictions listed in the informed consent form (ICF) and in this protocol.

### 4.1.2. Inclusion Criteria Specific for PNH Cohort

### Disease Characteristics

10. Documented diagnosis of PNH confirmed by high-sensitivity flow cytometry evaluation (Borowitz et al., Cytometry Part B Clinical Cytometry. 78(4):211-230. 2010) of red blood cells (RBCs) and white blood cells (WBCs), with granulocyte or monocyte clone size of ≤5%
11. Complement inhibitor treatment-naïve participants must have the presence of 1 or more of the following PNH-related signs or symptoms within 3 months of Screening: fatigue, hemoglobinuria, abdominal pain, shortness of breath (dyspnea), anemia, history of a MAVE (including thrombosis), dysphagia, or erectile dysfunction; or history of packed red blood cell transfusion due to PNH.
12. LDH values at Screening as follows:
   a. Complement inhibitor treatment-naïve participants must have LDH ≥1.5×ULN analyzed by the central laboratory
   b. Eculizumab- or ravulizumab-experienced participants must have LDH ≥1.5×ULN (sample must be obtained within 1 day prior to the scheduled eculizumab/ravulizumab dosing day [i.e., at trough eculizumab/ravulizumab level] and analyzed by the central laboratory)
      4.1.3. Inclusion Criteria Specific for aHUS Cohort

### Disease Characteristics

13. Complement inhibitor treatment-naïve participants must have evidence of TMA, including thrombocytopenia, evidence of hemolysis, and kidney injury, based on the following laboratory findings:
   a. Platelet count <150000/μL during the Screening Period or within 28 days prior to the start of the Screening Period, and
   b. LDH ≥1.5×ULN for age and sex during the Screening Period or within 28 days prior to the start of the Screening Period, and
   c. Hemoglobin ≤lower limit of normal (LLN) for age and sex during the Screening Period or within 28 days prior to the start of the Screening Period, and
   d. Serum creatinine level ≥97.5th percentile for age at Screening (participants who require dialysis for acute kidney injury are also eligible regardless of serum creatinine level)
14. Eculizumab- or ravulizumab-experienced participants must have confirmed diagnosis of aHUS including all of the following laboratory findings documented by local laboratories at the time of the TMA event:
   a. Increase in LDH >ULN, and
   b. Increase in serum creatinine >ULN, and
   c. Decrease in platelets <LLN
15. Eculizumab- or ravulizumab-experienced participants must have clinical evidence of response to eculizumab or ravulizumab indicated by stable TMA parameters (via central laboratory results) at Screening, including:
   a. LDH <1.5×ULN, and
   b. Platelet count ≥150000/μL, and
   c. Estimated glomerular filtration rate (eGFR) >30 mL/min/1.73 m² using the Schwartz formula
16. Among participants with a kidney transplant:
   a. Known history of aHUS prior to current kidney transplant, or
   b. No known history of aHUS, and persistent evidence of TMA at least 4 days after modifying the immunosuppressive regimen (e.g., suspending or reducing the dose) of calcineurin inhibitor ((CNI); e.g., cyclosporine, tacrolimus) or mammalian target of rapamycin inhibitor ((mTORi); e.g., sirolimus, everolimus)
17. Among participants with onset of TMA postpartum, persistent evidence of TMA for >3 days after the day of childbirth.

### 4.2. Exclusion Criteria

Participants are excluded from the study if any of the following criteria apply:

### 4.2.1. Common Exclusion Criteria for Both Cohorts

Participants from both cohorts will be excluded from the study if any of the below criteria apply.

### Medical Conditions

1. History of bone marrow transplantation
2. History of N meningitidis infection
3. History of unexplained infections
4. Active systemic bacterial, viral, or fungal infection within 14 days prior to study intervention administration on Day 1
5. Presence of fever≥38° C. (100.4° F.) within 7 days prior to study intervention administration on Day 1
6. Known history or positive serology of hepatitis B or C viral infection
7. Known human immunodeficiency virus (HIV) infection (known history or evidenced by HIV type 1 or type 2 (HIV-1, HIV-2) antibody
8. History of malignancy within 5 years of Screening with the exception of nonmelanoma skin cancer or carcinoma in situ of the cervix that has been treated with no evidence of recurrence
9. History of or ongoing major cardiac, pulmonary, renal, endocrine, or hepatic disease (e.g., active hepatitis) that, in the opinion of the Investigator or Alexion, precludes the participant's participation in an investigational clinical study
10. Unstable medical conditions (e.g., myocardial ischemia, active gastrointestinal bleed, severe congestive heart failure, anticipated need for major surgery within 6 months of Day 1, coexisting chronic anemia unrelated to PNH) that would make participants unlikely to tolerate the requirements of the protocol
11. Known medical or psychological condition(s) or risk factor that, in the opinion of the Investigator, might interfere with the participant's full participation in the study, pose any additional risk for the participant, or confound the assessment of the participant or outcome of the study
12. Known or suspected history of drug or alcohol abuse or dependence within 1 year prior to the start of the Screening Period
13. History of hypersensitivity reactions to commonly used antibacterial agents, including beta-lactams, penicillin, aminopenicillins, fluoroquinolones (specifically including ciprofloxacin), cephalosporins, and carbapenems, which in the opinion of the Investigator would make it difficult to properly provide either empiric antibiotic therapy or treat an active infection

### Prior/Concomitant Therapy

14. Concomitant use of anticoagulants is prohibited if not on a stable regimen for at least 2 weeks prior to study entry

### Prior/Concurrent Clinical Study Experience

15. Participation in another experimental therapy or investigational device study within 4 weeks before initiation of study intervention on Day 1 in this study or within 5 half-lives of that investigational product, whichever is greater (except for participation in observational studies [e.g., PNH Registry])
16. Received any other experimental C5 antagonist at any time Other Exclusions
17. Pregnant, breastfeeding, or intending to conceive during the course of the study
18. Inability for participant/caregiver to complete the requirements for SC self-administration
19. Inability to travel to the clinic for specified visits during the Primary Evaluation Period or fulfil the logistic requirements of study intervention administration

### 4.2.2. Exclusion Criteria Specific for PNH Cohort

### Medical Conditions

20. More than 1 LDH value >2×ULN within the 3 months prior to study entry (eculizumab-experienced participants or ravulizumab-experienced participants only)
21. MAVE in the 6 months prior to study entry (eculizumab-experienced participants or ravulizumab-experienced participants only)
22. Platelet count <30,000/mm³ (30×10⁹/L) at Screening
23. Absolute neutrophil count <500/μL (0.5×10⁹/L) at Screening
   4.2.3. Exclusion Criteria Specific for aHUS Cohort

### Disease Characteristics

24. Hemolytic uremic syndrome related to known genetic defects of cobalamin C metabolism
25. Identified drug exposure-related HUS
26. Any known abnormal TMA parameters within 90 days prior to screening (i.e., LDH ≥1.5×ULN, or platelet count <150,000/μL, or eGFR 30 mL/min/1.73 m² using the Schwartz formula) (eculizumab-experienced participants or ravulizumab-experienced participants only)

### Medical Conditions

27. Known familial or acquired ADAMTS13 (“a disintegrin and metalloproteinase with a thrombospondin type 1 motif, member 13”) deficiency (activity <5%)
28. Known Shiga toxin-related hemolytic uremic syndrome (ST-HUS) as demonstrated by a positive test for Shiga toxin or culture of Shiga toxin producing bacteria
29. Positive direct Coombs test which in the judgment of the Investigator is indicative of a clinically significant immune-mediated hemolysis not due to TMA
30. Participants with a confirmed diagnosis of ongoing sepsis defined as positive blood cultures within 7 days prior to the start of screening and untreated with antibiotics
31. Presence or suspicion of active and untreated systemic bacterial infection that, in the opinion of the Investigator, confounded an accurate diagnosis of aHUS or impeded the ability to manage the aHUS disease
32. Heart, lung, small bowel, pancreas, or liver transplant
33. Among participants with a kidney transplant, acute kidney dysfunction within 4 weeks of transplant consistent with the diagnosis of acute cell-mediated or antibody-mediated rejection according to Banff 2013 criteria
34. Among participants without a kidney transplant, history of kidney disease other than aHUS, such as:
   a. Known kidney biopsy finding suggestive of underlying disease other than aHUS
   b. Known kidney ultrasound finding consistent with an alternative diagnosis to aHUS (e.g., small kidneys for age)
   c. Known family history and/or genetic diagnosis of noncomplement-mediated genetic renal disease (e.g., focal segmental glomerulosclerosis)
35. Known systemic sclerosis (scleroderma), systemic lupus erythematosus or antiphospholipid antibody positivity or syndrome Prior/Concomitant Therapy
36. Chronic dialysis (defined as dialysis on a regular basis as renal replacement therapy for end-stage kidney disease)
37. Received chronic IV immunoglobulin within 8 weeks prior to the start of Screening, unless for unrelated medical condition (e.g., hypogammaglobinemia); or chronic rituximab therapy within 12 weeks prior to the start of Screening
38. Received other immunosuppressive therapies such as steroids, mTORi (e.g., sirolimus, everolimus), CNI (e.g., cyclosporine or tacrolimus) unless:
   a. Part of an established post-transplant antirejection regimen, or
   b. Participant had confirmed anti-complement factor antibodies requiring immunosuppressive therapy, or
   c. Steroids were being used for a condition other than aHUS (e.g., asthma)
39. Use of tranexamic acid within 7 days prior to Screening
40. For complement inhibitor treatment-naïve participants, received plasma exchange/plasma infusion, for 28 days or longer, prior to the start of the Screening Period for the current TMA

For complement inhibitor treatment-naïve participants with aHUS, samples collected at Screening may be tested at either a local or central laboratory. If a local laboratory is used to define eligibility, additional samples will be collected during the Screening Period for platelet count, LDH, hemoglobin and serum creatinine and tested at the central laboratory. All analyses in this study will be based on results from the central laboratory (unless the result is missing). If complement inhibitor treatment-naïve participants are found to not satisfy the eligibility criteria for serum creatinine (Inclusion Criterion 13d) based on central laboratory results, they must not be enrolled into the study; if the participant has received the first dose of ravulizumab (IV or SC) the participant must be withdrawn from the study and may be replaced.

For eculizumab- or ravulizumab-experienced participants with aHUS, samples collected at Screening must be tested at a central laboratory; however, historical test results will be utilized for Inclusion Criterion 14 and Exclusion Criteria 27, 28, and 29.

### 4.3. Lifestyle Considerations

### 4.4. Screen Failures

Screen failures are defined as participants who consent to participate in the clinical study but are not subsequently enrolled (i.e., treated with study intervention). A minimal set of screen failure information is required to ensure transparent reporting of screen failure participants to meet the Consolidated Standards of Reporting Trials publishing requirements and to respond to queries from regulatory authorities. Minimal information includes demography, screen failure details (e.g., failed eligibility criteria), and any AEs, including any SAEs and any related concomitant medication, occurring during the Screening Period.

Individuals who do not meet the criteria for participation in this study (screen failure) due to a reason that is expected to resolve or has resolved, may be rescreened once based on discussion and agreement between the Investigator and the Medical Monitor. Participants who are rescreened outside of the Screening window (Section 1.1) are required to sign a new ICF.

Study Intervention Study intervention is defined as any investigational intervention(s), marketed product(s), placebo, or medical device(s) intended to be administered to a study participant according to the study protocol.

For this study, the following terminology will be used:
The term “study intervention” includes “ravulizumab IV”, “ravulizumab SC via injection”, as well as “ravulizumab SC via OBI.”
The term “study drug” refers to “ravulizumab” regardless of the route of administration.
The term “study device” refers to the “OBI.”

### 4.5. Study Interventions Administered

Details regarding the study interventions are presented in Table 6.

TABLE 6
Study Interventions Administered

| Study | | Ravulizumab SC | Ravulizumab SC |
|---|---|---|---|
| Intervention | | via Syringe | via OBDS |
| Name | Ravulizumab IV | (Participants <20 kg) | (Participants ≥20 kg) |
| IMP or | IMP | IMP | IMP |
| NIMP/AxMP | | | |
| Type | Drug | Drug | Drug/device combination |
| Dosage form | Concentrated^{a} sterile, preservative-free | | Sterile, preservative-free aqueous |
| | aqueous solution (100 mg/mL) in 3-ml vials | | solution (70 mg/mL) in single-use, |
| | | | 3.5-mL prefilled cartridge |
| | | | designed for use with a |
| | | | single-use OBI |
| Route of | IV infusion | SC injection via | SC injection via the ravulizumab |
| administration | | syringe | OBI |
| Packaging and | Ravulizumab primary closure is US | | Ravulizumab primary container |
| supply | Pharmacopeia/European Pharmacopeia | | closure is Crystal Zenith ® (cyclic |
| | Type 1 borosilicate glass vial with a | | olefinic polymer) cartridge |
| | chlorobutyl rubber stopper, an aluminum | | stoppered with butyl rubber |
| | overseal, and a polypropylene flip-off cap. | | stopper and a TSA. |
| | | | The ravulizumab OBDS will be |
| | | | supplied in a carton containing |
| | | | 1 ravulizumab prefilled cartridge |
| | | | co-packaged with 1 OBI. |
| Physical | Translucent clear to yellowish color, | | Translucent clear to yellowish |
| description of | practically free from particles | | color, practically free from |
| study drug | | | particles |

| | |
|---|---|
| Manufacturer | Alexion or contracted manufacturing organization |

Abbreviations:
AxMP = auxiliary medicinal product;
IMP = investigational medicinal product;
IV = intravenous;
NIMP = noninvestigational medicinal product
OBDS = on-body delivery system;
OBI = on-body injector;
SC = subcutaneous;
TSA = telescopic screw assembly
^{a}No dilution is needed for SC administration.
Source: Ravulizumab IV Investigator Brochure; Ravulizumab OBDS Investigator Brochure All ravulizumab SC and ravulizumab IV formulations are suitable for human use and manufactured under current Good Manufacturing Practices.

### 4.5.1. Ravulizumab

Ravulizumab IV is formulated at pH 7.4 and is supplied in 3 mL vials. Each vial of ravulizumab IV contains 300 mg of ravulizumab (100 mg/mL) in 50 mM sodium phosphate, 5% sucrose, 25 mM I-arginine, 0.05% polysorbate 80, and water for injection.

For participants weighing <20 kg, the same formulation as ravulizumab IV will be used for the SC injection and is supplied in 3 mL vials.

For participants weighing ≥20 kg, ravulizumab SC will be supplied in a kit (ravulizumab OBDS, refer to Section 4.5.2) comprised of a prefilled cartridge assembly co-packaged with a single-use OBI that is configured to deliver a fixed dose of ravulizumab via SC injection in approximately 10 minutes. Ravulizumab SC is formulated at pH 7.4 and is supplied in 3.5-mL single-use prefilled cartridges. Each cartridge of ravulizumab SC contains 245 mg of ravulizumab (70 mg/mL) in 50 mM sodium phosphate, 25 mM L-arginine, 5% sucrose, 0.05% polysorbate 80, and water for injection.

### 4.5.2. Ravulizumab On-Body Delivery System

The ravulizumab OBDS, drug-device combination product, consists of a prefilled cartridge containing ravulizumab and an OBI. The OBI is a compact, sterile, single-use, electro-mechanical, wearable injection device that administers a fixed dose of ravulizumab from a prefilled cartridge assembly into an SC tissue at a fixed rate via a stainless steel 29-gauge needle. The device is a sterile, single use, surgically invasive active medical device for transient use as per definitions from the European Union Medical Device Regulation (EU MDR) 2017/745. The device contains non removable batteries and includes an adhesive patch. The device with adhesive is removed from the skin following completion of the dose.

The primary container closure (cartridge) consists of a cartridge with a chlorobutyl elastomeric septum, a chlorobutyl elastomeric piston, and a TSA that is threaded into the piston. The prefilled CZ cartridge is co-packaged with the OBI in a 2-compartment blister tray. The prefilled cartridge assembly is loaded into the device immediately prior to use by the participant. The device is designed for use only with the provided 3.5-mL prefilled cartridge.

After loading the cartridge into the device, adhering the device to the skin, and activating the device, the 3.5-mL dose (245 mg ravulizumab) will be delivered in approximately 10 minutes. Additional details on the device, including its intended use, are provided in the ravulizumab OBDS IB and the device Instruction for Use (IFU).

### 4.6. Study Intervention Dosing Regimen

For participants who are complement inhibitor treatment-naïve or eculizumab-experienced, the weight-based ravulizumab SC dosing regimen consists of a ravulizumab IV loading dose (Table 7) with maintenance dosing initiating 2 weeks after administration of the loading dose.

Ravulizumab-experienced participants do not require a ravulizumab IV loading dose and will only receive weight-based ravulizumab SC dosing during the study (Table 8).

For participants weighing ≥10 to <20 kg, one full maintenance dose of ravulizumab (150 mg) requires 1.5 mL SC injection as a single administration using a syringe.

For participants weighing ≥20 to <40 kg, one full maintenance dose of ravulizumab (245 mg) requires 1 ravulizumab OBDS (delivering 245 mg). For participants weighing ≥40 kg, one full maintenance dose of ravulizumab (490 mg) requires 2 ravulizumab OBDS (each delivering 245 mg), administered either concurrently or sequentially.

Dosages will be based on the participant's weight collected at the protocol-required body weight measurement. If the protocol-required body weight measurement is not available, the weight recorded at the previous protocol-required body weight measurement will be used.

If a participant who weighs <20 kg at the time study entry (Day 1) subsequently has a body weight increase to 20 kg during a protocol-required visit during which weight is measured, the participant will transition from ravulizumab SC dosing via syringe to ravulizumab SC dosing via OBI administration for the remainder of the study.

TABLE 7
Weight-based Ravulizumab Dosing Regimen for Complement
Inhibitor Treatment-Naïve Participants
or Eculizumab-Experienced Participants

| | | | Ravulizumab SC | |
|---|---|---|---|---|
| | Ravulizumab | | Maintenance | |
| | IV | | Dosing Regimen | |
| Body Weight | Loading Dose | | (Day 15 and | |
| Range (kg) | (Day 1) | | thereafter) | Delivery Method |
| ≥10 to <20 | 600 | mg | 150 mg qw | 1.5 mL via syringe |
| ≥20 to <30 | 900 | mg | 245 mg qw | 1 ravulizumab OBDS |
| ≥30 to <40 | 1200 | mg | | kit |
| ≥40 to <60 | 2400 | mg | 490 mg qw | 2 ravulizumab OBDS |
| ≥60 | 2700 | mg | | kits |

Abbreviations:
IV = intravenous;
OBDS = on-body delivery system;
qw = every week;
SC = subcutaneous

TABLE 8
Weight-based Ravulizumab Dosing Regimen
for Ravulizumab-Experienced Participants

| | Ravulizumab SC | |
|---|---|---|
| Body Weight | Maintenance Dosing Regimen | |
| Range (kg) | (Day 1 and thereafter) | Delivery Method |
| ≥10 to <20 | 150 mg qw | 1.5 mL via syringe |
| ≥20 to <30 | 245 mg qw | 1 ravulizumab OBDS kit |
| ≥30 to <40 | | |
| ≥40 to <60 | 490 mg qw | 2 ravulizumab OBDS kits |
| ≥60 | | |

Abbreviations:
OBDS = on-body delivery system;
qw = every week;
SC = subcutaneous

### 4.7. Dose Administration Instructions

### 4.7.1. Ravulizumab IV—In-Clinic Administration

For participants who are complement inhibitor treatment-naïve or eculizumab-experienced, ravulizumab IV dosing on Day 1 will be administered in the clinic by a qualified study site personnel. The ravulizumab loading dose for each participant will be based on body weight at Screening.

### 4.7.2. Ravulizumab SC—In-Clinic or At-Home Administration

On the first day of ravulizumab SC dosing, a qualified member of the site study team will provide initial (and ongoing as appropriate) training to the participant/caregiver on how to properly self-administer ravulizumab SC (i.e., administration by caregivers for participants 2 to <18 years of age or participants ≥12 years of age).

Training will include the use of the ravulizumab OBDS (drug-device combination) for participants ≥20 kg or the procedure for injection via syringe for participants <20 kg. Following the completion of required training, caregivers/participants may self-administer the subsequent ravulizumab SC injections at home on dosing days that do not coincide with an on-site visit specified in the Schedule of Activities (Section 1.1). Caregivers/participants are expected to follow the training instructions on each dosing day to ensure appropriate administration of their ravulizumab dose. Participants <20 kg will be provided the Injection Guide for Parents/Caregivers for the injection via syringe. Participants ≥20 kg will be provided the IFU for the ravulizumab OBDS.

Once training has been completed, ravulizumab SC may be self-administered by the participant/caregiver at home. The participant/caregiver can administer ravulizumab SC at the clinic on dosing days that are not scheduled in-clinic visits. Doses that coincide with on-site study visits may be self-administered in the clinic with oversight by qualified study site personnel or administered by study site personnel.

In the event of any device malfunction resulting in no dose or a partial dose is delivered, the site or participant/caregiver must contact the Call Center to ensure that the participant receives at least 245 mg of ravulizumab SC (participants weighing ≥20 to <40 kg) or at least 490 mg of ravulizumab SC (participants weighing ≥40 kg) (see Section 4.24 for reporting device complaints).

For study visits that require PK sample collection, caregivers/participants will be instructed to abstain from administering the ravulizumab dose on the day of their study visits so that the participant can be dosed in the clinic following the protocol-required assessments. At the clinic visit, the dose of ravulizumab assigned for that day will be administered at the end, after participants have blood drawn for clinical laboratory and other evaluations as outlined in the Schedule of Activities (Section 1.1).

### 4.8. Packaging and Labeling

In embodiments, the devices and systems of the disclosure for subcutaneously administering the study intervention (e.g., ravulizumab) may be labeled according to the country's regulatory requirements.

### 4.8.1. Ravulizumab Vial Packaging

The primary packaging of ravulizumab IV consists of a 3-mL vial (Type I borosilicate glass) with a stopper and a seal. The secondary packaging consists of a single vial carton. Both primary (vial) and secondary (carton) packaging include a booklet label with relevant information.

The 3-mL vial will be used for IV loading dose for complement inhibitor treatment-naïve participants or eculizumab-experienced participants. The 3-mL vial will also be used for the SC maintenance dosing for participants weighing ≥10 kg to <20 kg.

### 4.8.2. Device Packaging

The ravulizumab OBDS, a drug-device combination product, consists of 2 parts: a prefilled cartridge containing ravulizumab and the OBI. The prefilled cartridge and device constituent parts are co-packaged in a thermoformed blister pack with a Tyvek lid over the compartment containing the device to provide a sterile barrier. The secondary packaging consists of a blank carton containing the blister pack and a booklet label with relevant instructions.

An identification trace label is attached to the Tyvek covered blister, a serial number label attached to the side of the device, and a single panel label is affixed to each cartridge.

### 4.9. Preparation/Handling/Storage/Accountability

Study intervention kits will be released to each site upon receipt of all required documentation based upon applicable regulations.

### 4.10. Measures to Minimize Bias: Randomization and Blinding

This is an open-label study. The specific intervention to be administered to a participant will be assigned by the Interactive Response Technology (IRT) system based on previous C5 treatment experience and body weight.

### 4.11. Study Intervention Compliance

Ravulizumab IV will be administered in a controlled setting under the oversight of the Investigator or designee, thereby ensuring compliance with study intervention administration.

At scheduled in-clinic visits, ravulizumab SC doses may be self-administered (participants ≥12 years of age) or administered by a caregiver (participants 2 to <18 years of age) or by trained study site personnel, thereby ensuring compliance with study intervention administration.

The role of the person performing the SC dose administration, the location of the SC dose administration (i.e., home or study site), and the site of SC administration on the participant's body will be recorded on the case report form (CRF).

The study site personnel will monitor self-administration of ravulizumab SC via phone/video calls with the participant/caregiver on scheduled at-home dosing days during the Primary Evaluation Period to ensure that the participant/caregiver is queried about study intervention dose administered and device condition.

Sites will monitor self-administration of ravulizumab SC via the participant electronic diary (e-diary) throughout the study. Participants/caregivers will be instructed to contact the Call Center if there are any issues with dose administration.

A record of the number of kits dispensed to and administered for each participant must be maintained and reconciled with the number of kits and compliance records at the study site. The date and time of each dose administered will be recorded in the source documents and in the CRF.

### 4.12. Concomitant Therapy

Concomitant medications (including any medication, vitamin, herbal preparation, or supplement) and non-drug therapies/procedures (any therapeutic intervention, such as surgery/biopsy or physical therapy) are those received on or after the date of the first dose of study intervention (Day 1), including those started before Day 1 and continued after Day 1.

Any concomitant medication deemed necessary for the participant's care during the study, or for the treatment of any AE, along with any other medications, other than those listed as disallowed medications in Section 4.12.2, may be given at the discretion of the Investigator. However, it is the responsibility of the Investigator to ensure that details regarding all medications are recorded in full in the participant's source document/medical chart and CRF (Section 4.25.1).

The Medical Monitor should be contacted if there are any questions regarding concomitant or prior therapy.

### 4.12.1. Allowed Medicine and Therapy

Any concomitant medication or non-drug therapies/procedures deemed necessary for the participant's standard of care during the study, or for the treatment of any AE, along with the allowed medications described below may be given at the discretion of the Investigator.

### 4.12.2. Disallowed Medicine and Therapy

Participants in both cohorts are prohibited from receiving any of the following medications and therapies during the entire duration of study participation:
Eculizumab or other complement inhibitors
Other investigational drug or device as part of a clinical study
Anticoagulants if not on a stable dose regimen for at least 2 weeks prior to study entry

Participants in the aHUS cohort are also prohibited from receiving any of the following medications and therapies during the Screening Period and throughout the study:
Intravenous immunoglobulin (unless for an unrelated medical need, such as hypogammaglobinemia)
Rituximab
Plasma exchange/plasma infusion
New dialysis within the first 48-hour period following the first dose of ravulizumab unless there is a compelling medical need as assessed by (1) hypervolemia unresponsive to diuretics, (2) refractory electrolyte imbalance, or (3) new-onset uremic encephalopathy. Exceptions must be approved prior to administration of dialysis on a case-by-case basis by the Medical Monitor.

For participants in the aHUS cohort, the following concomitant medications and procedures are allowed under certain circumstances and with the following restrictions:
Use of other immunosuppressive therapies (such as steroids, mTORi (e.g., sirolimus, everolimus), CNI (e.g., cyclosporine or tacrolimus)) during the study are not allowed unless: a) part of an established post-transplant antirejection regimen, or b) participant has confirmed anti-complement factor antibodies requiring immunosuppressive therapy, or c) steroids are being used for a condition other than aHUS (e.g., asthma), or d) steroids initiated empirically prior to enrollment and are being tapered as standard of care.

In the event that a participant receives a prohibited medication and/or therapy, the participant may be required to discontinue study intervention and withdraw from the study (Section 4.15).

### 4.13. Dose Modification

Dose modification of the study intervention for an individual participant is not permitted for this study.

### 4.14. Intervention after the End of the Study

After a participant completes the study or discontinues study intervention, ravulizumab will not be provided to the participants as part of this protocol (Section 1.1).

Upon completion of the last study visitor at the time of study withdrawal, participants will return to the care of their treating physician.

### Discontinuation of Study Intervention and Participant

### Discontinuation/Withdrawal

### 4.15. Discontinuation of Study Intervention

A participant or their legal guardian/legally authorized representative has the right to discontinue study intervention at any time (a pediatric participant's dissent must be respected).

Participants must be discontinued from study intervention if any of the following occur during the study:
Severe uncontrolled infection
Serious infusion or hypersensitivity reaction
Pregnancy or planned pregnancy

In addition, the study intervention may be discontinued for any of the following reasons:
AE that would, in the opinion of the Investigator, make continued participation in the study an unacceptable risk
Use of disallowed medication as defined in Section 4.12.2
Deviation(s) from the protocol
Significant noncompliance
Alexion or the Investigator deems it is necessary for the participant
Termination of the study

If study intervention is discontinued, the participant will be withdrawn from the study.

If a participant discontinues study intervention due to an AE, including SAEs, the event must be followed.

Data collected at the time of study intervention discontinuation and Safety Follow-up evaluations that need to be completed is provided in the Schedule of Activities (Section 1.1). The reason for discontinuation of study intervention will be recorded in the source documents and CRF.

### 4.16. Participant Discontinuation/Withdrawal from the Study

All efforts should be made to ensure participants are willing to comply with study participation prior to conducting the screening procedures.
The study staff must notify Alexion and their site monitor of all study withdrawals as soon as possible. The reason for participant discontinuation must be recorded in the source documents and CRF.
A participant may withdraw from the study at any time at the participant's or their legal guardian/legally authorized representative's request or may be withdrawn at any time at the discretion of the Investigator for safety, behavioral, compliance, or administrative reasons.
At the time of withdrawing from the study, the following activities should be completed:
   ED Visit should be performed as outlined in the Schedule of Activities (Section 1.1), if possible.
   A Safety Follow-up Visit (via phone/video call) will be performed 8 weeks (56 days)±7 days after the participant's last dose of study intervention to collect AEs/ADEs, concomitant medications, nonpharmacologic therapies, and procedures.
If the participant or their legal guardian/legally authorized representative withdraws consent for disclosure of future information, Alexion may retain and continue to use any data collected before such a withdrawal of consent.
If a participant is withdrawn from the study, the participant or their legal guardian/legally authorized representative may request destruction of any samples taken and not tested, and the Investigator must document this in the site study records.

### 4.17. Lost to Follow-Up

A participant will be considered lost to follow-up if the participant repeatedly fails to return for scheduled visits and is unable to be contacted by the study site.

The following actions must be taken if a participant fails to return to the clinic for a required study visit:
The site must attempt to contact the participant's legal guardian/legally authorized representative to reschedule the missed visit as soon as possible, counsel the participant's legal guardian/legally authorized representative and the participant on the importance of maintaining the assigned visit schedule and ascertain whether or not the participant wishes to and/or should continue in the study.
Before a participant is deemed lost to follow-up, the Investigator or designee must make every effort to regain contact with the participant's legal guardian/legally authorized representative (where possible, 3 telephone calls and, if necessary, a certified letter to the last known mailing address or local equivalent methods). These contact attempts must be documented in the participant's medical record.
Should the participant's legal guardian/legally authorized representative continue to be unreachable, participant will be considered lost to follow-up.

### Study Assessments and Procedures

Study procedures and their timing are summarized in the Schedule of Activities (Section 1.1). Protocol waivers or exemptions are not allowed.
Immediate safety concerns will be discussed with Alexion immediately upon occurrence or awareness to determine if the participant should continue or discontinue study intervention.
Adherence to the study design requirements, including those specified in the Schedule of Activities (Section 1.1), is essential and required for study conduct.
All screening evaluations must be completed and reviewed to confirm that potential participants meet all eligibility criteria. The Investigator will maintain a screening log to record details of all participants screened and to confirm eligibility or record reasons for screening failure, as applicable.
See Section 5.8 for the list of clinical laboratory tests.
Procedures conducted as part of the participant's routine clinical management (e.g., blood count) and obtained before signing of the ICF may be utilized for screening or baseline purposes provided the procedures met the protocol-specified criteria and were performed within the time frame defined in the Schedule of Activities (Section 1.1).
If a participant fails to return to the clinic, or is otherwise unavailable, for a scheduled visit within the acceptable visit window, the site study staff must make a reasonable attempt to contact the participant/participant's caregiver to determine the reason for missing the appointment. The participant/participant's caregiver will be advised to return to the investigational site for evaluation, if an AE is suspected to have occurred. In this event, the investigational site will make a reasonable attempt to obtain all relevant medical records, and enter relevant data in the CRF, as appropriate.
Unscheduled visits that occur outside the protocol-specified visits are permitted at the discretion of the Investigator. Results for procedures, tests, and assessments conducted during unscheduled visits will be documented on the CRF.

### 4.18. General Assessments and Procedures

### 4.18.1. Informed Consent

The Investigator, or qualified designee, must obtain a signed and dated informed consent/assent form from each participant prior to conducting any study procedures. All efforts will be made to ensure participants are willing to comply with study participation prior to conducting the screening procedures.

### 4.18.2. Inclusion/Exclusion Criteria

All inclusion (Section 4.1) and exclusion (Section 4.2) criteria must be reviewed by the Investigator or qualified designee to determine whether the participant is eligible for the study.

### 4.18.3. Demographics

Demographic parameters will be documented in the CRF, if allowed per country specific regulations.

### 4.18.4. Medical History and PNH/aHUS History

The Investigator will review the participant's history and diagnosis and document the following at the Screening Visit:
Medical history including all relevant medical/surgical history
Diagnosis of PNH confirmed by high-sensitivity flow cytometry evaluation (participants in the PNH cohort only)
aHUS medical history, including onset of first aHUS symptom and date of diagnosis (participants in the aHUS cohort only)
Medical history may be used to document ADAMTS13 and Shiga toxin status (participants in the aHUS cohort only)

Any changes to medical history occurring during the Screening Period and prior to first dose of study intervention on Day 1 will be documented.

### 4.18.5. Vaccine and Antibiotic Prophylaxis

Due to its mechanism of action, the use of ravulizumab increases a participant's susceptibility to meningococcal infection (N meningitidis). To reduce the risk of meningococcal infection, all participants must be vaccinated against meningococcal infection within the 3 years prior to and at least 2 weeks before the first dose of study intervention. Participants who have not been vaccinated prior to starting study intervention for any reason must receive appropriate prophylactic antibiotics prior to Day 1 of dosing and for at least 2 weeks after vaccination. Vaccines against serotypes A, C, Y, W135, and B, where available, are recommended. Participants must receive the complete primary vaccination series and be revaccinated if indicated according to current national vaccination guidelines. Vaccination may not be sufficient to prevent meningococcal infection.

Participants will be administered prophylactic antibiotics for meningococcal infection until at least 2 weeks after vaccination if study intervention initiation occurs <2 weeks after initial vaccination. Consideration should be given per official guidance and local practice on the appropriate use of prophylactic antibacterial agents. All participants will be monitored for early signs of meningococcal infection, evaluated immediately if infection is suspected, and treated with appropriate antibiotics, if necessary.

To increase risk awareness and promote quick disclosure of any potential signs or symptoms of infection experienced by the participants during the course of the study, participants will be provided a Participant Safety Card to carry with them at all times (Section 4.21.1).

Meningococcal vaccination within 3 years prior to the first dose of study intervention, and vaccination history for Haemophilus influenzae type b and Streptococcus pneumoniae from birth, will be recorded on the CRF.

### 4.19. Efficacy Assessments

### 4.19.1. Laboratory Assessments for Efficacy Endpoints

The following disease-related laboratory parameters will be measured during the study will be performed according to the Schedule of Activities (Section 1.1) as part of the efficacy endpoints: PNH Cohort:
LDH (If a suspected event of breakthrough hemolysis occurs, an unscheduled visit must take place at which a sample is collected for analysis of LDH by the central laboratory.)
Hemoglobin
PNH clone size

For participants in the PNH cohort, hemoglobin and LDH will be assessed based on the predetermined criteria to determine breakthrough hemolysis and stabilized hemoglobin as defined in Section 2.6.

aHUS Cohort:
Estimated glomerular filtration rate (calculated using the Schwartz formula)
Serum creatinine
Platelets
LDH
Hemoglobin

A summary of laboratory assessments and procedures for blood collection are provided in Section 5.8 and Section 5.10, respectively.

### 4.19.2. Transfusion History and Transfusion Requirement Status During the Study (PNH Cohort Only)

The number and volume of transfusions ≤1 year prior to first dose of study intervention and during the study will be documented on the CRF. The information to be collected includes date of the transfusion, number of units, volume of each blood component given; hemoglobin result, and the symptoms that triggered the transfusion.

4.19.3. Dialysis Status (aHUS Cohort Only)

Participant dialysis status ≤56 days prior to first dose of study intervention and during the study will be documented on the CRF.

### 4.20. Health-Related Quality of Life

The QoL assessments should be administered prior to performing other study procedures, whenever possible, and must be administered prior to study intervention administration.

### 4.20.1. Pediatric FACIT-Fatigue

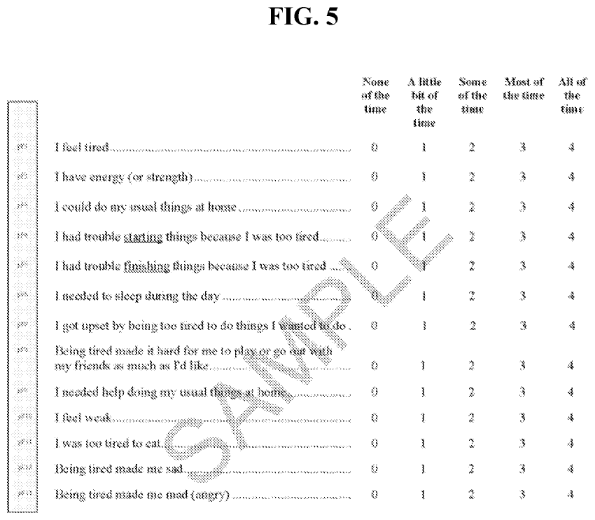

The Pediatric Functional Assessment of Chronic Illness Therapy-Fatigue (FACIT-Fatigue) scale is a 13-item questionnaire that assesses fatigue and its impact upon daily activities and function over the preceding 7 days. Each item is scored on a 5-point scale, and total scores range from 0 to 52, with higher score indicating less fatigue. The questionnaire will be self-reported by participants who were ≥8 years of age at the time of informed consent. Patients <8 years of age at the time of informed consent will not be assessed. The Pediatric FACIT-Fatigue-scale is shown in FIG. 5.

### 4.20.2. Pediatric Quality of Life Inventory

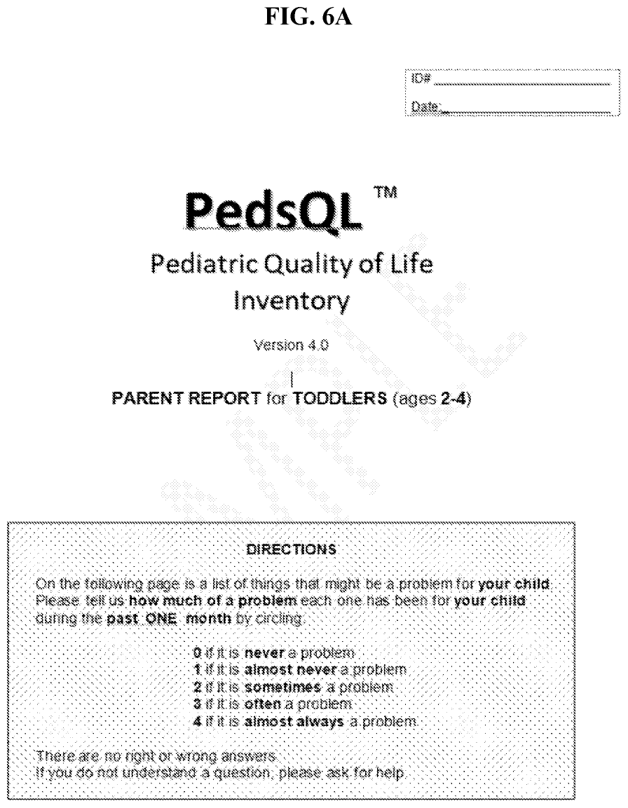

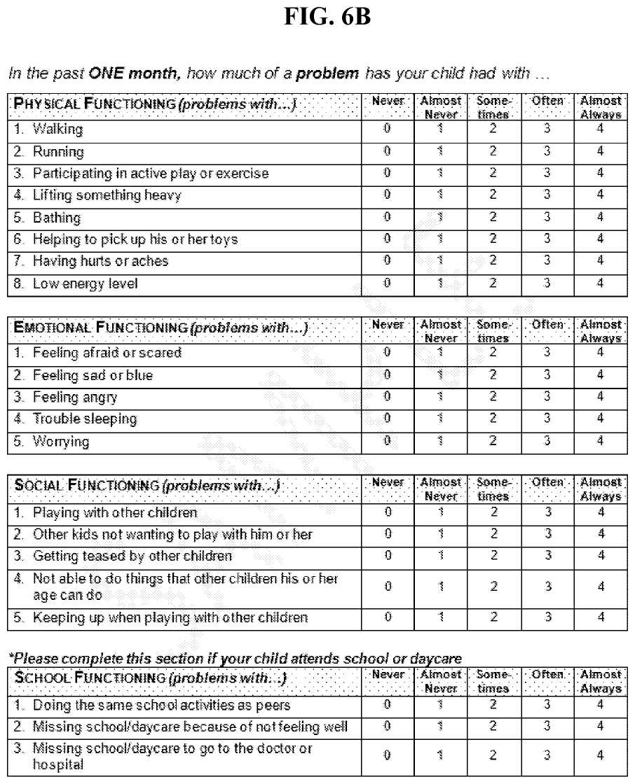

The Pediatric Quality of Life Inventory (PedsQL) 4.0 Generic Core Scales (FIGS. 6A and 6B) are multidimensional child self-report and parent proxy-report standardized instruments to measure health-related QoL in children and adolescents 2 to <18 years of age.

The test is designed for self-completion in participants aged 5 years by respondents (with 3 age-appropriate PedsQL tests available for participants 5 to 7 years of age, 8 to 12 years of age, and 13 to <18 years of age) as well as by parent proxy-report. In participants 2 to <5 years of age, the test is designed for completion by parent proxy-report only. Attempts should be made to have the same proxy complete the evaluation at each visit, whenever feasible.

The age-appropriate assessment for a participant at the time of informed consent will be used for that participant throughout the study. Change in age during the study will not constitute a participant changing the type of survey completed (e.g., for participants aged 2 years to <5 years at the time of informed consent, the PedsQL parent proxy will be used throughout the study).

### 4.21. Safety Assessments

### 4.21.1. Participant Safety Card

Before the first dose of the study intervention, a Participant Safety Card will be provided to the participant/caregiver to carry with them at all times until 8 months after the final dose of study intervention. The card is provided to increase participant/caregiver awareness of the risk of meningococcal infection and promote quick recognition and disclosure of any potential signs or symptoms of infection experienced during the course of the study and to inform participants on what actions must be taken if they are experiencing signs or symptoms of infection.

At each visit throughout the study, the study staff will review to ensure that the participant has the Participant Safety Card.

### 4.21.2. Physical Examinations

A complete physical examination will include, at a minimum, assessments of the following organs/body systems: skin, head, ears, eyes, nose, throat, chest, heart, abdomen, extremities, musculoskeletal, and neurological state.
An abbreviated physical examination consists of a body-system relevant examination based upon Investigator's (or qualified designee) judgment and participant symptoms. At least 1 body system must be checked for an abbreviated examination.
Investigators should pay special attention to clinical signs related to previous serious illnesses.
For consistency, all efforts should be made to have the physical examination performed by the same qualified study staff.
Weight and height for all participants, and head circumference for participants ≤3 years of age at time of assessment will also be measured and recorded at specified visits in the Schedule of Activities (Section 1.1).

### 4.21.3. Vital Signs

Body temperature (° C. or ° F.), respiratory rate, systolic and diastolic blood pressure (mm Hg), and heart rate (beats/minute) will be assessed.
Blood pressure and heart rate measurements will be assessed with the participant in a seated or supine position using a completely automated device. Manual techniques will be used only if an automated device is not available.
Blood pressure and heart rate measurements must be preceded by at least 5 minutes of rest for the participant in a quiet setting without distractions (e.g., television, cell phones). Ideally, the same arm for each participant should be used for measurements.
Vital signs will be collected predose at ravulizumab dosing visits.

### 4.21.4. Electrocardiograms

Single 12-lead electrocardiogram (ECGs) will be conducted locally to obtain heart rate, PR, QRS, interval between the start of the Q wave and the end of the T wave in an ECG (QT), and corrected QT (QTc) intervals. QT interval will be corrected for heart rate using Fridericia's formula (QTcF).
Participants should be supine for approximately 5 to 10 minutes before ECG collection and remain supine but awake during ECG collection.
The Investigator or designee will be responsible for reviewing the ECG to assess whether the ECG is within normal limits and determine the clinical significance of the results.

### 4.21.5. Clinical Safety Laboratory Assessments

The Investigator must review the laboratory report, document this review, and record any clinically relevant changes occurring during the study in the AE section of the CRF. The laboratory reports must be filed with the source documents. Clinically significant abnormal laboratory findings are those which are not associated with the underlying disease, unless judged by the Investigator to be more severe than expected for the participant's condition.
All laboratory tests with values considered clinically significantly abnormal during participation in the study or within 8 weeks after the final dose of study intervention will be repeated until the values return to normal or baseline or are no longer considered clinically significant by the Investigator or Medical Monitor.
   If such values do not return to normal/baseline within a period of time judged reasonable by the Investigator, the etiology should be identified, and Alexion notified.
   All protocol-required laboratory assessments, as defined in Section 5.8, must be collected in accordance with the Laboratory Manual, blood sampling volumes (Section 5.10), and the Schedule of Activities (Section 1.1).
   Laboratory assessments performed at the institution's local laboratory that require a change in participant management or are considered clinically significant by the Investigator must be recorded in the AE or SAE CRF.

### 4.21.6. Pregnancy

Pregnancy testing must be performed on all women of childbearing potential (WOCBP) at protocol-specified time points in the Schedule of Activities (Section 1.1). Pregnancy tests (urine or serum) may also be performed at any time during the study at the Investigator's discretion.
A negative pregnancy test is required for WOCBP before study intervention administration.
Pregnancy data from female participants and female spouses/partners of male participants will be collected from the first dose of study intervention and at the time points specified in the Schedule of Activities (Section 1.1). Any female participant who becomes pregnant during the study will be discontinued from the study intervention and withdrawn from the study. If a pregnancy is reported, the Investigator must immediately inform Alexion within 24 hours of awareness of the pregnancy.
For a female participant who becomes pregnant, this information will be shared with the study participant's legal guardian/legally authorized representative as required by local regulations.

### 4.22. Adverse Events, Serious Adverse Events, Adverse Device Effects, Unanticipated Serious Adverse Device Effect, and Serious Adverse Device Effects

All AEs and ADEs will be reported to the Investigator or qualified designee by the participant (or, when appropriate, by their caregiver/legal guardian/legally authorized representative) or site staff who operate the device or others who may incur any untoward medical occurrences associated with the device or use of the device.

Study site staff will instruct the participant's caregiver/legal guardian/legally authorized representative on how to report signs and symptoms (e.g., crying and pain) in the individual pediatric participant. They will be instructed to report both specific and nonspecific symptoms (including vomiting, diarrhea, sleepiness, variation in the intensity and pattern of crying, etc.). Care should be taken that the clinical presentation of AEs is not misinterpreted as the manifestation of a pre-existing condition. Moreover, symptoms that are dependent on participant communication ability (e.g., nausea, pain, mood alterations) in younger children or children with developmental delay could potentially be at risk for under- or mis-reporting.

The Investigator and any qualified designees are responsible for detecting, documenting, and recording events that meet the definition of an AE, SAE, ADE, or SADE and remain responsible for following up AEs/ADEs that are serious, considered related to the study intervention or study procedures, or that caused the participant to discontinue the study intervention (as described herein).

### 4.22.1. Time Period and Frequency for Collecting AE, SAE, ADE, and SADE Information

All AEs and SAEs will be collected from the signing of the ICF until 8 weeks after the last dose of study intervention (i.e., Safety Follow up Visit).

All ADEs and SADEs will be collected from immediately before the initial use of the ravulizumab OBDS until 8 weeks after the last dose of study intervention (i.e., Safety Follow up Visit).

All SAEs and SADEs will be recorded and reported to Alexion immediately and under no circumstance should this exceed 24 hours. The Investigator will submit any updated SAE data to Alexion within 24 hours of the date the investigational site became aware of the event.

Investigators are not obligated to actively seek AE, SAE, ADE, or SADE data after conclusion of the study participation. However, if the Investigator learns of any SAE or SADE, including a death, at any time after a participant has been discharged from the study, and they consider the event to be reasonably related to the study intervention or study participation, the Investigator must promptly notify Alexion.

### 4.22.2. Method of Detecting AEs, SAEs, ADEs and SADEs

Care will be taken not to introduce bias when detecting AEs, SAEs, ADEs, and/or SADEs. Open-ended and nonleading verbal questioning of the participant/caregiver is the preferred method to inquire about AE and ADE occurrences. Each ADE is associated with at least 1 unit (kit) of the study device. Once an ADE is collected, the unique study device (kit) serial number(s)/lot number(s) or other device identification must be immediately acquired and recorded along with the corresponding ADE.

Note: The word “kit” represents the unique number that is assigned by Alexion to the device/drug combination product and is not intended to represent a combination kit (per 21 CFR 3.2], an in vitro diagnostic device kit (per European Union In Vitro Medical Devices Regulation 2017/746 (EU IVDR 2017/746)), or a convenience kit (per 21 CFR 801.3).

### 4.22.3. Follow-up of AEs, SAEs, ADEs, and SADEs

After the initial AE/SAE report, the Investigator is required to proactively follow-up on each participant at subsequent visits/contacts. All SAEs will be followed up until resolution, stabilization, the event is otherwise explained, or the participant is lost to follow-up (as defined in Section 4.17).

### 4.22.4. Regulatory Reporting Requirements for SAEs and SADEs

Prompt notification of an SAE by the Investigator to Alexion is essential so that legal obligations and ethical responsibilities towards the safety of participants and the safety of a study intervention under clinical investigation are met.
Alexion has a legal responsibility to notify both the local regulatory authority and other regulatory agencies about the safety of a study intervention under clinical investigation. Alexion will comply with country-specific regulatory requirements relating to safety reporting to the regulatory authority, IRBs/IECs, and Investigators.
Alexion is required to submit individual suspected unexpected serious adverse reaction (SUSAR) reports in the format of MedWatch 3500 or CIOMS I Form to health authorities and Investigators as required. Forms submitted to Investigators will be blinded to treatment assignment. In limited circumstances, the blind may be broken in the case of urgent safety issues that could compromise participant safety.
An Investigator who receives an Investigator safety report describing an SAE or other specific safety information (e.g., summary or listing of SAEs) from Alexion will review and then file it along with the Investigator's Brochure and will notify the IRB/IEC, if appropriate according to local requirements.
Reporting of SADEs will be done according to regional, national, and local requirements
Under the EU CTR 536/2014, events other than SAEs (e.g., unexpected events) that may impact the benefit-risk balance should be reported.

### 4.22.5. Medication Error, Drug Abuse, and Drug Misuse

### 4.22.5.1. Timelines

If an event of medication error, drug abuse, or drug misuse occurs during the study, then the Investigator or other site personnel informs the appropriate Alexion representatives within one calendar day, i.e., immediately but no later than 24 hours of when they become aware of it.

The designated Alexion representative works with the Investigator to ensure that all relevant information is completed within one (initial fatal/life-threatening or follow-up fatal/life-threatening) or 5 (other serious initial and follow-up) calendar days if there is an SAE associated with the event of medication error, drug abuse, or misuse and within 30 days for all other events.

### 4.22.5.2. Medication Error

For the purposes of this clinical study a medication error is an unintended failure or mistake in the treatment process for a study intervention that either causes harm to the participant or has the potential to cause harm to the participant.

### 4.22.5.3. Drug Abuse

Drug abuse is the persistent or sporadic intentional, non-therapeutic excessive use of study intervention for a perceived reward or desired non-therapeutic effect.

### 4.22.5.4. Drug Misuse

Drug misuse is the intentional and inappropriate use (by a study participant) of study intervention for medicinal purposes outside of the authorized product information, or for unauthorized study intervention, outside the intended use as specified in the protocol and includes deliberate administration of the product by the wrong route.

### 4.22.6. Adverse Events of Special Interest

Meningococcal infections will be considered to be AEs of special interest.

### 4.22.7. Infusion Reactions

Local (infusion site or injection site reactions), systemic (infusion-associated/infusion-related reactions), and immune-mediated reactions (hypersensitivity) will be evaluated during the study.

### 4.23. Major Adverse Vascular Events (PNH Cohort Only)

Major adverse vascular events will be assessed as part of the planned evaluation for AEs as described in Section 4.22. A MAVE is defined as any of the following:

| | |
|---|---|
| Thrombophlebitis/deep vein thrombosis | Mesenteric/visceral vein thrombosis or infarction |
| Pulmonary embolus | Mesenteric/visceral arterial thrombosis or infarction |
| Myocardial infarction | Cerebral arterial occlusion/cerebrovascular accident |
| Transient ischemic attack | Cerebral venous occlusion |
| Unstable angina | Renal arterial thrombosis |
| Renal vein thrombosis | Gangrene (nontraumatic; nondiabetic) |
| Acute peripheral vascular occlusion | Amputation (nontraumatic; nondiabetic) |
| Hepatic/portal vein thrombosis (Budd-Chiari | Dermal thrombosis |
| syndrome) | Other (to be specified) |

The description of the MAVE including the method of diagnosis (e.g., magnetic resonance imaging, ultrasound, angiogram), date of diagnosis, and date resolved (or ongoing) will be collected on the CRF as part of the patient's medical history (prior to baseline).

### 4.24. Medical Device Deficiencies/Complaints

In order to fulfill regulatory reporting obligations worldwide, the Investigator is responsible for the detection and documentation of events meeting the definitions of medical device deficiency/complaint that occur during the study with such devices.

All device issues must be reported, regardless of whether the full volume of the drug was administered from the ravulizumab OBI.

Note: Deficiencies/complaints fulfilling the definition of an ADE/SADE will also follow the processes outlined in Section 4.22 of the protocol.

### 4.24.1. Time Period for Detecting Medical Device Deficiencies/Complaints

Medical device deficiencies/complaints with or without resulting ADEs will be detected, documented, and reported during all periods of the study in which the medical device is used.
If the Investigator learns of any medical device deficiency/complaint, at any time after a participant has withdrawn from the study, and such deficiency/complaint is considered reasonably related to an investigational medical device, the Investigator will promptly notify Alexion.

### 4.24.2. Follow-up of Medical Device Deficiencies

Follow-up applies to all participants, including those who discontinue study intervention. The Investigator is responsible for ensuring that follow-up includes any supplemental investigations as indicated to elucidate the nature and/or causality of the deficiency/complaint. For each medical device deficiency/complaint, the study site must ship the OBI to Alexion, or designee, for investigation. Instructions for return of these devices will be provided in the Pharmacy Manual.

### 4.24.3. Prompt Reporting of Medical Device Deficiencies/Complaint to Alexion

Medical device deficiencies/complaint will be reported to Alexion within 1 business day after the Investigator determines that the event meets the protocol definition of a medical device deficiency/complaint.
The medical device deficiency/complaint will be reported to Alexion either by calling the Call Center using the local phone number in the IFU document or via the Complaint Report Form per the instructions in the Pharmacy Manual.
Alexion will be the contact for the receipt of Complaint Report Forms.

### 4.24.4. Regulatory Reporting Requirements for Medical Device Deficiencies

The Investigator will promptly report, within 1 business day of awareness, all medical device deficiencies/complaints occurring with any medical device provided for use in the study in order for Alexion to fulfill the legal responsibility to notify appropriate regulatory authorities and other entities about certain safety information relating to medical devices being used in clinical studies.
The Investigator, or qualified designee, will comply with the applicable local regulatory requirements relating to the reporting of medical device deficiencies/complaints to the IRB/IEC.

### 4.25. Review of Prior and Concomitant Medications and Procedures

It is important for the Investigator or designee to review each medication the participant is taking before starting the study and at each study visit (Section 1.1) and record relevant changes in the CRF.

### 4.25.1. Prior Medications and Procedures

Prior medications and/or vaccines (including vitamins, herbal preparations, and those discussed in the eligibility criteria (Section 4)) and procedures (such as mechanical ventilation, renal replacement therapy, surgery/biopsy, or physical therapy) that the participant receives or undergoes ≤28 days prior to Screening or during the Screening Period, as well as any meningococcal vaccine administered within the last 3 years, will be recorded in the participant's CRF. Transfusions of packed RBCs received during the year prior to first study intervention administration will be recorded in the participant's CRF. Prior use of eculizumab and ravulizumab (start/stop dates, dosage) will also be recorded.

### 4.25.2. Concomitant Medications and Procedures

Concomitant medications (including any medication, vitamin, herbal preparation, or supplement) and procedures (defined in Section 4.12) are those received on or after the first dose of study intervention (Day 1), including those started before Day 1 and continued after Day 1. At each study visit, participants will be questioned about any new and/or changes to medications, nonpharmacologic therapies or procedures since the last visit. Concomitant medications, nonpharmacologic therapies, and procedures will be recorded in the source documents and the participant's CRF including:
Reason for use
Dates of administration including start and end dates
Dosage information including dose and frequency

Any concomitant medication deemed necessary for the participant's care during the study, or for the treatment of any AE, along with any other medications, other than those listed as disallowed medications in Section 4.12.2, may be given at the discretion of the Investigator. However, it is the responsibility of the Investigator to ensure that details regarding all medications are recorded in full, in the participant's source documents and CRF.

Vaccination and antibiotics administered for prophylaxis of meningococcal infection (if applicable) during the study will also be recorded.

The Medical Monitor should be contacted if there are any questions regarding concomitant medications or procedures.

### 4.26. Overdose

For this study, any IV or SC dose of ravulizumab greater than that specified/required in the protocol (as outlined in Table 7 and

Table) will be considered an overdose medication error. Overdoses are medication errors that are not considered AEs unless there is an untoward medical occurrence resulting from the overdose.

There is a wide dose range of safety tolerance for ravulizumab previously clinically investigated in adults (up to 5400 mg IV, equivalent to the amount of drug in 22 full ravulizumab OBDS cartridges). Alexion does not recommend specific treatment for an overdose unless there are associated laboratory abnormalities or clinical symptoms.

In the event of an overdose or suspected overdose, the Investigator should:
Closely monitor the participant for any AE/SAE
Contact the Medical Monitor if the overdose results in an AE/SAE
Obtain a plasma sample for PK analysis if requested by the Medical Monitor (determined on a case-by-case basis)
Details of the overdose will be collected on the CRF

Decisions regarding dose interruptions or modifications will be made by the Investigator in consultation with the Medical Monitor based on the clinical evaluation of the participant.

### 4.27. Pharmacokinetics and Pharmacodynamics

Blood samples for determination of serum drug concentrations and PD assessments (free C5) will be collected before and after administration of study intervention at the time points specified in the Schedule of Activities (Section 1.1).
Instructions for the collection and handling of biological samples, including blood volume requirements, are provided in the Laboratory Manual. The actual date and time (24-hour clock time) of each sample will be recorded.
All predose PK and PD blood samples will be collected within 30 minutes prior to the start of study intervention administration. In order to minimize needle sticks to the participant, the predose sample may be drawn through the venous access created for the dose infusion, prior to administration of the ravulizumab IV dose on Day 1 (if applicable).
All postdose PK and PD blood samples will be collected within 60 minutes after completion of the study intervention administration. The postdose blood samples will be drawn from the participant's opposite, noninfused arm on Day 1 (for participants administered a ravulizumab IV dose).
For indicated visits not falling on dosing days, samples may be collected at any time that visit day.

### 4.28. Genetics

### 4.29. Biomarkers

### 4.30. Immunogenicity Assessments

Serum samples for immunogenicity analysis will be collected at time points according to the Schedule of Activities (Section 1.1). All efforts will be made to obtain the immunogenicity samples at the exact nominal time relative to dosing. Out-of-window protocol deviation capture for immunogenicity samples follows that specified for PK sample collection (Section 4.27).

The detection and characterization of ADA to ravulizumab will be performed using a validated assay method by or under the supervision of Alexion. Antibodies to ravulizumab will be evaluated in serum samples collected from all participants according to the Schedule of Activities (Section 1.1). ADA positive samples will be further characterized for antibody titer and presence of neutralizing antibodies. Samples may be stored for a maximum of 25 years (or according to local regulations) following the last participant's last visit for the study at a facility selected by Alexion. Additional analyses may be performed on collected ADA samples for further analysis or characterization.

### 4.31. Device Performance Assessment

Device performance will be assessed using the reported outcome of attempted full dose administration via ravulizumab OBDS (including device failure/malfunction) per the requirements in the IFU.

Investigators and participants/caregivers must report all device deficiencies/complaints observed during the course of the study. Device deficiencies/complaints will be documented in the CRF as appropriate.

In the event of a device deficiency/complaint, whether or not associated with a missed dose, the ravulizumab OBDS must be sent to a core laboratory for analysis.

### 5. Statistical Considerations

### 5.1. Statistical Hypotheses

No statistical hypothesis testing is planned.

### 5.2. Sample Size Determination

The sample size for this study is not based on statistical considerations. The number of participants has been chosen based on feasibility and is considered adequate to meet the primary objective. This study plans to enroll approximately 22 participants in order to obtain at least 20 evaluable pediatric participants with either PNH or aHUS who have received at least 1 dose of ravulizumab SC and have evaluable PK data at Day 1 postdose, Day 15 predose, Day 15 postdose, or Day 71 predose.

### 5.3. Analysis Sets

The participant analysis sets are defined in Table 8.

TABLE 8
Participant Analysis Sets

| Participant Analysis Set | Description |
|---|---|
| Enrolled Set | All consented participants excluding screen failures. |
| PK Analysis Set | All enrolled participants who receive at least 1 dose of ravulizumab SC |
| | and who have evaluable^{a} concentration values at Day 1 postdose, Day |
| | 15 predose, Day 15 postdose, or Day 71 predose |
| PD Analysis Set | All enrolled participants who receive at least 1 dose of ravulizumab SC |
| | and who have evaluable^{a} PD values at Day 1 postdose, Day 15 |
| | predose, Day 15 postdose, or Day 71 predose |
| Modified Full Analysis Set | PNH Cohort: All enrolled participants who receive at least 1 dose of |
| | ravulizumab SC and who have at least 1 postbaseline secondary |
| | efficacy or QoL assessment |
| | aHUS Cohort: For this cohort, confirmation of eligibility in participants |
| | may occur after receiving study intervention. Therefore, the Modified |
| | FAS for the aHUS Cohort consists of all participants who receive at |
| | least 1 dose of study intervention, who have at least 1 postbaseline |
| | efficacy or QoL assessment and meet all of the following criteria: |
| | Participants who satisfy Exclusion Criterion 27 |
| | Participants who satisfy Exclusion Criterion 28 |
| Safety Set | All enrolled participants who receive at least 1 dose of ravulizumab SC |
| Immunogenicity Analysis Set | All participants who receive at least 1 dose of ravulizumab SC and |
| (IAS) | have at least 1 reportableb post-dose result in the ADA assay. |

Note:
Participants who discontinue study intervention after receiving a ravulizumab IV loading dose and do not receive a ravulizumab SC dose will not be included in any of the analysis sets. If applicable, data for these participants will be included in the data listings.
^{a}Evaluable PK and PD data are defined as nonmissing results generated from samples that comply with sample integrity requirements during sample collection, storage, shipment, and bioanalysis.
^{b} Reportable result in the ADA assay means that a sample has been given a clear positive or negative status in the ADA assay.
Abbreviations:
ADA = antidrug antibody;
aHUS = atypical hemolytic uremic syndrome;
FAS = Full Analysis Set;
ICF = informed consent form;
IV = intravenous;
PD = pharmacodynamic;
PK = pharmacokinetic;
PNH = paroxysmal nocturnal hemoglobinuria;
QoL = quality of life;
SC = subcutaneous

### 5.4. Statistical Analyses

Summary statistics will be computed and displayed by visit, where applicable. Descriptive statistics for continuous variables will minimally include the number of participants, mean, standard deviation (SD), minimum, median, and maximum. For categorical variables, frequencies, and percentages will be presented. 95% confidence intervals (Cls) may be included for select endpoints as appropriate. Graphical displays will be provided as appropriate.

Descriptive statistics for PK data will include the number of participants, mean, SD, coefficient of variation (CV), median, minimum, maximum, geometric mean, and geometric mean % CV.

All analyses are descriptive in nature and no adjustment for multiplicity will be performed.

Analyses will be performed using the SAS® software Version 9.4 or higher.

In general, the PK and PD analyses will be conducted for the complement inhibitor treatment-naïve and eculizumab-experienced (i.e., participants receiving a loading dose of ravulizumab IV followed by ravulizumab SC maintenance dosing) subgroup versus ravulizumab-experienced subgroup (i.e., participants receiving ravulizumab SC maintenance dosing only) for each disease cohort separately. Additionally, subgroup analyses will be performed by weight cohort (<20 kg versus ≥20 kg) for the complement inhibitortreatment-naïve/eculizumab-experienced subgroup and the ravulizumab-experienced subgroup for each disease cohort.

Safety, efficacy, and quality of life endpoints will be analyzed for the complement inhibitor treatment-naïve subgroup and complement inhibitor-experienced (eculizumab-experienced and ravulizumab-experienced) subgroup for each disease cohort separately as applicable.

Analyses for device performance will be conducted for all participants who receive ravulizumab SC via the ravulizumab OBDS for each disease cohort separately.

Table 9 displays the previous treatment experience subgroups to be used for each endpoint analysis for each disease cohort.

TABLE 9
Previous Treatment Experience Subgroup Definitions to
be Used for Each Endpoint for Each Disease Cohort

| | PK, PD, | | Efficacy | Device |
|---|---|---|---|---|
| Subgroup | and ADA | Safety | and QoL | Performance |
| Complement inhibitor treatment-naïve | X | | | |
| participants/eculizumab-experienced | | | | |
| participants (i.e., participants who | | | | |
| received ravulizumab IV loading dose | | | | |
| followed by ravulizumab SC maintenance | | | | |
| dosing) | | | | |
| Ravulizumab-experienced participants | X | | | |
| (i.e., participants who received | | | | |
| ravulizumab SC maintenance dosing and | | | | |
| no ravulizumab IV loading dose) | | | | |
| Complement inhibitor treatment-naïve | | X | X | |
| participants | | | | |
| Complement inhibitor | | X | X | |
| treatment-experienced participants | | | | |
| (eculizumab-experienced and | | | | |
| ravulizumab-experienced) | | | | |
| All participants treated with ravulizumab | | | | X |
| SC via ravulizumab OBDS as applicable | | | | |

Abbreviations:
ADA = antidrug antibody;
IV = intravenous;
OBDS = on-body delivery system;
PD = pharmacodynamic;
PK = pharmacokinetic;
QoL = quality of life;
SC = subcutaneous

### 5.4.1. Pharmacokinetic and Pharmacodynamic Analyses

The primary PK analysis will be based on the PK Analysis Set and will be conducted after all participants have completed the Primary Evaluation Period, or discontinued study intervention during the Primary Evaluation Period.

Any participant PK data collected after the following 2 intercurrent events will be excluded from the primary PK analysis:
Discontinuation of study intervention prior to the end of the Primary Evaluation Period (Day 71)
Initiation of specific disallowed medication or therapy prior to the end of the Primary Evaluation Period (Day 71) as described in Section 4.12.2.

Individual serum concentration data for all participants in the PK Analysis Set will be used to generate descriptive statistics to summarize the serum concentration data at each sampling time point. Graphs of mean serum concentration-time profiles and graphs of serum concentration-time profiles for individual participants may be provided, as appropriate. Actual dose administration and sampling times will be used for all calculations.

The PD effects of ravulizumab will be evaluated by assessing the absolute values and changes and percentage changes from baseline in serum free C5 concentrations over time, as appropriate using the PD Analysis Set. Descriptive statistics will be calculated for the PD data at each sampling time, as appropriate. Any participant PD data collected after an intercurrent event as described similar to above will be excluded from the primary PD analysis.

Exploratory assessments of PK/PD relationships may be conducted.

### 5.4.2. Efficacy Analyses

The secondary efficacy analyses will be descriptive in nature and will be based on the Modified Full Analysis Set (mFAS). Continuous efficacy endpoints will be evaluated using descriptive statistics and categorical efficacy endpoints will be summarized using frequencies and percentage of participants. It has been observed in other studies that up to 1% of central laboratory chemistry samples undergo in vitro erythrocyte lysis referred to as table-top hemolysis (TTH) caused by sample mishandling. Due to the artefactual increase in LDH in samples affected by TTH, the potassium alanine transaminase, aspartate transaminase, magnesium, phosphorus, and LDH values in samples affected by TTH will not be used in the analysis of any efficacy or safety endpoints with the exception that the LDH values will be used for the qualification of breakthrough hemolysis. TTH samples from the central lab will be defined as having serum potassium 6 mmol/L and LDH ≥2×ULN and will be excluded from analyses as described above.

### 5.4.3. Quality of Life

The following quality of life assessments will be summarized at Baseline and each post-baseline timepoint using descriptive statistics for continuous variables for the observed and change from baseline for the following assessments:
Pediatric FACIT-Fatigue scale (participants 8 years of age at time of consent only)
PedsQL 4.0 Generic Core Scale scores
   For participants ≥5 years of age at time of assessment, both participants and caregivers will be evaluated.
   For participants 2 to 4 years of age at time of assessment, only caregivers will complete the questionnaire.

### 5.4.4. Safety Analyses

All safety analyses will be performed on the Safety Set.

Participants who discontinue study intervention after receiving a ravulizumab IV loading dose will not be included in the Safety Set and the safety data will be reported separately.

### 5.4.4.1. Adverse Events and Adverse Device Effects

The following definitions will be used:
Pretreatment AEs: Any AE that starts after providing informed consent, but before the first dose of study intervention
Treatment-emergent adverse event (TEAE): Any TEAE that starts during or after the start of the first dose of study intervention (ravulizumab IV loading dose or SC maintenance dose).
Treatment-emergent SAE (TESAE): A TEAE that meets the criteria for serious
Adverse device effect: An AE considered to be related with the investigational medical device and/or the use of the device. ADEs are a subset of AEs. All ADEs are by definition occurring during or after the start of the first dose administration via ravulizumab OBDS.

The incidence of treatment-emergent AEs, SAEs, and AEs leading to study intervention discontinuation will be summarized.

The incidence of ADEs, serious ADEs, and ADEs leading to study intervention discontinuation will also be summarized separately for participants in the Safety Set that receive ravulizumab SC via ravulizumab OBDS (i.e., participants who weigh ≥20 kg).

All AEs will be coded using the Medical Dictionary for Regulatory Activities (MedDRA), version 25 or higher, and will be summarized by System Organ Class and Preferred Term overall, by severity, and by relationship to study intervention.

Detailed by-participant listings of TEAEs (including TEAEs related to study intervention), ADEs (including serious ADEs), TESAEs, and TEAEs leading to study intervention discontinuation will be provided.

### 5.4.4.2. Analysis of Clinical Laboratory Parameters, Vital Sign Measurements, and Electrocardiogram Parameters

Laboratory measurements as well as their changes from baseline at each visit and shift from baseline, if applicable, will be summarized descriptively.

Changes from baseline in vital signs, weight, and height for all participants will be summarized descriptively. Changes from baseline in head circumference for participants 3 years of age will be summarized descriptively.

ECGs, including ECG interpretation heart rate, PR, QRS, QT, and QTc intervals will also be summarized.

### 5.4.5. Immunogenicity Analysis

All immunogenicity analyses will be performed on the Immunogenicity Analysis Set. Immunogenicity variables include ADA status, ADA response category, and ADA or neutralizing antibody (Nab) incidence and titer over the duration of the study.
ADA status categories
   ADA negative
   ADA positive
Participants who are ADA positive will be further categorized into ADA response categories as follows:
   Pre-existing immunoreactivity
   Treatment-emergent ADA responses
   Treatment-boosted ADA responses
Participants with treatment-emergent or treatment-boosted ADA responses will be further categorized as:
   Persistent responses
   Indeterminate responses
   Transient responses
ADA positive samples will be further characterized for neutralizing activity in the Nab assay. Nab status categories are as follows:
   Nab positive
   Nab negative

### 5.4.6. Device Performance Analysis

Device performance outcomes will be summarized using the Safety Set but only for participants who receive ravulizumab SC administration using the ravulizumab OBDS.

The number and percentage of full dose administrations, devices which delivered their full volume, and devices with a reported device deficiency/complaint, along with the root cause categories, will be summarized.

### 5.5. Interim Analyses

An interim analysis will be conducted to confirm or adjust the SC dosing regimen in pediatric participants. This analysis will be performed after 4 pediatric participants weighing between 10 kg to <40 kg (with a minimum of 2 patients weighing <20 kg) have completed study intervention through Day 29. The interim analysis will include all available data at the time of the data cutoff date. Enrollment of participants will proceed without interruption while the analysis is ongoing.

The primary PK analysis will be performed at the end of the 10-week Primary Evaluation Period (i.e., after all participants in each disease-specific cohort have completed the Day 71 Visit or discontinued study intervention prior to completing the 10-week Primary Evaluation Period). This analysis will allow for evaluation of the primary endpoint and will have no impact on the progression of this study.

The final analysis will be performed at the end of the study (i.e., after all participants have completed or withdrawn from the study).

### 5.6. Data Monitoring Committee

### Supporting Documentation and Operational Considerations

### 5.7. Regulatory, Ethical, and Study Oversight Considerations

### 5.7.1. Regulatory and Ethical Considerations

### 5.7.2. Financial Disclosure

### 5.7.3. Informed Consent and Assent Process

### 5.7.4. Recruitment Strategy

### 5.7.5. Data Protection

### 5.7.6. Dissemination of Clinical Study Data

### 5.7.7. Data Quality Assurance

### 5.7.8. Source Documents

### 5.7.9. Study and Site Start and Closure

### 5.7.10. Publication Policy

### 5.7.11. Good Clinical Practice Compliance

### Participant-Reported Outcome Instruments: Samples are Provided in FIGS. 5, 6A, 6B, 7A-7C, 8A, 8B, 9A, 9B, 10A, 10B, 11A, 11B, 12A, and 12B.

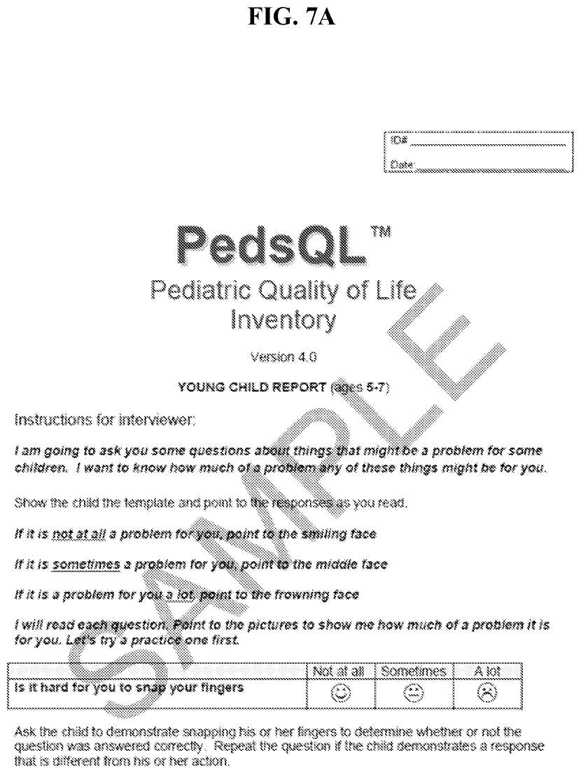

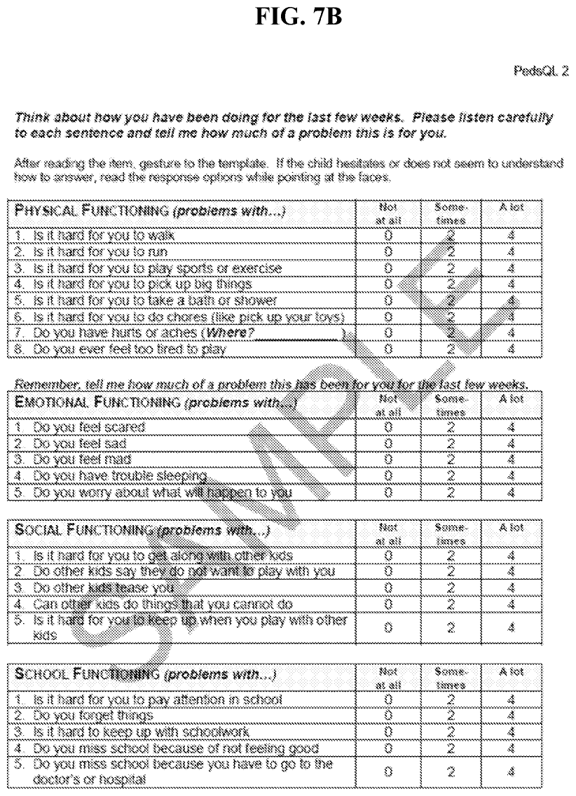

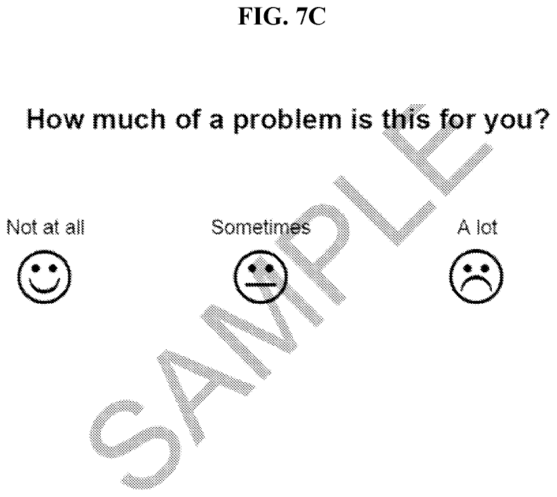

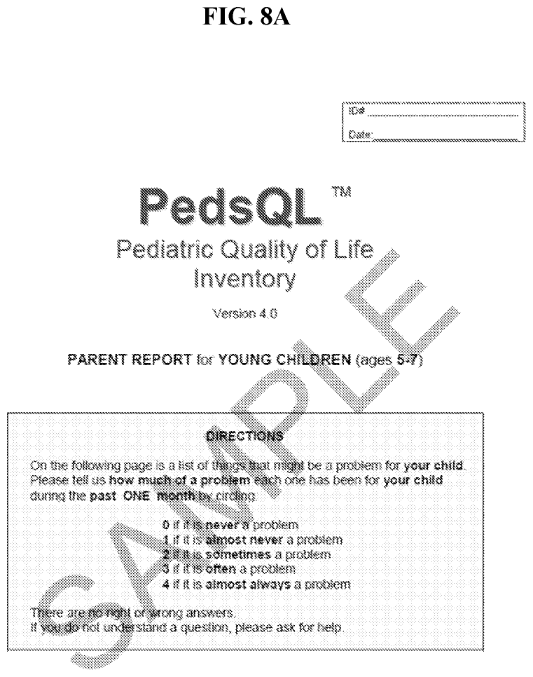

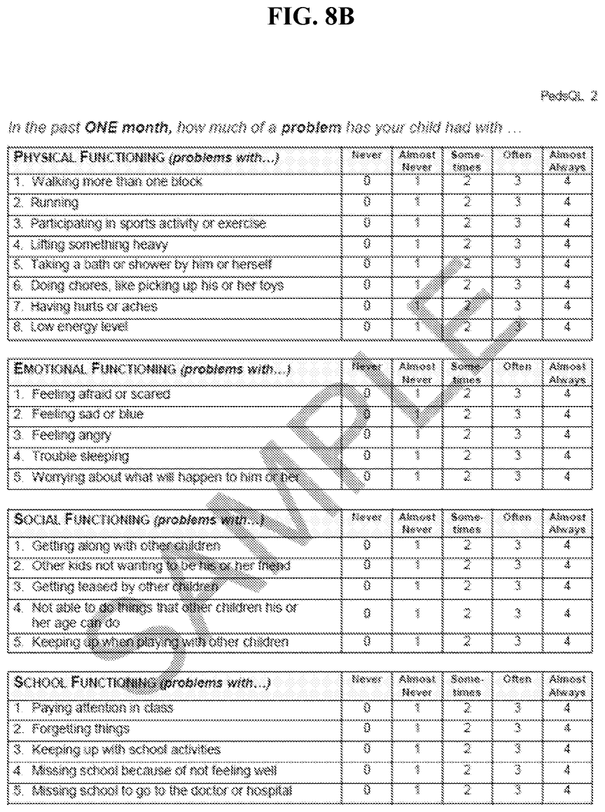

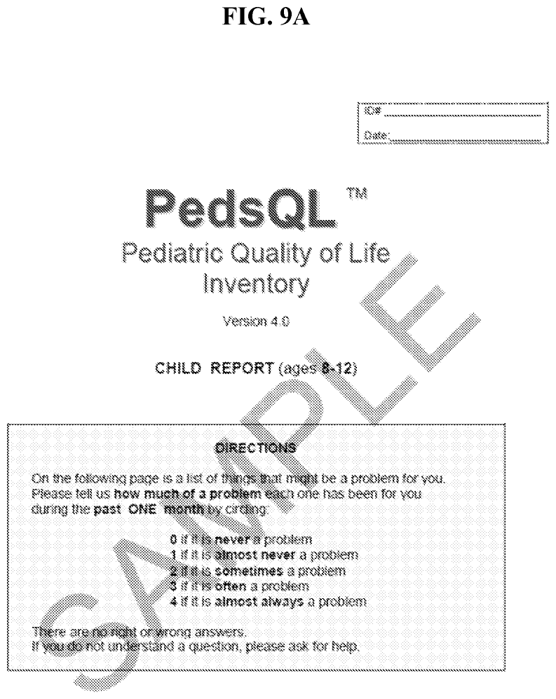

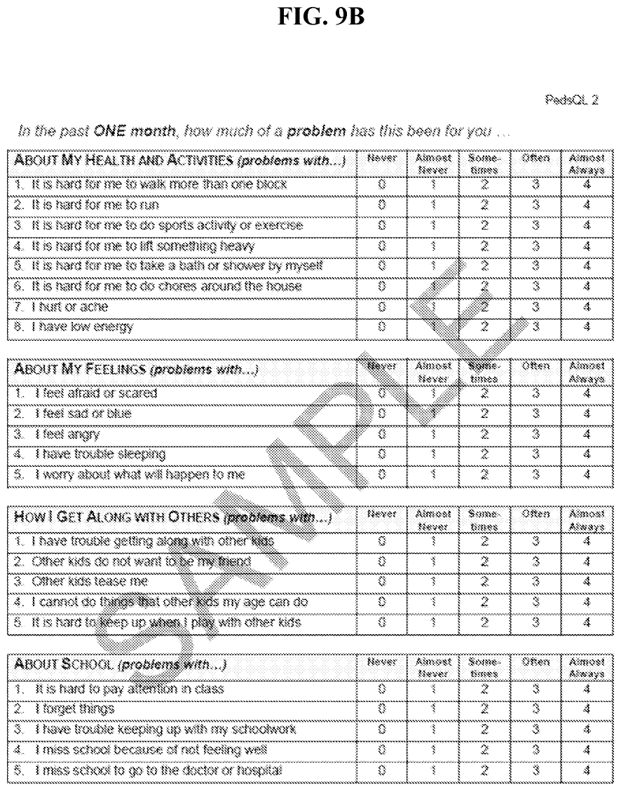

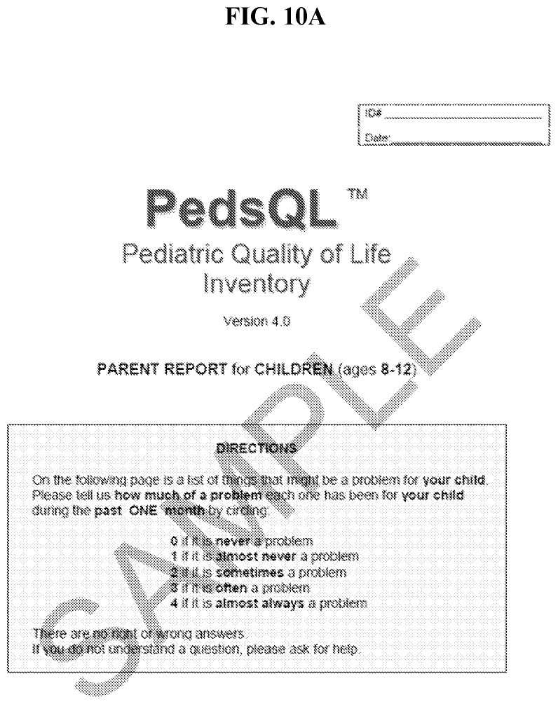

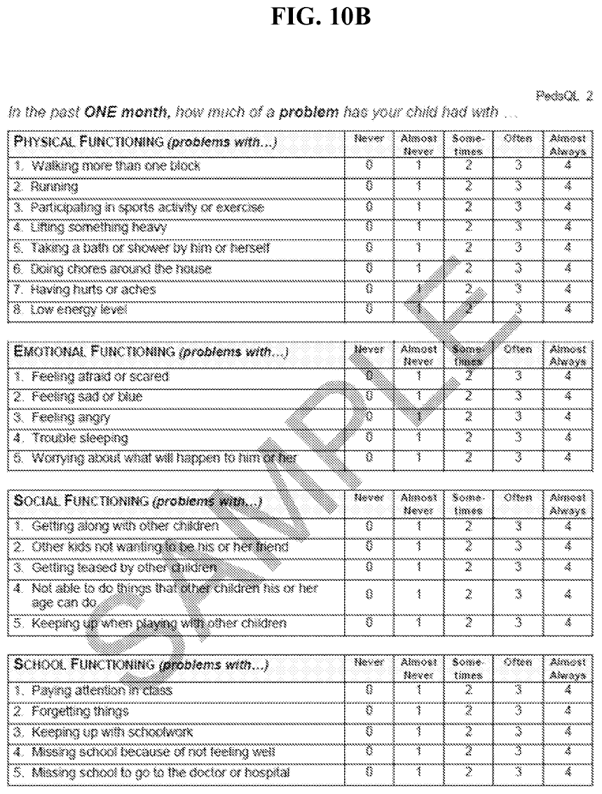

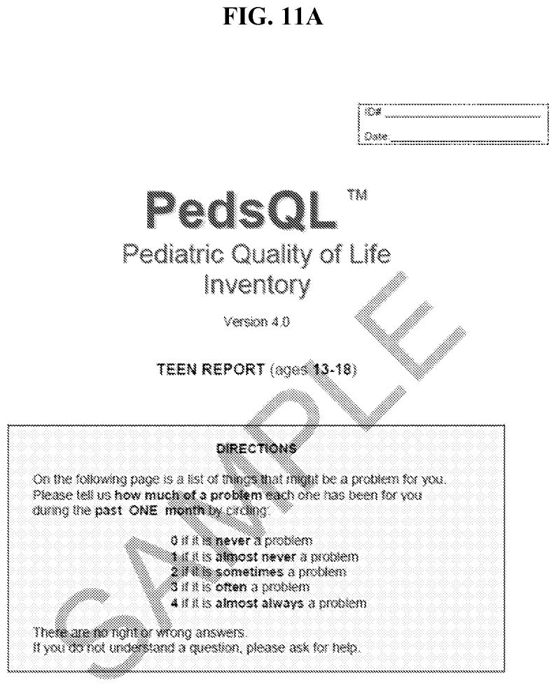

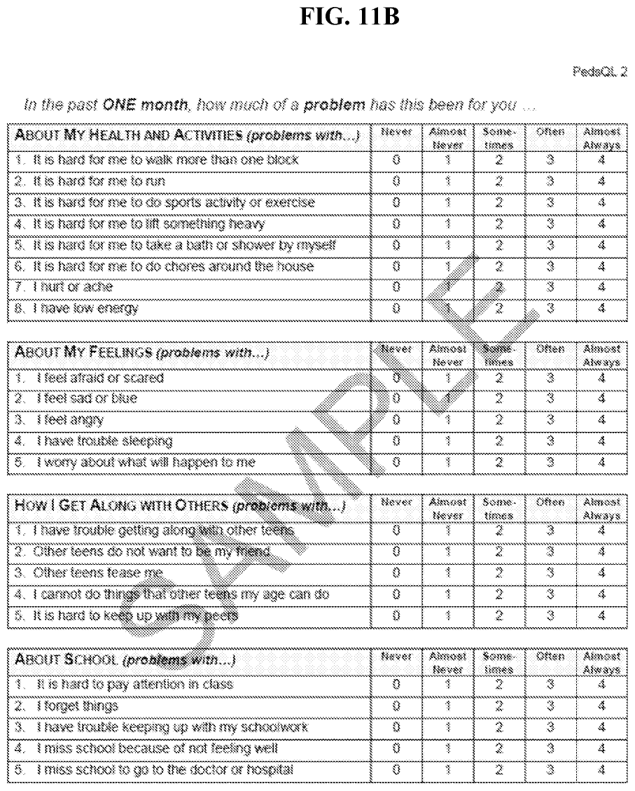

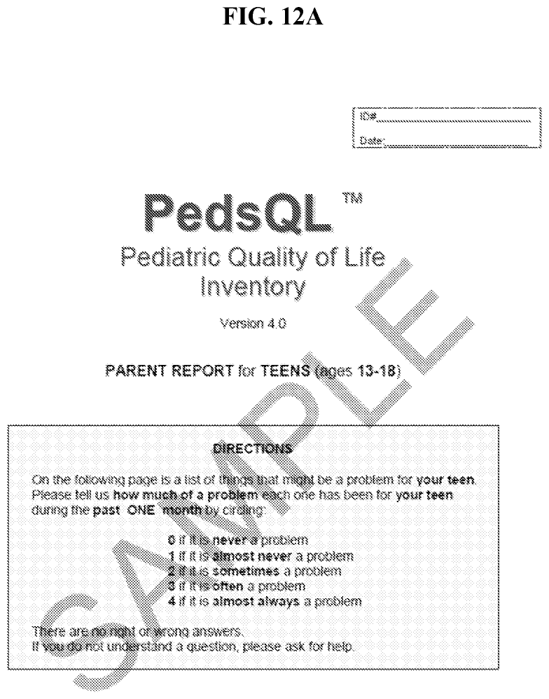

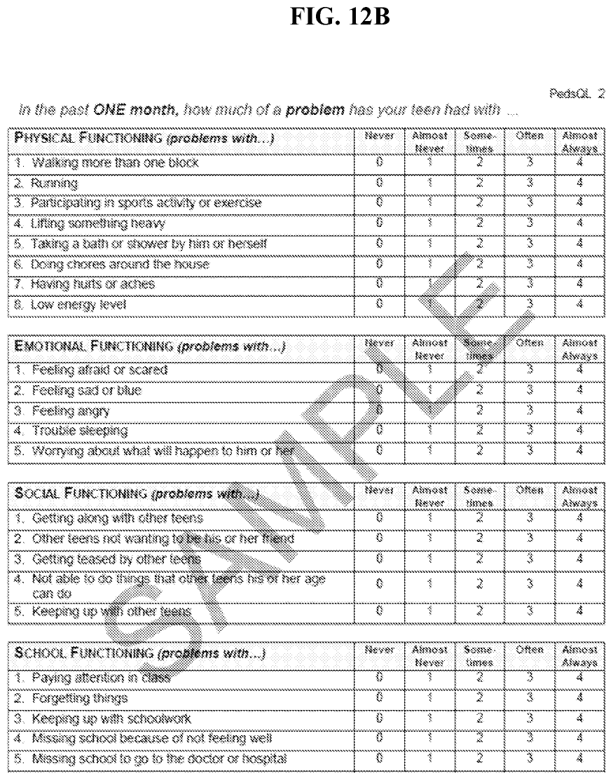

5.7.12. Pediatric Functional Assessment of Chronic Illness Therapy—Fatigue (see FIG. 5)
5.7.13. Pediatric Quality of Life Inventory (PedsQL) 4.0 Generic Core Scale
5.7.13.1. Parent Report for Toddlers (Ages 2-4) (See FIGS. 6A and 6B)
5.7.13.2. Young Child Report (Ages 5-7) (See FIGS. 7A-7C)
5.7.13.3. Parent Report for Young Children (Ages 5-7) (FIGS. 8A and 8B)
5.7.13.4. Child Report (Ages 8-12) (FIGS. 9A and 9B)
5.7.13.5. Parent Report for Children (Ages 8-12) (FIGS. 10A and 10B)
5.7.13.6. Teen Report (Ages 13-18) (FIGS. 11A and 11B)
5.7.13.7. Parent Report for Teens (Ages 13-18) (FIGS. 12A and 12B)

### 5.8. Clinical Laboratory Tests

The tests detailed in Table 11 will be performed by the central laboratory or designated ancillary laboratories.
Local laboratory results are only required in the event that the central laboratory results are not available in time for either study intervention administration and/or response evaluation. If a local sample is obtained, it is important that the sample for central analysis is obtained at the same time. Additionally, if the local laboratory results are used to make either a study intervention decision or response evaluation, the results must be entered into the CRF.
Protocol-specific requirements for inclusion or exclusion of participants are detailed herein.
Additional tests may be performed at any time during the study as determined necessary by the Investigator or required by local regulations.
Investigators must document their review of each laboratory safety report.

TABLE 11
Protocol-Required Laboratory Assessments

| Laboratory Assessments | Parameters |
|---|---|
| Hematology | Haptoglobin |
| | Hematocrit |
| | Hemoglobin |
| | Platelet count |
| | RBC count |
| | RBC indices |
| | Distribution width |
| | Mean corpuscular volume |
| | Mean corpuscular hemoglobin |
| | Percentage of reticulocytes |
| | WBC count with differential |
| | Neutrophils |
| | Lymphocytes |
| | Monocytes |
| | Eosinophils |
| | Basophils |
| Coagulation panel | D-dimer |
| | INR |
| | Partial thromboplastin time |
| | Prothrombin time |
| Clinical chemistry | Alanine aminotransferase |
| | Albumin |
| | Alkaline phosphatase |
| | Aspartate aminotransferase |
| | Bicarbonate |
| | Blood urea nitrogen |
| | Calcium |
| | Chloride |
| | C-reactive protein |
| | Gamma-glutamyltransferase |
| | Glucose (nonfasting) |
| | Lactate dehydrogenase |
| | Magnesium |
| | Phosphorus |
| | Potassium |
| | Serum creatinine (note: eGFR will be calculated using Modification |
| | of Diet in Renal Disease Schwartz formula) |
| | Sodium |
| | Total and direct bilirubin |
| | Total protein |
| | Uric acid |
| Routine urinalysis | Appearance |
| | Bilirubin |
| | Blood |
| | Color |
| | Creatinine |
| | Glucose |
| | Ketones |
| | Nitrite |
| | pH |
| | Protein |
| | Protein creatinine ratio |
| | Specific gravity |
| | Urobilinogen |
| Other tests | PNH clone size (PNH cohort only) |
| PK/PD/Immunogenicity | Serum PK |
| | Free C5 |
| | ADA |
| Other study-specific tests | Serum or urine human chorionic gonadotropin pregnancy test (as |
| | needed for WOCBP)^{a} |
| | ADAMTS13 activity^{b} (aHUS cohort only) |
| | Coombs, direct^{b} (aHUS cohort only) |
| | Shiga toxin-related HUS screen^{b} (e.g., Shiga toxin enzyme |
| | immunoassay/PCR in stool/stool culture (aHUS cohort only) |

^{a}Serum pregnancy test required at Screening, Day 71, Week 52, and ED Visit (if applicable); local urine pregnancy test at all other times as specified in Schedule of Activities.
^{b}For eculizumab-experienced or ravulizumab-experienced participants with aHUS, historical test results via chart review should be utilized.
Abbreviations:
ADA = antidrug antibody;
ADAMTS13 = a disintegrin and metalloproteinase with a thrombospondin type 1 motif;
aHUS = atypical hemolytic uremic syndrome;
C5 = complement component 5;
ED = early discontinuation;
eGFR = estimated glomerular filtration rate;
INR = international normalized ratio;
PCR = polymerase chain reaction;
PD = pharmacodynamic;
PK = pharmacokinetic;
PNH = paroxysmal nocturnal hemoglobinuria;
RBC = red blood cell;
WBC = white blood cell;
WOCBP = women of childbearing potential

### 5.9. Management of Potential Infusion-Associated Adverse Events During Study Intervention Administration

Intravenous and SC infusion-associated reactions are a potential risk with the use of monoclonal antibodies; these reactions can be nonimmune or immune mediated (e.g., hypersensitivity reactions). Signs and symptoms may include headache, fever, facial flushing, pruritus, myalgia, nausea, chest tightness, dyspnea, vomiting, erythema, abdominal discomfort, diaphoresis, shivers, hypertension, lightheadedness, hypotension, palpitations, and somnolence. Signs and symptoms of hypersensitivity or allergic reactions may include hives, swollen face, eyelids, lips, or tongue, or trouble with breathing.

All administration-, IV-, and infusion-associated reactions will be reported to the Investigator and qualified designee. The Investigator and qualified designee are responsible for detecting, documenting, and recording events that meet the definition of AE or SAE and remain responsible for following up events that are serious, considered related to the study intervention, or study procedures; or that caused the participant to discontinue the study intervention.

Definitions and procedures for recording, evaluating, follow-up, and reporting AEs and SAEs are outlined herein.

Participants who experience a reaction during the administration of study intervention should be treated according to institutional guidelines.

Participants who experience a severe reaction during administration of study intervention resulting in discontinuation of study intervention will undergo all scheduled safety, PK, and PD evaluations required by the protocol. Alexion, or designee, must be notified within 24 hours of any infusion reaction requiring interruption or discontinuation of study intervention. All AEs that may indicate an infusion related response will be graded according to the CTCAE v5.0 or higher.

If anaphylaxis occurs according to the criteria listed in Table 12, then administration of SC epinephrine ( 1/1000, 0.15 mL to 0.5 mL, or equivalent) should be considered. In the case of bronchospasm, treatment with an inhaled beta agonist also should be considered. Participants administered an antihistamine for the treatment or prevention of an infusion reaction should be given appropriate warnings about drowsiness and impairment of driving ability before being discharged from the center.

TABLE 12
Clinical Criteria for Diagnosing Anaphylaxis
Anaphylaxis is highly likely when any 1 of the following 3 criteria is fulfilled:

Acute onset of an illness (minutes to several hours) with involvement of the skin, mucosal tissue,
or both (e.g., generalized hives, pruritus or flushing, swollen lips-tongue-uvula), and at least 1 of
the following:
Respiratory compromise (e.g., dyspnea, wheeze-bronchospasm, stridor, reduced PEF,
hypoxemia)
Reduced BP or associated symptoms of end-organ dysfunction (e.g., hypotonia (collapse),
syncope, incontinence)
Two or more of the following that occur rapidly after exposure to a likely allergen (minutes to
several hours):
Involvement of the skin-mucosal tissue (e.g., generalized hives, itch-flush, swollen
lips/tongue/uvula)
Respiratory compromise (e.g., dyspnea, wheeze/bronchospasm, stridor, reduced PEF,
hypoxemia)
Reduced BP or associated symptoms (e.g., hypotonia [collapse], syncope, incontinence)
Persistent gastrointestinal symptoms (e.g., crampy abdominal pain, vomiting)
Reduced BP after exposure to known allergen (minutes to several hours):
Infants and children: low systolic BP (age specific) or greater than 30% decrease in systolic
BP
Abbreviations:
BP = blood pressure;
PEF = peak expiratory flow

### 5.10. Blood Sampling Volumes

The following procedures for blood collection should be adhered to:
Number of attempts: The number of attempts for sampling blood is limited to 3 times per day. This means that, after 3 punctures for collection of blood have been performed and no or insufficient blood could be collected, no other puncture will be done on the same day.
Volume of blood samples: Per study participant, the study-related blood loss (including any losses in the collection procedure) should not exceed 3% of the total blood volume during a period of 4 weeks and should not exceed 1% at any single time. The total volume of blood is estimated at 80 to 90 mL/kg body weight. Three percent (3%) is 2.4 mL blood per kg of body weight. If an Investigator decides to deviate from these limits, the deviation must be fully documented, and the Investigator should provide justification for the deviation. If the required blood volume cannot be obtained, due to the above-mentioned safety limits, priority will be given to PK/PD relevant investigations.
Eutectic mixture of local anesthetics (EMLA) cream/plaster: To minimize the possible pain and discomfort due to collection of blood, the Investigator may apply an EMLA cream/plaster at the puncture site.
   Reference: European Commission. European Commission Ethical Considerations for Clinical Trials on Medicinal Products Conducted with the Paediatric Population: Recommendations of the ad hoc group for the development of implementing guidelines for Directive 2001/20/EC relating to the good clinical practice in the conduct of clinical trials on medicinal products for human use. 2008.

### ADDITIONAL EMBODIMENTS

1. An on-body delivery system (OBDS) configured for administration of an effective amount of a therapeutic anti-C5 antibody, or antigen-binding fragment thereof, comprising complementarity determining region (CDR) 1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat atypical hemolytic uremic syndrome (aHUS) or paroxysmal nocturnal hemoglobinuria (PNH) in a pediatric human patient in need thereof, wherein the patient is complement inhibitor treatment-naïve or eculizumab-experienced, wherein the therapeutic anti-C5 antibody, or antigen-binding fragment thereof, is administered:
   subcutaneously once per week at a dose of:
      (i) 245 mg to a patient weighing ≥20 to <40 kg; or
      (ii) 490 mg to a patient weighing ≥40 kg.
2. An OBDS configured for treating aHUS or PNH in a pediatric human patient in need thereof, wherein the OBDS comprises an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is complement inhibitor treatment-naïve or eculizumab-experienced, and wherein the OBDS comprises means for subcutaneous administration of:
   (i) 245 mg to a patient weighing ≥20 to <40 kg; or
   (ii) 490 mg to a patient weighing ≥40 kg.
3. An OBDS configured for administration of an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat aHUS or PNH in a pediatric human patient in need thereof, wherein the patient is ravulizumab-experienced, wherein the therapeutic anti-C5 antibody, or antigen-binding fragment thereof, is administered:
   subcutaneously once per week at a dose of:
      (i) 245 mg to a patient weighing ≥20 to <40 kg; or
      (ii) 490 mg to a patient weighing ≥40 kg.
4. An OBDS configured for treating aHUS or PNH in a pediatric human patient in need thereof, wherein the OBDS comprises an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ravulizumab-experienced, and wherein the OBDS comprises means for subcutaneous administration of:
   (i) 245 mg to a patient weighing ≥20 to <40 kg; or
   (ii) 490 mg to a patient weighing ≥40 kg.
5. The OBDS of any one of embodiments 1-4, wherein the pediatric human patient is less than 21 years of age.
6. The OBDS of embodiment 5, wherein the pediatric human patient is between 2 and 18 years of age.
7. The OBDS of any one of embodiments 1-6, wherein the OBDS comprises a cartridge comprising the therapeutic anti-C5 antibody, or antigen-binding fragment thereof, and an on-body injector (OBI).
8. The OBDS of any one of embodiments 1-7, wherein the OBDS is configured for administration of the therapeutic anti-C5 antibody, or antigen-binding fragment thereof, subcutaneously to a patient weighing ≥20 to <40 kg at a dose of 245 mg using one OBDS.
9. The OBDS of embodiment 8, wherein the 245 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% weight by volume (w/v) sucrose, 0.05% w/v polysorbate 80 (PS80), and water for injection, and wherein the OBDS comprises one OBI and one pre-filled cartridge containing 245 mg of the therapeutic anti-C5 antibody or antigen-binding fragment thereof.
10. The OBDS of any one of embodiments 1-7, wherein the OBDS is configured for administration of the therapeutic anti-C5 antibody, or antigen-binding fragment thereof, subcutaneously to a patient weighing ≥40 kg at a dose of 490 mg in combination with a second OBDS.
11. The OBDS of embodiment 10, wherein the 490 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein each OBDS comprises one OBI and one pre-filled cartridge containing 245 mg of the therapeutic anti-C5 antibody or antigen-binding fragment thereof.
12. The OBDS of any one of embodiments 1-11, wherein the OBDS is a single-use electro-mechanical medical device comprising: an injector comprising a 29-gauge needle and a cartridge comprising a piston and telescopic screw assembly.
13. The OBDS of any one of embodiments 7-12, wherein the cartridge has a volume of 3.5 mL.
14. The OBDS of any one of embodiments 7-13, wherein the OBDS is configured to deliver the therapeutic anti-C5 antibody, or antigen-binding fragment thereof, via subcutaneous injection in about 10 minutes.
15. The OBDS of any one of embodiments 7-14, wherein the cartridge comprises the therapeutic anti-C5 antibody, or antigen-binding fragment thereof, and an opening sealed by a pierceable septum.
16. The OBDS of any one of embodiments 1-15, wherein the OBDS comprises an OBI comprising:
   (i) an injector housing;
   (ii) an injection needle translatable between a retracted position, wherein at least a tip of the injection needle is contained within the injector housing, and an injection position, wherein at least the tip of the injection needle protrudes from the injector housing;
   (iii) an activation button assembly movably mounted to the injector housing and operatively connected to the injection needle, the activation button assembly being translatable from an unactuated position to an actuated position to drive the injection needle from the retracted position thereof to the injection position thereof;
   (iv) a cartridge door movably mounted to the injector housing between an open position and a closed position, the cartridge door comprising:
      an open end,
      an interior channel having a cartridge mounted therein, the cartridge containing the therapeutic anti-C5 antibody, or antigen-binding fragment thereof, to be dispensed and having an opening at a front end of the cartridge sealed by a pierceable septum and a flange at a rear end thereof, and
      a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and
   (v) a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum;
   wherein the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum.
17. The OBDS of embodiment 15 or 16, wherein the cartridge has a volume of 10 mL.
18. The OBDS of any one of embodiments 1-17, wherein the OBDS is configured for self-administration of the therapeutic anti-C5 antibody, or antigen-binding fragment thereof, subcutaneously to the patient.
19. The OBDS of any one of embodiments 1-18, wherein the OBDS is configured for subcutaneous administration of the therapeutic anti-C5 antibody, or antigen-binding fragment thereof, to a site selected from the patient's arm, abdomen, and thigh.
20. A needle syringe configured for administration of an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat aHUS or PNH in a pediatric human patient in need thereof, wherein the patient is ≥10 to <20 kg of body weight and is complement inhibitor treatment-naïve, eculizumab-experienced, or ravulizumab-experienced, wherein the therapeutic anti-C5 antibody, or antigen-binding fragment thereof, is administered: subcutaneously once per week at a dose of 150 mg.
21. A needle syringe configured for treating aHUS or PNH in a pediatric human patient in need thereof, wherein the needle syringe is configured for subcutaneous administration and comprises 150 mg of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ≥10 to <20 kg of body weight and is complement inhibitor treatment-naïve, eculizumab-experienced, or ravulizumab-experienced.
22. The needle syringe of embodiment 20 or 21, wherein the 150 mg dose of the therapeutic anti-C5 antibody, or antigen-binding fragment thereof, is formulated at pH 7.4 and a concentration of 100 mg/mL in sodium phosphate, sucrose, L-arginine, PS80, and water for injection.
23. The needle syringe of any one of embodiments 20-22, wherein the needle syringe is configured for administration of the therapeutic anti-C5 antibody, or antigen-binding fragment thereof, to a site selected from the patient's arm, abdomen, and thigh.
24. The OBDS of any one of embodiments 1-19, or the needle syringe of any one of embodiments 20-23, wherein the therapeutic anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.
25. The OBDS of any one of embodiments 1-19 and 24, or the needle syringe of any one of embodiments 20-23 and 24, wherein the therapeutic anti-C5 antibody, or antigen-binding fragment thereof, comprises a heavy chain variable region comprising the amino acid sequence of SEQ ID NO: 12 and a light chain variable region comprising the amino acid sequence of SEQ ID NO: 8.
26. The OBDS of any one of embodiments 1-19, 24, and 25, or the needle syringe of any one of embodiments 20-25, wherein the therapeutic anti-C5 antibody, or antigen-binding fragment thereof, further comprises a heavy chain constant region comprising the amino acid sequence of SEQ ID NO: 13.
27. The OBDs of any one of embodiments 1-19 and 24-26, or the needle syringe of any one of embodiments 20-26, wherein the therapeutic anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.
28. The OBDS of any one of embodiments 1-19 and 24-27, or the needle syringe of any one of embodiments 20-27, wherein the heavy chain variable region comprises an N-terminal pyroglutamate residue.
29. A kit comprising the OBDS of any one of embodiments 1-19 and 24-28 or the needle syringe of any one of embodiments 20-28, and instructions for using the OBDS or the needle syringe for treatment of PNH or aHUS.
30. A method of treating a pediatric human patient with PNH or aHUS, wherein the patient is complement inhibitor treatment-naïve or eculizumab-experienced, the method comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
   (a) intravenously once on Day 1 of the administration cycle at a dose of:
      (i) 600 mg to a patient weighing ≥10 to <20 kg;
      (ii) 900 mg to a patient weighing ≥20 to <30 kg;
      (iii) 1200 mg to a patient weighing ≥30 to <40 kg;
      (iv) 2400 mg to a patient weighing ≥40 to <60 kg; or
      (v) 2700 mg to a patient weighing ≥60 kg, and
   (b) subcutaneously on Day 15 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
31. A method of treating a pediatric human patient with PNH or aHUS, wherein the patient is ravulizumab-experienced, the method comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
   subcutaneously on Day 1 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
32. The method of embodiment 30 or 31, wherein the pediatric human patient is less than 21 years of age.
33. The method of embodiment 32, wherein the pediatric human patient is between 2 and 18 years of age.
34. The method of any one of embodiments 30-33, wherein the therapeutic anti-C5 antibody, or antigen-binding fragment thereof, is administered subcutaneously to a patient weighing ≥10 to <20 kg at a dose of 150 mg using a syringe in a volume of 1.5 mL.
35. The method of any one of embodiments 30-34, wherein the 150 mg dose of the therapeutic anti-C5 antibody, or antigen-binding fragment thereof, is formulated at pH 7.4 and a concentration of 100 mg/mL in sodium phosphate, sucrose, L-arginine, PS80, and water for injection.
36. The method of any one of embodiments 30-33, wherein the therapeutic anti-C5 antibody, or antigen-binding fragment thereof, is administered subcutaneously to a patient weighing ≥20 kg using an OBDS.
37. The method of embodiment 36, wherein the OBDS comprises a cartridge comprising the therapeutic anti-C5 antibody, or antigen-binding fragment thereof, and an OBI.
38. The method of embodiment 36 or 37, wherein the therapeutic anti-C5 antibody, or antigen-binding fragment thereof, is administered subcutaneously to a patient weighing ≥20 to <40 kg at a dose of 245 mg using one OBDS.
39. The method of any one of embodiments 36-38, wherein the 245 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein the OBDS comprises one OBI and one pre-filled cartridge containing 245 mg of the therapeutic anti-C5 antibody or antigen-binding fragment thereof.
40. The method of embodiment 36 or 37, wherein the therapeutic anti-C5 antibody, or antigen-binding fragment thereof, is administered subcutaneously to a patient weighing ≥40 kg at a dose of 490 mg using two OBDSs.
41. The method of any one of embodiments 36, 37, and 40, wherein the 490 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein each OBDS comprises one OBI and one pre-filled cartridge containing 245 mg of the therapeutic anti-C5 antibody or antigen-binding fragment thereof.
42. The method of any one of embodiments 36-41, wherein the OBDS is a single-use electro-mechanical medical device comprising: an injector comprising a 29-gauge needle and a cartridge comprising a piston and telescopic screw assembly.
43. The method of any one of embodiments 37-42, wherein the cartridge has a volume of 3.5 mL.
44. The method of any one of embodiments 37-43, wherein the OBDS is configured to deliver the therapeutic anti-C5 antibody, or antigen-binding fragment thereof, via subcutaneous injection in about 10 minutes.
45. The method of any one of embodiments 37-41, wherein the cartridge comprises the therapeutic anti-C5 antibody, or antigen-binding fragment thereof, and an opening sealed by a pierceable septum.
46. The method of any one of embodiments 36-41, wherein the OBDS comprises an OBI comprising:
   (i) an injector housing;
   (ii) an injection needle translatable between a retracted position, wherein at least a tip of the injection needle is contained within the injector housing, and an injection position, wherein at least the tip of the injection needle protrudes from the injector housing;
   (iii) an activation button assembly movably mounted to the injector housing and operatively connected to the injection needle, the activation button assembly being translatable from an unactuated position to an actuated position to drive the injection needle from the retracted position thereof to the injection position thereof;
   (iv) a cartridge door movably mounted to the injector housing between an open position and a closed position, the cartridge door comprising:
      an open end,
      an interior channel having a cartridge mounted therein, the cartridge containing the therapeutic anti-C5 antibody, or antigen-binding fragment thereof, to be dispensed and having an opening at a front end of the cartridge sealed by a pierceable septum and a flange at a rear end thereof, and
      a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and
   (v) a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum;
   wherein the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum.
47. The method of embodiment 45 or 46, wherein the cartridge has a volume of 10 mL.
48. The method of any one of embodiments 30-47, wherein the therapeutic anti-C5 antibody, or antigen-binding fragment thereof, is self-administered subcutaneously to the patient.
49. The method of any one of embodiments 30-48, wherein the therapeutic anti-C5 antibody, or antigen-binding fragment thereof, is administered to a site selected from the patient's arm, abdomen, and thigh.
50. The method of any one of embodiments 30-49, wherein the therapeutic anti-C5 antibody, or antigen-binding fragment thereof, is administered subcutaneously once weekly after the administration cycle for up to 3 months, 6 months, 9 months, 12 months, 15 months, 18 months, 21 months, two years, or chronically for the remainder of the patient's life.
51. The method of any one of embodiments 30 and 34-50, wherein the patient has previously been treated with eculizumab or a biosimilar thereof or an anti-C5 antibody selected from tesidolumab, crovalimab, CAN106, or pozelimab.
52. The method of embodiment 51, wherein the patient has previously been treated with eculizumab or a biosimilar thereof.
53. The method of embodiment 51 or 52, wherein the administration cycle starts about two weeks after the patient's last dose of eculizumab or a biosimilar thereof or an anti-C5 antibody selected from tesidolumab, crovalimab, CAN106, or pozelimab.
54. The method of embodiment 53, wherein the administration cycle starts about two weeks after the patient's last dose of eculizumab or a biosimilar thereof.
55. The method of any one of embodiments 51-54, wherein the patient has been treated with eculizumab or a biosimilar thereof for at least 90 days prior to Day 1 of the administration cycle.
56. The method of any one of embodiments 31-55, wherein:
   (i) the patient weighs <20 kg, and the administration cycle starts about 4 weeks after the patient's last dose of ravulizumab; or
   (ii) the patient weighs ≥20 kg, and the administration cycle starts about 8 weeks after the patient's last dose of ravulizumab.
57. The method of any one of embodiments 30-56, wherein the therapeutic anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.
58. The method of any one of embodiments 30-57, wherein the therapeutic anti-C5 antibody, or antigen-binding fragment thereof, comprises a heavy chain variable region comprising the amino acid sequence of SEQ ID NO: 12 and a light chain variable region comprising the amino acid sequence of SEQ ID NO: 8.
59. The method of any one of embodiments 30-58, wherein the therapeutic anti-C5 antibody, or antigen-binding fragment thereof, further comprises a heavy chain constant region comprising the amino acid sequence of SEQ ID NO: 13.
60. The method of any one of embodiments 30-59, wherein the therapeutic anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.
61. The method of any one of embodiments 30-60, wherein the heavy chain variable region comprises an N-terminal pyroglutamate residue.
62. The method of any one of embodiments 30-61, wherein the therapeutic anti-C5 antibody, or antigen-binding fragment thereof, binds to human C5 at pH 7.4 and 25° C. with an affinity dissociation constant (K_{D}) that is in the range 0.1 nM≤K_{D}≤1 nM.
63. The method of any one of embodiments 30-62, wherein the therapeutic anti-C5 antibody, or antigen-binding fragment thereof, binds to human C5 at pH 6.0 and 25° C. with a K_{D} of ≥10 nM.
64. The method of any one of embodiments 30-63, wherein the therapeutic anti-C5 antibody is ravulizumab.
65. The method of any one of embodiments 30-64, wherein the patient is vaccinated against meningococcal infection from serogroups A, C, Y, W135, and B within 3 years prior to, or at least 2 weeks prior to, Day 1 of the administration cycle, and wherein the patient has received vaccination for Streptococcus pneumoniae and Haemophilus influenzae type b.
66. The method of any one of embodiments 30-65, wherein the patient has PNH.
67. The method of embodiment 66, wherein the patient has PNH confirmed by flow cytometry evaluation of red blood cells (RBCs) and white blood cells (WBCs), with a granulocyte or monocyte clone size of ≥5.
68. The method of embodiment 66 or 67, wherein the patient is complement inhibitor treatment-naïve, and has at least one or more of the following symptoms prior to treatment: fatigue, hemoglobinuria, abdominal pain, shortness of breath (dyspnea), anemia, history of a major adverse cardiovascular event (MAVE) (including thrombosis), dysphagia, or erectile dysfunction; or history of packed red blood cell transfusion due to PNH.
69. The method of any one of embodiments 66-68, wherein:
   (i) the patient is complement inhibitor treatment-naïve, and has lactate dehydrogenase (LDH) 1.5× upper limit of normal (ULN); or
   (ii) the patient is eculizumab- or ravulizumab-experienced, and has LDH ≤1.5×ULN.
70. The method of any one of any one of embodiments 66-69, wherein the treatment results in:
   (i) a change in LDH levels relative to baseline;
   (ii) a reduction in the incidence of breakthrough hemolysis;
   (iii) achievement of transfusion avoidance;
   (iv) achievement of stabilized hemoglobin; and/or
   (v) a change from baseline in PNH RBC clone size.
71. The method of embodiment 70, wherein the treatment results in:
   (i) a percentage change in LDH levels relative to baseline at Week 10 and Week 52;
   (ii) a reduction in the incidence of breakthrough hemolysis through Week 10 and Week 52;
   (iii) achievement of transfusion avoidance through Week 10 and Week 52;
   (iv) achievement of stabilized hemoglobin through Week 10 and Week 52; and
   (v) a change from baseline in PNH RBC clone size at Week 52.
72. The method of any one of embodiments 30-65, wherein the patient has aHUS.
73. The method of embodiment 72, wherein the treatment results in:
   (i) a change in dialysis requirement status;
   (ii) a change in observed value and change from baseline in estimated glomerular filtration rate (eGFR);
   (iii) a change in observed value and change from baseline in serum creatinine; and/or
   (iv) a change in observed value and change from baseline in hematologic parameters selected from (1) platelets; (2) lactate dehydrogenase (LDH); and/or (3) hemoglobin.
74. The method of embodiment 72 or 73, wherein the treatment results in:
   (i) a change in dialysis requirement status relative to baseline through Week 10 and Week 52;
   (ii) a change in observed value and change from baseline in eGFR through Week 10 and Week 52;
   (iii) a change in observed value and change from baseline in serum creatinine through Week 10 and Week 52; and/or
   (iv) a change in observed value and change from baseline in hematologic parameters selected from (1) platelets; (2) lactate dehydrogenase (LDH); and/or (3) hemoglobin through Week 10 and Week 52.
75. The method of any one of embodiments 72-74, wherein the patient is not administered an immunosuppressive therapy comprising a steroid, a mammalian target of rapamycin (mTOR) inhibitor, or a calcineurin inhibitor unless:
   (a) as part of an established post-transplant anti-rejection regimen;
   (b) the patient has confirmed anti-complement factor antibodies requiring immunosuppressive therapy;
   (c) the steroid is being used for treatment of a condition other than aHUS; or
   (d) the steroid was initiated empirically prior to treatment and is being tapered as standard of care.
76. The method of any one of embodiments 72-75, wherein the patient is complement inhibitor treatment-naïve and has evidence of thrombotic microangiopathy (TMA) based on the following prior to the administration cycle:
   (a) platelet count <150000/μL;
   (b) LDH ≥1.5× upper limit of normal (ULN);
   (c) hemoglobin lower limit of normal (LLN) for age and sex; and
   (d) serum creatinine level ≥97.5th percentile for age.
77. The method of any one of embodiments 72-75, wherein the patient is eculizumab-experienced or ravulizumab-experienced and had the following at the time of a TMA event:
   (a) increase in LDH >ULN;
   (b) increase in serum creatinine >ULN; and
   (c) decrease in platelets <LLN.
78. The method of any one of embodiments 72-75 and 77, wherein the patient is eculizumab-experienced or ravulizumab-experienced and had clinical evidence of response to eculizumab or ravulizumab indicated by stable TMA parameters comprising:
   (a) LDH <1.5×ULN;
   (b) platelet count ≥150000/μL; and
   (c) estimated glomerular filtration rate (eGFR) >30 mL/min/1.73 m² using the Schwartz formula.
79. The method of any one of embodiments 72-78, wherein the patient has a kidney transplant and has:
   (a) known history of aHUS prior to current kidney transplant; or
   (b) no known history of aHUS, and persistent evidence of TMA at least 4 days after modifying the immunosuppressive regimen of calcineurin inhibitor or mammalian target of rapamycin inhibitor.
80. The method of any one of embodiments 72-79, wherein the patient had onset of TMA postpartum, and the patient has persistent symptoms of TMA for greater than 3 days after the day of childbirth.
81. The method of any one of any one of embodiments 72-80, wherein the treatment results in:
   (i) an improvement in the patient's dialysis requirement status;
   (ii) a change in eGFR levels relative to baseline;
   (iii) a change in serum creatinine levels relative to baseline;
   (iv) a change in hematologic parameters comprising platelets, LDH, and/or hemoglobin levels relative to baseline.
82. The method of any one of embodiments 30-81, wherein the treatment maintains (a) a serum trough concentration of free C5 of <0.5 μg/mL during the administration cycle and/or (b) a serum concentration of 175 μg/mL of the therapeutic anti-C5 antibody, or antigen-binding fragment thereof, during the administration cycle.
83. The method of any one of embodiments 30-82, wherein the method further comprises monitoring the concentration of:
   (a) serum anti-C5 antibody, or antigen-binding fragment thereof, and/or
   (b) serum free C5.
84. The method of embodiment 83, wherein the concentration of:
   (a) serum anti-C5 antibody, or antigen-binding fragment thereof, and/or
   (b) serum free C5,
   is determined from blood samples collected before and after each administration of the therapeutic anti-C5 antibody, or antigen-binding fragment thereof.
85. The method of embodiment 84, wherein the blood samples are collected within 30 minutes prior to the administration and within 60 minutes after each administration of the therapeutic anti-C5 antibody, or antigen-binding fragment thereof.
86. The method of any one of any one of embodiments 30-85, wherein the treatment results in terminal complement inhibition.
87. The method of any one of any one of embodiments 30-86, wherein the treatment results in a reduction of hemolysis as assessed by LDH levels.
88. The method of any one of any one of embodiments 30-87, wherein the method comprises monitoring:
   (a) the incidence of (i) adverse events (AEs) and serious AEs and (ii) adverse drug events (ADEs) and serious ADEs;
   (b) the outcome of attempted full-dose administration via an OBI and/or reported device deficiencies or complaints and associated device investigations; and/or
   (c) the incidence of anti-drug antibodies (ADAs), response categories, and titer.
89. The method of any one of embodiments 30-88, wherein the treatment produces a change from baseline in patient-reported fatigue, as measured by Pediatric FACIT-Fatigue, optionally wherein the patient is 8 years of age.
90. The method of any one of embodiments 30-89, wherein the treatment produces a change from baseline in PedsQL 4.0 Generic Core Scale.
91. A kit for treating PNH or aHUS in a pediatric human patient, wherein the patient is complement inhibitor treatment-naïve or eculizumab-experienced, comprising:
   (a) a dose of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively; and
(b) instructions for using the therapeutic anti-C5 antibody, or antigen-binding fragment thereof, in the method of any one of embodiments 30 and 32-89.
92. The kit of embodiment 91, wherein the therapeutic anti-C5 antibody, or antigen-binding fragment thereof, is administered:
   (a) intravenously once on Day 1 of the administration cycle at a dose of:
      (i) 600 mg to a patient weighing ≥10 to <20 kg;
      (ii) 900 mg to a patient weighing ≥20 to <30 kg;
      (iii) 1200 mg to a patient weighing ≥30 to <40 kg;
      (iv) 2400 mg to a patient weighing ≥40 to <60 kg; or
      (v) 2700 mg to a patient weighing ≥60 kg, and
   (b) subcutaneously on Day 15 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
93. The kit of embodiment 91 or 92, wherein the therapeutic anti-C5 antibody, or antigen-binding fragment thereof, is provided in a 3 mL vial comprising Type I borosilicate glass for (a) intravenous administration and (b) subcutaneous administration to a patient weighing ≥10 to <20 kg.
94. A kit for treating PNH or aHUS in a pediatric human patient, wherein the patient is ravulizumab-experienced, comprising:
   (a) a dose of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively; and
   (b) instructions for using the therapeutic anti-C5 antibody, or antigen-binding fragment thereof, in the method of any one of embodiments 31-89.
95. The kit of embodiment 94, wherein the therapeutic anti-C5 antibody, or antigen-binding fragment thereof, is administered:
   subcutaneously on Day 1 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
96. The kit of any one of embodiments 91-95, wherein the pediatric human patient is less than 21 years of age.
97. The kit of embodiment 96, wherein the pediatric human patient is between 2 and 18 years of age.
98. The kit of any one of embodiments 91-97, wherein the therapeutic anti-C5 antibody, or antigen-binding fragment thereof, is administered to a site selected from the patient's arm, abdomen, and thigh.
99. A therapeutic anti-C5 antibody, or antigen-binding fragment thereof, for use in treatment of a pediatric human patient with PNH or aHUS, wherein the patient is complement inhibitor treatment-naïve or eculizumab-experienced, the treatment comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
   (a) intravenously once on Day 1 of the administration cycle at a dose of:
      (i) 600 mg to a patient weighing ≥10 to <20 kg;
      (ii) 900 mg to a patient weighing ≥20 to <30 kg;
      (iii) 1200 mg to a patient weighing ≥30 to <40 kg;
      (iv) 2400 mg to a patient weighing ≥40 to <60 kg; or
      (v) 2700 mg to a patient weighting ≥60 kg, and
   (b) subcutaneously on Day 15 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
100. Use of a therapeutic anti-C5 antibody, or antigen-binding fragment thereof, in the manufacture of a medicament for treating aHUS or PNH in a pediatric human patient in need thereof, wherein the therapeutic anti-C5 antibody, or antigen-binding fragment thereof comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is complement inhibitor treatment-naïve or eculizumab-experienced, and wherein the therapeutic anti-C5 antibody, or antigen-binding fragment thereof, is formulated for administration by:
   (a) intravenously once on Day 1 of the administration cycle at a dose of:
      (i) 600 mg to a patient weighing ≥10 to <20 kg;
      (ii) 900 mg to a patient weighing ≥20 to <30 kg;
      (iii) 1200 mg to a patient weighing ≥30 to <40 kg;
      (iv) 2400 mg to a patient weighing ≥40 to <60 kg; or
      (v) 2700 mg to a patient weighting ≥60 kg, and
   (b) subcutaneously on Day 15 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
101. A therapeutic anti-C5 antibody, or antigen-binding fragment thereof, for use in treatment of a pediatric human patient with PNH or aHUS, wherein the patient is ravulizumab-experienced, the treatment comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
   subcutaneously on Day 1 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg.
102. Use of a therapeutic anti-C5 antibody, or antigen-binding fragment thereof, in the manufacture of a medicament for treating aHUS or PNH in a pediatric human patient in need thereof, wherein the therapeutic anti-C5 antibody, or antigen-binding fragment thereof, comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ravulizumab-experienced, and wherein the therapeutic anti-C5 antibody, or antigen-binding fragment thereof, is formulated for administration:
   subcutaneously on Day 1 of the administration cycle and every week thereafter at a dose of:
      (i) 150 mg to a patient weighing ≥10 to <20 kg;
      (ii) 245 mg to a patient weighing ≥20 to <40 kg; or
      (iii) 490 mg to a patient weighing ≥40 kg once per week.
103. The therapeutic anti-C5 antibody, or antigen-binding fragment thereof, of embodiment 99 or 101, or the use of embodiment 100 or 102, wherein the pediatric human patient is less than 21 years of age.
104. The therapeutic anti-C5 antibody, or antigen-binding fragment thereof, of any one of embodiments 99, 101, and 103, or the use of any one of embodiments 100, 102, and 103, wherein the pediatric human patient is between 2 and 18 years of age.
105. The therapeutic anti-C5 antibody, or antigen-binding fragment thereof, of any one of embodiments 99, 101, and 103-104, or the use of any one of embodiments 100 and 102-104, configured for subcutaneous administration to a site selected from the patient's arm, abdomen, and thigh.

### Sequence Summary

The polypeptide sequence of ravulizumab is available in the KEGG DRUG database under entry number D1105, in Chemical Abstracts under accession number CAS 1803171-55-2, and in PubChem under accession number 363669767.

The polypeptide sequence of ravulizumab, as accessioned in KEGG DRUG Database provides that the N-terminal amino acid of variable heavy chain is “X”. The Chemical Abstracts (CAS) for ravulizumab (CAS 1803171-55-2) also provides that N-terminal X is pyroglutamic acid (designated as “chain 1 pyroglutamic acid-1” in the CAS report). Although this information may seem different from the VH sequence of ravulizumab, e.g., a heavy chain variable region polypeptide comprising the amino acid sequence depicted in SEQ ID NO: 12 and/or a heavy chain polypeptide comprising the amino acid sequence depicted in SEQ ID NO: 14, there is alignment between patented sequences and the drug database/CAS sequences because it was recognized that N-terminal Q in polypeptide and/or antibody sequence cyclizes during process development to yield drug product conversion to pyroglutamate (pyro-Q) near 100%, as disclosed in Liu et al. (J Pharm Sci. 2019 October; 108(10):3194-3200) and Nguyen et al. (Int J Mol Sci. 2017 Jul. 20; 18(7):1575).

Additional information is provided in page 7 and Table 4 of Xu et al. (MAbs, 2019 February/March; 11(2):239-264) and these referenced publications: (1) Yu et al. Investigation of N-terminal glutamate cyclization of recombinant monoclonal antibody in formulation development. J. Pharm. Biomed. Anal., 2006, 42, 455-463 and Dick et al. “Determination of the origin of the N-terminal pyro-glutamate variation in monoclonal antibodies using model peptides.” Biotechnol. Bioeng., 2007, 97, 544-553, the disclosures in which are incorporated by reference in their entirety.

SEQUENCE SUMMARY TABLE

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSS
SEQ ID NO: 8
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK
SEQ ID NO: 9
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
SEQ ID NO: 10
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 12
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
SEQ ID NO: 16
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVTSSNFGT
QTYTCNVDHKPSNTKVDKTVERKCCVECPPCPAPPVAGPSVFLFPPKPKD
TLYITREPEVTCVVVDVSHEDPEVQFNWYVDGMEVHNAKTKPREEQFNST
FRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSWVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGG
IGPFFGTANYAQKFQGRVTITADESTSTAYMELSSLRSEDTAVYYCARDT
PYFDYWGQGTLVTVSS
SEQ ID NO: 28
DIELTQPPSVSVAPGQTARISCSGDSIPNYYVYWYQQKPGQAPVLVIYDD
SNRPSGIPERFSGSNSGNTATLTISGTQAEDEADYYCQSFDSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGII
DPDDSYTEYSPSFQGQVTISADKSISTAYLQWSSLKASDTAMYYCARYEY
GGFDIWGQGTLVTVSS
SEQ ID NO: 36
SYELTQPPSVSVAPGQTARISCSGDNIGNSYVHWYQQKPGQAPVLVIYKD
NDRPSGIPERFSGSNSGNTATLTISGTQAEDEADYYCGTYDIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVG
AIYTGSGATYKASWAKGRFTISKDTSKNQVVLTMTNMDPVDTATYYCASD
GGYDYPTHAMHYWGQGTLVTVSS
SEQ ID NO: 44
DWVMTQSPSSLSASVGDRVTITCQASQNIGSSLAWYQQKPGQAPRLLIYG
ASKTHSGVPSRFSGSGSGTDFTLTISSLQPEDVATYYCQSTKVGSSYGNH
FGGGTKVEIK
SEQ ID NO: 45
QVQLVESGGGLVQPGRSLRLSCAASGFTVHSSYYMAWVRQAPGKGLEWVG
AIFTGSGAEYKAEWAKGRVTISKDTSKNQVVLTMTNMDPVDTATYYCASD
AGYDYPTHAMHYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELRRGPKVFLFP
PKPKDTLMISRTPEVTCVVVDVSHEDPEVKFNWYVDGVEVHNAKTKPREE
QYNSTYRVVSVLTVLHQDWLNGKEYKCKVSNKGLPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVLHEALHAHYTRKELSLS
P
SEQ ID NO: 46
DIQMTQSPSSLSASVGDRVTITCRASQGISSSLAWYQQKPGKAPKLLIYG
ASETESGVPSRFSGSGSGTDFTLTISSLQPEDFATYYCQNTKVGSSYGNT
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
SEQ ID NO: 47
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSS
SEQ ID NO: 48
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIK
SEQ ID NO: 49
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTQNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

What is claimed is:

1. An on-body delivery system (OBDS) configured for administration of an effective amount of a therapeutic anti-C5 antibody, or antigen-binding fragment thereof, comprising complementarity determining region (CDR) 1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat atypical hemolytic uremic syndrome (aHUS) or paroxysmal nocturnal hemoglobinuria (PNH) in a pediatric human patient in need thereof, wherein the patient is complement inhibitor treatment-naïve or eculizumab-experienced, wherein the therapeutic anti-C5 antibody, or antigen-binding fragment thereof, is administered:
subcutaneously once per week at a dose of:
(i) 245 mg to a patient weighing ≥20 to <40 kg; or
(ii) 490 mg to a patient weighing ≥40 kg.

2. An OBDS configured for treating aHUS or PNH in a pediatric human patient in need thereof, wherein the OBDS comprises an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is complement inhibitor treatment-naïve or eculizumab-experienced, and wherein the OBDS comprises means for subcutaneous administration of:
(i) 245 mg to a patient weighing ≥20 to <40 kg; or
(ii) 490 mg to a patient weighing ≥40 kg.

3. An OBDS configured for administration of an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat aHUS or PNH in a pediatric human patient in need thereof, wherein the patient is ravulizumab-experienced, wherein the therapeutic anti-C5 antibody, or antigen-binding fragment thereof, is administered:
subcutaneously once per week at a dose of:
(i) 245 mg to a patient weighing ≥20 to <40 kg; or
(ii) 490 mg to a patient weighing ≥40 kg.

4. An OBDS configured for treating aHUS or PNH in a pediatric human patient in need thereof, wherein the OBDS comprises an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ravulizumab-experienced, and wherein the OBDS comprises means for subcutaneous administration of:
(i) 245 mg to a patient weighing ≥20 to <40 kg; or
(ii) 490 mg to a patient weighing ≥40 kg.

5. The OBDS of any one of claims 1-4, wherein the pediatric human patient is less than 21 years of age.

6. The OBDS of claim 5, wherein the pediatric human patient is between 2 and 18 years of age.

7. The OBDS of any one of claims 1-6, wherein the OBDS comprises a cartridge comprising the therapeutic anti-C5 antibody, or antigen-binding fragment thereof, and an on-body injector (OBI).

8. The OBDS of any one of claims 1-7, wherein the OBDS is configured for administration of the therapeutic anti-C5 antibody, or antigen-binding fragment thereof, subcutaneously to a patient weighing ≥20 to <40 kg at a dose of 245 mg using one OBDS.

9. The OBDS of claim 8, wherein the 245 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% weight by volume (w/v) sucrose, 0.05% w/v polysorbate 80 (PS80), and water for injection, and wherein the OBDS comprises one OBI and one pre-filled cartridge containing 245 mg of the therapeutic anti-C5 antibody or antigen-binding fragment thereof.

10. The OBDS of any one of claims 1-7, wherein the OBDS is configured for administration of the therapeutic anti-C5 antibody, or antigen-binding fragment thereof, subcutaneously to a patient weighing ≥40 kg at a dose of 490 mg in combination with a second OBDS.

11. The OBDS of claim 10, wherein the 490 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein each OBDS comprises one OBI and one pre-filled cartridge containing 245 mg of the therapeutic anti-C5 antibody or antigen-binding fragment thereof.

12. The OBDS of any one of claims 1-11, wherein the OBDS is a single-use electro-mechanical medical device comprising: an injector comprising a 29-gauge needle and a cartridge comprising a piston and telescopic screw assembly.

13. The OBDS of any one of claims 7-12, wherein the cartridge has a volume of 3.5 mL.

14. The OBDS of any one of claims 7-13, wherein the OBDS is configured to deliver the therapeutic anti-C5 antibody, or antigen-binding fragment thereof, via subcutaneous injection in about 10 minutes.

15. The OBDS of any one of claims 7-14, wherein the cartridge comprises the therapeutic anti-C5 antibody, or antigen-binding fragment thereof, and an opening sealed by a pierceable septum.

16. The OBDS of any one of claims 1-15, wherein the OBDS comprises an OBI comprising:
(i) an injector housing;
(ii) an injection needle translatable between a retracted position, wherein at least a tip of the injection needle is contained within the injector housing, and an injection position, wherein at least the tip of the injection needle protrudes from the injector housing;
(iii) an activation button assembly movably mounted to the injector housing and operatively connected to the injection needle, the activation button assembly being translatable from an unactuated position to an actuated position to drive the injection needle from the retracted position thereof to the injection position thereof;
(iv) a cartridge door movably mounted to the injector housing between an open position and a closed position, the cartridge door comprising:
an open end,
an interior channel having a cartridge mounted therein, the cartridge containing the therapeutic anti-C5 antibody, or antigen-binding fragment thereof, to be dispensed and having an opening at a front end of the cartridge sealed by a pierceable septum and a flange at a rear end thereof, and
a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and
(v) a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum;
wherein the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum.

17. The OBDS of claim 15 or 16, wherein the cartridge has a volume of 10 mL.

18. The OBDS of any one of claims 1-17, wherein the OBDS is configured for self-administration of the therapeutic anti-C5 antibody, or antigen-binding fragment thereof, subcutaneously to the patient.

19. The OBDS of any one of claims 1-18, wherein the OBDS is configured for subcutaneous administration of the therapeutic anti-C5 antibody, or antigen-binding fragment thereof, to a site selected from the patient's arm, abdomen, and thigh.

20. A needle syringe configured for administration of an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, to treat aHUS or PNH in a pediatric human patient in need thereof, wherein the patient is ≥10 to <20 kg of body weight and is complement inhibitor treatment-naïve, eculizumab-experienced, or ravulizumab-experienced, wherein the therapeutic anti-C5 antibody, or antigen-binding fragment thereof, is administered: subcutaneously once per week at a dose of 150 mg.

21. A needle syringe configured for treating aHUS or PNH in a pediatric human patient in need thereof, wherein the needle syringe is configured for subcutaneous administration and comprises 150 mg of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ≥10 to <20 kg of body weight and is complement inhibitor treatment-naïve, eculizumab-experienced, or ravulizumab-experienced.

22. The needle syringe of claim 20 or 21, wherein the 150 mg dose of the therapeutic anti-C5 antibody, or antigen-binding fragment thereof, is formulated at pH 7.4 and a concentration of 100 mg/mL in sodium phosphate, sucrose, L-arginine, PS80, and water for injection.

23. The needle syringe of any one of claims 20-22, wherein the needle syringe is configured for administration of the therapeutic anti-C5 antibody, or antigen-binding fragment thereof, to a site selected from the patient's arm, abdomen, and thigh.

24. The OBDS of any one of claims 1-19, or the needle syringe of any one of claims 20-23, wherein the therapeutic anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

25. The OBDS of any one of claims 1-19 and 24, or the needle syringe of any one of claims 20-23 and 24, wherein the therapeutic anti-C5 antibody, or antigen-binding fragment thereof, comprises a heavy chain variable region comprising the amino acid sequence of SEQ ID NO: 12 and a light chain variable region comprising the amino acid sequence of SEQ ID NO: 8.

26. The OBDS of any one of claims 1-19, 24, and 25, or the needle syringe of any one of claims 20-25, wherein the therapeutic anti-C5 antibody, or antigen-binding fragment thereof, further comprises a heavy chain constant region comprising the amino acid sequence of SEQ ID NO: 13.

27. The OBDs of any one of claims 1-19 and 24-26, or the needle syringe of any one of claims 20-26, wherein the therapeutic anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.

28. The OBDS of any one of claims 1-19 and 24-27, or the needle syringe of any one of claims 20-27, wherein the heavy chain variable region comprises an N-terminal pyroglutamate residue.

29. A kit comprising the OBDS of any one of claims 1-19 and 24-28 or the needle syringe of any one of claims 20-28, and instructions for using the OBDS or the needle syringe for treatment of PNH or aHUS.

30. A method of treating a pediatric human patient with PNH or aHUS, wherein the patient is complement inhibitor treatment-naïve or eculizumab-experienced, the method comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
(a) intravenously once on Day 1 of the administration cycle at a dose of:
(i) 600 mg to a patient weighing ≥10 to <20 kg;
(ii) 900 mg to a patient weighing ≥20 to <30 kg;
(iii) 1200 mg to a patient weighing ≥30 to <40 kg;
(iv) 2400 mg to a patient weighing ≥40 to <60 kg; or
(v) 2700 mg to a patient weighing ≥60 kg, and
(b) subcutaneously on Day 15 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

31. A method of treating a pediatric human patient with PNH or aHUS, wherein the patient is ravulizumab-experienced, the method comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

32. The method of claim 30 or 31, wherein the pediatric human patient is less than 21 years of age.

33. The method of claim 32, wherein the pediatric human patient is between 2 and 18 years of age.

34. The method of any one of claims 30-33, wherein the therapeutic anti-C5 antibody, or antigen-binding fragment thereof, is administered subcutaneously to a patient weighing ≥10 to <20 kg at a dose of 150 mg using a syringe in a volume of 1.5 mL.

35. The method of any one of claims 30-34, wherein the 150 mg dose of the therapeutic anti-C5 antibody, or antigen-binding fragment thereof, is formulated at pH 7.4 and a concentration of 100 mg/mL in sodium phosphate, sucrose, L-arginine, PS80, and water for injection.

36. The method of any one of claims 30-33, wherein the therapeutic anti-C5 antibody, or antigen-binding fragment thereof, is administered subcutaneously to a patient weighing ≥20 kg using an OBDS.

37. The method of claim 36, wherein the OBDS comprises a cartridge comprising the therapeutic anti-C5 antibody, or antigen-binding fragment thereof, and an OBI.

38. The method of claim 36 or 37, wherein the therapeutic anti-C5 antibody, or antigen-binding fragment thereof, is administered subcutaneously to a patient weighing ≥20 to <40 kg at a dose of 245 mg using one OBDS.

39. The method of any one of claims 36-38, wherein the 245 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein the OBDS comprises one OBI and one pre-filled cartridge containing 245 mg of the therapeutic anti-C5 antibody or antigen-binding fragment thereof.

40. The method of claim 36 or 37, wherein the therapeutic anti-C5 antibody, or antigen-binding fragment thereof, is administered subcutaneously to a patient weighing ≥40 kg at a dose of 490 mg using two OBDSs.

41. The method of any one of claims 36, 37, and 40, wherein the 490 mg dose of the therapeutic anti-C5 antibody, or antigen-binding fragment thereof, is formulated at pH 7.4 and a concentration of 70 mg/mL in 50 mM sodium phosphate, 25 mM L-arginine, 5% w/v sucrose, 0.05% w/v PS80, and water for injection, and wherein each OBDS comprises one OBI and one pre-filled cartridge containing 245 mg of the therapeutic anti-C5 antibody or antigen-binding fragment thereof.

42. The method of any one of claims 36-41, wherein the OBDS is a single-use electro-mechanical medical device comprising: an injector comprising a 29-gauge needle and a cartridge comprising a piston and telescopic screw assembly.

43. The method of any one of claims 37-42, wherein the cartridge has a volume of 3.5 mL.

44. The method of any one of claims 37-43, wherein the OBDS is configured to deliver the therapeutic anti-C5 antibody, or antigen-binding fragment thereof, via subcutaneous injection in about 10 minutes.

45. The method of any one of claims 37-41, wherein the cartridge comprises the therapeutic anti-C5 antibody, or antigen-binding fragment thereof, and an opening sealed by a pierceable septum.

46. The method of any one of claims 36-41, wherein the OBDS comprises an OBI comprising:
(i) an injector housing;
(ii) an injection needle translatable between a retracted position, wherein at least a tip of the injection needle is contained within the injector housing, and an injection position, wherein at least the tip of the injection needle protrudes from the injector housing;
(iii) an activation button assembly movably mounted to the injector housing and operatively connected to the injection needle, the activation button assembly being translatable from an unactuated position to an actuated position to drive the injection needle from the retracted position thereof to the injection position thereof;
(iv) a cartridge door movably mounted to the injector housing between an open position and a closed position, the cartridge door comprising:
an open end,
an interior channel having a cartridge mounted therein, the cartridge containing the therapeutic anti-C5 antibody, or antigen-binding fragment thereof, to be dispensed and having an opening at a front end of the cartridge sealed by a pierceable septum and a flange at a rear end thereof, and
a cartridge piercing needle mounted within the interior channel and connected in fluid communication with the injection needle, the cartridge piercing needle being configured to fully penetrate the pierceable septum of the cartridge to connect the substance within the cartridge in fluid communication with the injection needle; and
(v) a deflectable interference member engaging the rear end flange of the cartridge in a resting position of the interference member, thereby limiting an insertion depth of the cartridge into the interior channel of the cartridge door to a sealed position, wherein the cartridge piercing needle does not fully penetrate the pierceable septum;
wherein the cartridge door is movable to the closed position thereof in the sealed position of the cartridge, and movement of the activation button assembly from the unactuated position to the actuated position thereof, in the closed position of the cartridge door, deflects the interference element out of engagement with the rear end flange of the cartridge, thereby enabling further advancement of the cartridge into the interior channel of the cartridge door to an unsealed position, wherein the cartridge piercing needle fully penetrates the pierceable septum.

47. The method of claim 45 or 46, wherein the cartridge has a volume of 10 mL.

48. The method of any one of claims 30-47, wherein the therapeutic anti-C5 antibody, or antigen-binding fragment thereof, is self-administered subcutaneously to the patient.

49. The method of any one of claims 30-48, wherein the therapeutic anti-C5 antibody, or antigen-binding fragment thereof, is administered to a site selected from the patient's arm, abdomen, and thigh.

50. The method of any one of claims 30-49, wherein the therapeutic anti-C5 antibody, or antigen-binding fragment thereof, is administered subcutaneously once weekly after the administration cycle for up to 3 months, 6 months, 9 months, 12 months, 15 months, 18 months, 21 months, two years, or chronically for the remainder of the patient's life.

51. The method of any one of claims 30 and 34-50, wherein the patient has previously been treated with eculizumab or a biosimilar thereof or an anti-C5 antibody selected from tesidolumab, crovalimab, CAN106, or pozelimab.

52. The method of claim 51, wherein the patient has previously been treated with eculizumab or a biosimilar thereof.

53. The method of claim 51 or 52, wherein the administration cycle starts about two weeks after the patient's last dose of eculizumab or a biosimilar thereof or an anti-C5 antibody selected from tesidolumab, crovalimab, CAN106, or pozelimab.

54. The method of claim 53, wherein the administration cycle starts about two weeks after the patient's last dose of eculizumab or a biosimilar thereof.

55. The method of any one of claims 51-54, wherein the patient has been treated with eculizumab or a biosimilar thereof for at least 90 days prior to Day 1 of the administration cycle.

56. The method of any one of claims 31-55, wherein:
(i) the patient weighs <20 kg, and the administration cycle starts about 4 weeks after the patient's last dose of ravulizumab; or
(ii) the patient weighs 20 kg, and the administration cycle starts about 8 weeks after the patient's last dose of ravulizumab.

57. The method of any one of claims 30-56, wherein the therapeutic anti-C5 antibody, or antigen-binding fragment thereof, comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

58. The method of any one of claims 30-57, wherein the therapeutic anti-C5 antibody, or antigen-binding fragment thereof, comprises a heavy chain variable region comprising the amino acid sequence of SEQ ID NO: 12 and a light chain variable region comprising the amino acid sequence of SEQ ID NO: 8.

59. The method of any one of claims 30-58, wherein the therapeutic anti-C5 antibody, or antigen-binding fragment thereof, further comprises a heavy chain constant region comprising the amino acid sequence of SEQ ID NO: 13.

60. The method of any one of claims 30-59, wherein the therapeutic anti-C5 antibody, or antigen-binding fragment thereof, comprises a heavy chain polypeptide comprising the amino acid sequence of SEQ ID NO: 14 and a light chain polypeptide comprising the amino acid sequence of SEQ ID NO: 11.

61. The method of any one of claims 30-60, wherein the heavy chain variable region comprises an N-terminal pyroglutamate residue.

62. The method of any one of claims 30-61, wherein the therapeutic anti-C5 antibody, or antigen-binding fragment thereof, binds to human C5 at pH 7.4 and 25° C. with an affinity dissociation constant (K_{D}) that is in the range 0.1 nM≤K_{D}≤1 nM.

63. The method of any one of claims 30-62, wherein the therapeutic anti-C5 antibody, or antigen-binding fragment thereof, binds to human C5 at pH 6.0 and 25° C. with a K_{D} of ≥10 nM.

64. The method of any one of claims 30-63, wherein the therapeutic anti-C5 antibody is ravulizumab.

65. The method of any one of claims 30-64, wherein the patient is vaccinated against meningococcal infection from serogroups A, C, Y, W135, and B within 3 years prior to, or at least 2 weeks prior to, Day 1 of the administration cycle, and wherein the patient has received vaccination for Streptococcus pneumoniae and Haemophilus influenzae type b.

66. The method of any one of claims 30-65, wherein the patient has PNH.

67. The method of claim 66, wherein the patient has PNH confirmed by flow cytometry evaluation of red blood cells (RBCs) and white blood cells (WBCs), with a granulocyte or monocyte clone size of 5.

68. The method of claim 66 or 67, wherein the patient is complement inhibitor treatment-naïve, and has at least one or more of the following symptoms prior to treatment: fatigue, hemoglobinuria, abdominal pain, shortness of breath (dyspnea), anemia, history of a major adverse cardiovascular event (MAVE) (including thrombosis), dysphagia, or erectile dysfunction; or history of packed red blood cell transfusion due to PNH.

69. The method of any one of claims 66-68, wherein:
(i) the patient is complement inhibitor treatment-naïve, and has lactate dehydrogenase (LDH) ≥1.5× upper limit of normal (ULN); or
(ii) the patient is eculizumab- or ravulizumab-experienced, and has LDH ≤1.5×ULN.

70. The method of any one of any one of claims 66-69, wherein the treatment results in:
(i) a change in LDH levels relative to baseline;
(ii) a reduction in the incidence of breakthrough hemolysis;
(iii) achievement of transfusion avoidance;
(iv) achievement of stabilized hemoglobin; and/or
(v) a change from baseline in PNH RBC clone size.

71. The method of claim 70, wherein the treatment results in:
(i) a percentage change in LDH levels relative to baseline at Week 10 and Week 52;
(ii) a reduction in the incidence of breakthrough hemolysis through Week 10 and Week 52;
(iii) achievement of transfusion avoidance through Week 10 and Week 52;
(iv) achievement of stabilized hemoglobin through Week 10 and Week 52; and
(v) a change from baseline in PNH RBC clone size at Week 52.

72. The method of any one of claims 30-65, wherein the patient has aHUS.

73. The method of claim 72, wherein the treatment results in:
(i) a change in dialysis requirement status;
(ii) a change in observed value and change from baseline in estimated glomerular filtration rate (eGFR);
(iii) a change in observed value and change from baseline in serum creatinine; and/or
(iv) a change in observed value and change from baseline in hematologic parameters selected from (1) platelets; (2) lactate dehydrogenase (LDH); and/or (3) hemoglobin.

74. The method of claim 72 or 73, wherein the treatment results in:
(i) a change in dialysis requirement status relative to baseline through Week 10 and Week 52;
(ii) a change in observed value and change from baseline in eGFR through Week 10 and Week 52;
(iii) a change in observed value and change from baseline in serum creatinine through Week 10 and Week 52; and/or
(iv) a change in observed value and change from baseline in hematologic parameters selected from (1) platelets; (2) lactate dehydrogenase (LDH); and/or (3) hemoglobin through Week 10 and Week 52.

75. The method of any one of claims 72-74, wherein the patient is not administered an immunosuppressive therapy comprising a steroid, a mammalian target of rapamycin (mTOR) inhibitor, or a calcineurin inhibitor unless:
(a) as part of an established post-transplant anti-rejection regimen;
(b) the patient has confirmed anti-complement factor antibodies requiring immunosuppressive therapy;
(c) the steroid is being used for treatment of a condition other than aHUS; or
(d) the steroid was initiated empirically prior to treatment and is being tapered as standard of care.

76. The method of any one of claims 72-75, wherein the patient is complement inhibitor treatment-naïve and has evidence of thrombotic microangiopathy (TMA) based on the following prior to the administration cycle:
(a) platelet count <150000/μL;
(b) LDH ≥1.5× upper limit of normal (ULN);
(c) hemoglobin ≤lower limit of normal (LLN) for age and sex; and
(d) serum creatinine level 97.5th percentile for age.

77. The method of any one of claims 72-75, wherein the patient is eculizumab-experienced or ravulizumab-experienced and had the following at the time of a TMA event:
(a) increase in LDH >ULN;
(b) increase in serum creatinine >ULN; and
(c) decrease in platelets <LLN.

78. The method of any one of claims 72-75 and 77, wherein the patient is eculizumab-experienced or ravulizumab-experienced and had clinical evidence of response to eculizumab or ravulizumab indicated by stable TMA parameters comprising:
(a) LDH <1.5×ULN;
(b) platelet count 150000/μL; and
(c) estimated glomerular filtration rate (eGFR) >30 mL/min/1.73 m² using the Schwartz formula.

79. The method of any one of claims 72-78, wherein the patient has a kidney transplant and has:
(a) known history of aHUS prior to current kidney transplant; or
(b) no known history of aHUS, and persistent evidence of TMA at least 4 days after modifying the immunosuppressive regimen of calcineurin inhibitor or mammalian target of rapamycin inhibitor.

80. The method of any one of claims 72-79, wherein the patient had onset of TMA postpartum, and the patient has persistent symptoms of TMA for greater than 3 days after the day of childbirth.

81. The method of any one of any one of claims 72-80, wherein the treatment results in:
(i) an improvement in the patient's dialysis requirement status;
(ii) a change in eGFR levels relative to baseline;
(iii) a change in serum creatinine levels relative to baseline;
(iv) a change in hematologic parameters comprising platelets, LDH, and/or hemoglobin levels relative to baseline.

82. The method of any one of claims 30-81, wherein the treatment maintains (a) a serum trough concentration of free C5 of <0.5 μg/mL during the administration cycle and/or (b) a serum concentration of 175 μg/mL of the therapeutic anti-C5 antibody, or antigen-binding fragment thereof, during the administration cycle.

83. The method of any one of claims 30-82, wherein the method further comprises monitoring the concentration of:
(a) serum anti-C5 antibody, or antigen-binding fragment thereof, and/or
(b) serum free C5.

84. The method of claim 83, wherein the concentration of:
(a) serum anti-C5 antibody, or antigen-binding fragment thereof, and/or
(b) serum free C5,
is determined from blood samples collected before and after each administration of the therapeutic anti-C5 antibody, or antigen-binding fragment thereof.

85. The method of claim 84, wherein the blood samples are collected within 30 minutes prior to the administration and within 60 minutes after each administration of the therapeutic anti-C5 antibody, or antigen-binding fragment thereof.

86. The method of any one of any one of claims 30-85, wherein the treatment results in terminal complement inhibition.

87. The method of any one of any one of claims 30-86, wherein the treatment results in a reduction of hemolysis as assessed by LDH levels.

88. The method of any one of any one of claims 30-87, wherein the method comprises monitoring:
(a) the incidence of (i) adverse events (AEs) and serious AEs and (ii) adverse drug events (ADEs) and serious ADEs;
(b) the outcome of attempted full-dose administration via an OBI and/or reported device deficiencies or complaints and associated device investigations; and/or
(c) the incidence of anti-drug antibodies (ADAs), response categories, and titer.

89. The method of any one of claims 30-88, wherein the treatment produces a change from baseline in patient-reported fatigue, as measured by Pediatric FACIT-Fatigue, optionally wherein the patient is ≥8 years of age.

90. The method of any one of claims 30-89, wherein the treatment produces a change from baseline in PedsQL 4.0 Generic Core Scale.

91. A kit for treating PNH or aHUS in a pediatric human patient, wherein the patient is complement inhibitor treatment-naïve or eculizumab-experienced, comprising:
(a) a dose of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively; and
(b) instructions for using the therapeutic anti-C5 antibody, or antigen-binding fragment thereof, in the method of any one of claims 30 and 32-89.

92. The kit of claim 91, wherein the therapeutic anti-C5 antibody, or antigen-binding fragment thereof, is administered:
(a) intravenously once on Day 1 of the administration cycle at a dose of:
(i) 600 mg to a patient weighing ≥10 to <20 kg;
(ii) 900 mg to a patient weighing ≥20 to <30 kg;
(iii) 1200 mg to a patient weighing ≥30 to <40 kg;
(iv) 2400 mg to a patient weighing ≥40 to <60 kg; or
(v) 2700 mg to a patient weighing ≥60 kg, and
(b) subcutaneously on Day 15 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

93. The kit of claim 91 or 92, wherein the therapeutic anti-C5 antibody, or antigen-binding fragment thereof, is provided in a 3 mL vial comprising Type I borosilicate glass for (a) intravenous administration and (b) subcutaneous administration to a patient weighing ≥10 to <20 kg.

94. A kit for treating PNH or aHUS in a pediatric human patient, wherein the patient is ravulizumab-experienced, comprising:
(a) a dose of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively; and
(b) instructions for using the therapeutic anti-C5 antibody, or antigen-binding fragment thereof, in the method of any one of claims 31-89.

95. The kit of claim 94, wherein the therapeutic anti-C5 antibody, or antigen-binding fragment thereof, is administered:
subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

96. The kit of any one of claims 91-95, wherein the pediatric human patient is less than 21 years of age.

97. The kit of claim 96, wherein the pediatric human patient is between 2 and 18 years of age.

98. The kit of any one of claims 91-97, wherein the therapeutic anti-C5 antibody, or antigen-binding fragment thereof, is administered to a site selected from the patient's arm, abdomen, and thigh.

99. A therapeutic anti-C5 antibody, or antigen-binding fragment thereof, for use in treatment of a pediatric human patient with PNH or aHUS, wherein the patient is complement inhibitor treatment-naïve or eculizumab-experienced, the treatment comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
(a) intravenously once on Day 1 of the administration cycle at a dose of:
(i) 600 mg to a patient weighing ≥10 to <20 kg;
(ii) 900 mg to a patient weighing ≥20 to <30 kg;
(iii) 1200 mg to a patient weighing ≥30 to <40 kg;
(iv) 2400 mg to a patient weighing ≥40 to <60 kg; or
(v) 2700 mg to a patient weighting ≥60 kg, and
(b) subcutaneously on Day 15 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

100. Use of a therapeutic anti-C5 antibody, or antigen-binding fragment thereof, in the manufacture of a medicament for treating aHUS or PNH in a pediatric human patient in need thereof, wherein the therapeutic anti-C5 antibody, or antigen-binding fragment thereof comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is complement inhibitor treatment-naïve or eculizumab-experienced, and wherein the therapeutic anti-C5 antibody, or antigen-binding fragment thereof, is formulated for administration by:
(a) intravenously once on Day 1 of the administration cycle at a dose of:
(i) 600 mg to a patient weighing ≥10 to <20 kg;
(ii) 900 mg to a patient weighing ≥20 to <30 kg;
(iii) 1200 mg to a patient weighing ≥30 to <40 kg;
(iv) 2400 mg to a patient weighing ≥40 to <60 kg; or
(v) 2700 mg to a patient weighting ≥60 kg, and
(b) subcutaneously on Day 15 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

101. A therapeutic anti-C5 antibody, or antigen-binding fragment thereof, for use in treatment of a pediatric human patient with PNH or aHUS, wherein the patient is ravulizumab-experienced, the treatment comprising administering to the patient during an administration cycle an effective amount of a therapeutic anti-C5 antibody, or antigen-binding fragment thereof, comprising CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the therapeutic anti-C5 antibody, or antigen-binding fragment thereof, is administered:
subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg.

102. Use of a therapeutic anti-C5 antibody, or antigen-binding fragment thereof, in the manufacture of a medicament for treating aHUS or PNH in a pediatric human patient in need thereof, wherein the therapeutic anti-C5 antibody, or antigen-binding fragment thereof, comprises CDR1, CDR2, and CDR3 heavy chain sequences as set forth in SEQ ID NOs: 19, 18, and 3, respectively, and CDR1, CDR2, and CDR3 light chain sequences as set forth in SEQ ID NOs: 4, 5, and 6, respectively, wherein the patient is ravulizumab-experienced, and wherein the therapeutic anti-C5 antibody, or antigen-binding fragment thereof, is formulated for administration:
subcutaneously on Day 1 of the administration cycle and every week thereafter at a dose of:
(i) 150 mg to a patient weighing ≥10 to <20 kg;
(ii) 245 mg to a patient weighing ≥20 to <40 kg; or
(iii) 490 mg to a patient weighing ≥40 kg once per week.

103. The therapeutic anti-C5 antibody, or antigen-binding fragment thereof, of claim 99 or 101, or the use of claim 100 or 102, wherein the pediatric human patient is less than 21 years of age.

104. The therapeutic anti-C5 antibody, or antigen-binding fragment thereof, of any one of claims 99, 101, and 103, or the use of any one of claims 100, 102, and 103, wherein the pediatric human patient is between 2 and 18 years of age.

105. The therapeutic anti-C5 antibody, or antigen-binding fragment thereof, of any one of claims 99, 101, and 103-104, or the use of any one of claims 100 and 102-104, configured for subcutaneous administration to a site selected from the patient's arm, abdomen, and thigh.


SEQUENCE LISTING (50 sequences)

SEQ ID NO: 1 - protein, 10 aa, synthetic construct
GYIFSNYWIQ

SEQ ID NO: 2 - protein, 17 aa, synthetic construct
EILPGSGSTEYTENFKD

SEQ ID NO: 3 - protein, 13 aa, synthetic construct
YFFGSSPNWYFDV

SEQ ID NO: 4 - protein, 11 aa, synthetic construct
GASENIYGALN

SEQ ID NO: 5 - protein, 7 aa, synthetic construct
GATNLAD

SEQ ID NO: 6 - protein, 9 aa, synthetic construct
QNVLNTPLT

SEQ ID NO: 7 - protein, 122 aa, synthetic construct [sequence omitted]

SEQ ID NO: 8 - protein, 107 aa, synthetic construct [sequence omitted]

SEQ ID NO: 9 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, synthetic construct [sequence omitted]

SEQ ID NO: 12 - protein, 122 aa, synthetic construct [sequence omitted]

SEQ ID NO: 13 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, synthetic construct
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, synthetic construct
GASENIYHALN

SEQ ID NO: 18 - protein, 17 aa, synthetic construct
EILPGSGHTEYTENFKD

SEQ ID NO: 19 - protein, 10 aa, synthetic construct
GHIFSNYWIQ

SEQ ID NO: 20 - protein, 448 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, synthetic construct
SYAIS

SEQ ID NO: 22 - protein, 17 aa, synthetic construct
GIGPFFGTANYAQKFQG

SEQ ID NO: 23 - protein, 7 aa, synthetic construct
DTPYFDY

SEQ ID NO: 24 - protein, 11 aa, synthetic construct
SGDSIPNYYVY

SEQ ID NO: 25 - protein, 7 aa, synthetic construct
DDSNRPS

SEQ ID NO: 26 - protein, 11 aa, synthetic construct
QSFDSSLNAEV

SEQ ID NO: 27 - protein, 116 aa, synthetic construct
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, synthetic construct
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, synthetic construct
NYIS

SEQ ID NO: 30 - protein, 17 aa, synthetic construct
IIDPDDSYTEYSPSFQG

SEQ ID NO: 31 - protein, 8 aa, synthetic construct
YEYGGFDI

SEQ ID NO: 32 - protein, 11 aa, synthetic construct
SGDNIGNSYVH

SEQ ID NO: 33 - protein, 7 aa, synthetic construct
KDNDRPS

SEQ ID NO: 34 - protein, 9 aa, synthetic construct
GTYDIESYV

SEQ ID NO: 35 - protein, 116 aa, synthetic construct
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, synthetic construct
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, synthetic construct
SSYYVA

SEQ ID NO: 38 - protein, 17 aa, synthetic construct
AIYTGSGATYKASWAKG

SEQ ID NO: 39 - protein, 13 aa, synthetic construct
DGGYDYPTHAMHY

SEQ ID NO: 40 - protein, 11 aa, synthetic construct
QASQNIGSSLA

SEQ ID NO: 41 - protein, 7 aa, synthetic construct
GASKTHS

SEQ ID NO: 42 - protein, 12 aa, synthetic construct
QSTKVGSSYGNH

SEQ ID NO: 43 - protein, 123 aa, synthetic construct
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, synthetic construct
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC